(19) United States
(12) Patent Application Publication
(10) Pub. No.: US 2013/0316989 A1
(43) Pub. Date: Nov. 28, 2013

(54) COMPOSITIONS AND METHODS FOR THE TREATMENT OF INFLAMMATORY DISEASE

(71) Applicant: RESOLVYX PHARMACEUTICALS, INC., Cambridge, MA (US)

(72) Inventor: Per Gjorstrup, Cambridge, MA (US)

(73) Assignee: RESOLVYX PHARMACEUTICALS, INC., Cambridge, MA (US)

(21) Appl. No.: 13/959,337

(22) Filed: Aug. 5, 2013

Related U.S. Application Data
(63) Continuation of application No. 13/120,493, filed on Apr. 11, 2011, now abandoned.
(60) Provisional application No. 61/194,066, filed on Sep. 23, 2008.

(51) Int. Cl. A61K 31/606; A61K 45/06; A61K 31/4439; A61K 31/20; A61K 31/336

(52) U.S. Cl. CPC A61K 31/606; A61K 31/20; A61K 31/336; A61K 31/4439; A61K 45/06
USPC 514/166; 514/560; 514/475; 514/342

(57) ABSTRACT

The invention relates to methods of treating inflammatory disease comprising administering a compound of formula A, a compound of any one of formulae 1-49 or I-III, a lipoxin compound, or an oxylipin compound.

### RELATED APPLICATIONS

This application claims the benefit of priority to U.S. Provisional Patent Application No. 61/194,066, Sep. 23, 2008, which application is hereby Incorporated by reference in its entirety.

### BACKGROUND

Nuclear receptors are a superfamily of regulatory proteins that are structurally and functionally related and whose ligands are, for example, steroids, retinoids, vitamin D and thyroid hormones (see, e.g., Evans (1988) Science 240: 889-895). These proteins bind to cis-acting elements in the promoters of their target genes and modulate gene expression in response to ligands for the receptors.

Nuclear receptors can be classified based on their DNA bind properties (see, e.g., Evans (1988) Science 240:889-895; and Glass (1994) Endocr. Rev. 15:391-407). For example, one class of nuclear receptors (including the glucocorticoid, estrogen, androgen, progestin and mineralocorticoid receptors bind as homodimers to hormone respond element (HREs) organized as inverted repeats. A second class of receptors (including those activated by retinoic acid, thyroid hormone, vitamin D₃, fatty acids/peroxisome profliferators and ecdysone) bind to HREs as heterodimers with a common partner, the retinoid X receptors (i.e., RXRs, also known as the 9-cis retinoic acid receptors; see, e.g., Levin et al. (1992) Nature 355:359-361, and Heyman et al. (1992) Cell 688:397-406).

Included, in the nuclear receptor superfamily of regulatory proteins are Endocrine Receptors, Adopted Orphan Receptors, and Orphan Receptors. The search for activators for orphan receptors has led to the discovery of previously unknown signaling pathways. Adopted Orphan Receptors are receptors for which endogenous ligands have been identified (such as low affinity dietary lipids). These Adopted Orphan Receptors have been identified as targets for therapeutic compounds.

Nuclear receptor activity has been implicated in a variety of diseases and disorders, including, but not limited to, hypercholesterolemia (see, e.g., International Patent Application Publication No. WO 00/57915), osteoporosis and vitamin deficiency (see, e.g., U.S. Pat. No. 6,316,5103), hyperlipoproteinemia (see, e.g., International Patent Application Publication No. WO 01/60818). hypertriglyceridemia, lipodystrophy, hyperglycemia and diabetes mellitus (see, e.g., International Patent Application Publication No. WO 01/82917), atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), acne (see, e.g., International Patent Application Publication No. WO 00/49992), and cancer, Parkinson's disease and Alzheimer's disease (see, e.g., International Patent Application Publication No. WO 00/17334). Activity of nuclear receptors, including LXRs, FXR and PPAR, and orphan nuclear receptors have been implicated in physiological processes including, but not limited to, bile acid biosynthesis, cholesterol metabolism or catabolism, and modulation of cholesterol 7α-hydroxylase gene (CYP7A1) transcription (see, e.g., Chiang et al. (2000) J. Biol. Chem. 275:10918-10924), HDL metabolism (see, e.g., Urizar et al. (2000) J. Biol. Chem. 275:39313-39317 and International Patent Application Publication No. WO 01/03705), and increased cholesterol efflux and increased expression of ATP binding Cassette transporter protein (ABC1) (see, e.g., International Patent Application Publication No. WO 00/78972).

The peroxisome proliferator activated receptors (PPARs) are members of the nuclear receptor gene family that are activated by fatty acids and fatty acids metabolites. The PPARs belong to the subset of nuclear receptors that function as heterodimers with the 9-cis retinoic acid receptor (RXR). Three subtypes, designated PPARα, PPARγ and PPARβ/δ, are found in species ranging from Xenopus to humans. The expression profile of each isoform differs significantly from the others. While PPARα is expressed primarily, but not exclusively in liver. PPARγ is expressed primarily in adipose tissue, and PPARβ/δ is expressed obiquitously. Studies of the individual PPAR isoforms and ligands have elucidated their regulation of processes involved in insulin resistance and diabetes, as well as lipid disorders, such as hyperlipidemia and dyslipidemia. PPARβ/δ agonists are believed to mediated anti-inflammatory effects. Indeed, treatment of LPS-stimulated macrophages with a PPARβ/δ agonist has been observed to reduce the expression of iNOS IL12, and IL-6 (Welch, J. S., et al.; Proc Natl Acad Sci 100:6712-67172003). PPARα and PPARγ receptors have been implicated in diabetes mellitus, cardiovascular disease, obesity, and gastrointestinal disease, such as inflammatory bowel disease and other inflammation related illnesses. Such inflammation related illnesses include, but are not limited to Alzheimer's disease, Crohn's disease, rheumatoid arthritis, psoriasis, and ischemia reperfusion injury.

The PPARγ agonists, including glitazones, also known as thiazolidinediones (e.g., 5-benzylthiazolidine-2,4-diones) and non-thiazolidinediones (e.g., glitizars), a class of compounds with potential for ameliorating many symptoms of Type 2 diabetes, operated by substantially increasing insulin sensitivity in muscle, liver and adipose tissue. This results in partial or complete correction of the elevated plasma levels of glucose without occurrence of hypoglycemia. The currently marketed glitazones are agonists of the peroxisome proliferator activated receptor (PPAR) gamma subtype, PPARγ agonism is generally believed to be responsible for the improved insulin sensitization that is observed with the glitazones. Although thiazolidinediones have been shown to increases insulin sensitivity by binding to PPARγ receptors, this treatment also produces unwanted side effects such as weight gain, edema, and, for troglitazone, liver toxicity. New PPAR agonists that are being developed for treatment of Type 2 diabetes and/or dyslipidemia are agonists of one or more of the PPAR alpha, gamma and delta subtypes.

Since their initial discovery, the thiazolidinediones have also been found to have anti-inflammatory properties documented in both acute and chronic experimental conditions such as prevention of iscehmia/reperfusion damage. TNBS-induced colitis, collagen-induced arthritis and other models. (See, for example, Ye, Y. et al. Am. J. Physiol. Heart Circ. Physiol. (2006) 291:H1158-H1169; Sanchez-Hidalgo, M. et al. Biochem. Pharmacol. (2005) 69:1733-1744; and Moraes, L. A. et al. Pharmacol. Therapeut. (2006) 110:371-385).

As Orphan Nuclear Receptors continue to be deorphanized or synthetic ligands acting selectively on a specific orphan receptor are identified, the role of these receptors in the homeostatic control of inflammation and as targets for novel drugs to treat inflammatory diseases is becoming increasingly important. Recently, the Liver X Receptor (LXR) group of receptors was demonstrated to have a role in the Integrated control of inflammation and metabolism. (See, for example, Zelcer, N. et al. J, Clin. Investig. (2006) 116:607-614.)

LXRα is found predominantly in liver, with lower levels found in kidney, intestine, spleen and adrenal tissue (see, e.g., Willy, et al. (1995) Gene Dev. 9(9);1033-1045). LXRβ is abiquitous in mammal and was found in nearly all tissues examined. LXRs are activated by certain naturally occurring, oxidized derivatives of cholesterol (see, e.g., Lehmann, et al. (1997) J. Biol. Chem. 272(6):3137-3140). LXRα is activated by oxycholesterol and promotes cholesterol metabolism (Peet et al. (1998) Cell 93:693-704). Thus, LXRs appear to play a role in e.g., cholesterol metabolism (see, e.g., Janowski, et al. (1996) Nature 383:728-731).

### SUMMARY OF INVENTION

The present invention provides methods for the treatment of inflammatory disease in a patient comprising conjointly administering to the patient; a) a compound of formula A, a compound of nay one of formulae 1-49 or I-III, a lipoxin compound, an oxylipin compound, or a combination of aspirin and an omega-3 fatty acids; with b) a PPAR agonist (e.g., a PPARα, PPARβ/δ, or a PPARγ agonist), an LXR agonist (e.g., an LXRα or LXRβ agonist), and RXR agonist (e.g., an RXRα, RXRβ, or an RXRγ agonist), an HNF-4 agonist, or a sirtiun-activating compound.

The present invention further provides methods for the treatment of a complex disorder having an inflammatory component in a patient comprising conjointly administering to the patent; a) a compound of formula A, a compound of any one of formulae 1-49 or I-III, a lipoxin compounds, an oxylipin compound, or a combination of aspirin and an omega-3 fatty acid; with b) a PPAR agonist (e.g., a PPARα, PPAR β/δ, or a PPARγ agonist), an LXR agonist (e.g., an LXRα or LXRβ agonist), an RXR agonist (e.g., an RXRα, RXRβ, or an RXRγ agonist), and HNF-4 agonist, or a sirtuin-activating compound.

The present invention also provides pharmaceutical compositions comprising: a) a compound of formula A, a compound of any one of formulae 1-49 or I-III, a lipoxin compound, an oxylipin compound, or a combination of aspiring and an omega-3 fatty acids; and b) a PPAR agonist (e.g., a PPARα, PPARβ/δ, or a PPARγ agonist), and LXR agonist (e.g., an LXRα or LXRβ agonist), an RXR agonist (e.g., an RXRα, RXRβ, or an RXRγ agonist), an HNF-4 agonist, or a sirtuin-activating compound.

The present invention further provides methods for the treatment of type 1 diabetes in a patient comprising administering to the patient a compound of formula A, compound of any one of formulae 1-49 or I-III, a lipoxin compound, an oxylipin compound, or a combination of aspirin and an omega-3 fatty acid.

### DETAILED DESCRIPTION OF THE INVENTION

The present invention provides a method of treating inflammatory disease in a patient comprising conjointly administering to said patient: a) a PPAR agonist (e.g., a PPARα, PPAR β/δ, or a PPARγ agonist), an LXR agonist (e.g., an LXRα or LXRβ agonist), an RXR agonist (e.g., an RXRα, RXRβ, or an RXRγ agonist), and HNF-4 agonist, with b) a compound of formula A, a compound of any one of formulae 1-49 or I-III, a lipoxin compound, an oxylipin compound, or a combination of aspiring and an omega-3 fatty acid.

The present invention also provides a method of treating inflammatory disease in a patient comprising conjointly administering to said patient a sirtuin-activating compound with a compound of formula A, a compound of any one of formulae 1-49 or I-III, a lipoxin in compound, an oxylipin compound, or a combination of aspiring and an omega-3 fatty acid.

Compounds of formula A, compounds of any one of formulae 1-49 or I-III, lipoxins, and oxylipins are capable of resolving inflammation. The combination of aspirin and an omega 3-fatty acid produces active metabolites that are also capable of resolving inflammation. PPAR agonists, such as the triazolidinediones, although originally developed as insulin sensitizers for the treatment of non-insulin-dependent diabetes type 2, have been found to have anti-inflammatory properties as well. The full therapeutic potential of PPAR agonists as a monotherapy is diminished due to treatment-limiting adverse events. For example, the administration of PPAR agonists, such as thiazolidinediones, can result in side effects including weight gain, edema, fluid retention that may aggravate heart failure, and, in some cases, liver toxicity. Advantageously, treatment of inflammatory disease with a combination of: a) a PPAR or LXR agonist; and b) a compound of formula A, compound of any one of formulae 1-49 or I-III, lipoxin, oxylipin, or a combination of aspirin and a omega-3 fatty acid enhance the anti-inflammatory properties of both classes of compounds while reducing the effects associated with high doses of PPAR agonists alone.

Examples of inflammatory diseases that may be treated or prevented by the conjoint administration of a PPAR agonist (e.g., a PPARα, PPAR β/δ, or a PPARγ agonist), an LXR agonist (e.g., an LXRα or LXRβ agonist), an RXR agonist (e.g., an RXRα, RXRβ, or an RXRγ agonist), an HNF-4 agonist, or a sirtuin-activating compound and a compound of formula A, a compound of any one of formulae 1-49 or I-III, a lipoxin compound, an oxylipin compound, or a combination of aspirin and an omega-3 fatty acid, include inflammation of the lungs, joints, connective tissue, eyes, nose, bowel, kidney, liver, skin, central nervous system, vascular system, heart, or adipose tissue. In certain embodiments, inflammatory diseases which may be treated by the current invention include inflammation due to the infiltration of leukocytes or other immune effector cells into affected tissue. Other relevant examples of inflammatory diseases which may be treated by the present invention include inflammation caused by infectious agents, including but not limited to viruses, bacteria, fungi, and parasites.

Inflammatory lung conditions include asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Inflammatory eye conditions include uveitis (including iritis), conjunctivitis, scleritis, and keratoconjunctivitis sicca. Inflammatory bowel conditions include Crohn's disease, ulcerative colitis and distal proctitis.

Inflammatory skin diseases include conditions associated with cell proliferation, such as psoriasis, eczema, and dermatitis (e.g., eczematous dermatitides, topic and seborrheic dermatitis, allergic or irritant contact dermatitis, eczema craquelee, photoallergic dermatitis, photoxicdermatitis, phytophotodermatitis, radiation dermatitis, and stasis dermatitis). Other inflammatory skin diseases include, but are not limited to, ulcers and erosions resulting from trauma, burns, bullous disorders, or ischemia of the skin or mucous membranes, several forms of ichthyoses, epidermolysis bullosae, hypertrophic scars, keloids, cutaneous changes of intrinsic aging, photo aging, frictional blistering caused by mechanical shearing of the skin and cutaneous atrophy resulting from the topical use of corticosteroids. Additional inflammatory skin conditions include inflammation of mucous membranes, such as cheilitis, nasal irritation, mucositis and vulvovaginitis.

Inflammatory disorders of the endocrine system include, but are not limited to, autoimmune thyroiditis (Hashimoto's disease), Type I diabetes, inflammation in liver and adipose tissue associated with Type II diabetes, and acute and chronic inflammation of the adrenal cortex. Inflammatory diseases of the cardiovascular system includes, but are not limited to, coronary infarct damage, peripheral vascular disease, myocarditis, vasculitis, revascularization of stenosis, atherosclerosis, and vascular disease associated with Type II diabetes.

Inflammatory condition of the kidney include, but are not limited to, glomerulonephritis, interstitial nephritis, lupus nephritis, nephritis secondary to Wegener's disease, acute renal failure secondary to acute nephritis, post-obstructive syndrome and tubular ischemia.

Inflammatory diseases of the liver include, but are not limited to, hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis. Inflammatory diseases of the adipose tissues include, but are not limited to, obesity.

Inflammatory diseases of the central nervous system include, but are not limited to, multiple sclerosis and neurodegenerative diseases such as Alzheimer's disease, Parkinson'disease or dementia associated with HIV infection. Other inflammatory diseases include periodontal disease, tissue necrosis in chronic inflammation, endotoxin shock, smooth muscle proliferation disorders, tissue damage following ischemia reperfusion injury, and tissue rejection following transplant surgery.

It should be noted that the inflammatory diseases cited above are meant to be exemplary rather than exhaustive. Those skilled in the art would recognize that additional inflammatory diseases (e.g., systemic or local immune imbalance or dysfunction due to an injury, infection, insult, inherited disorder, or an environmental intoxicant or perturbant to the subject's physiology) may be treated by the methods of the current invention. Thus, the methods of the current invention may be used to treat or prevent any disease which has an inflammatory component, including, but not limited to, those diseases cited above.

The present invention also provides methods for treating or preventing arthritis, inflammatory bowel disease, uveitis, ocular inflammation, asthma, pulmonary inflammation, cystic fibrosis, psoriasis, arterial inflammation, cardiovascular diseases, multiple sclerosis, or neurodegenerative disease by conjointly administering an effective amount of: a) a PPAR agonist (e.g., a PPARα, PPAR β/δ, or a PPARγ agonist), an LXR agonist (e.g., an LXRα or LXRβ agonist), an RXR agonist (e.g., an RXRα, RXRβ, or an RXRγ agonist), and HNF-4 agonist, or a sirtuin-activating compound; with b) a compound of formula A, a compound of any one of formulae 1-49 or I-III, a lipoxin compound, an oxylipin compounds, or a combination of aspirin and an omega-3 fatty acid.

The present invention also provides a method of treating complex disorders having an inflammatory component in a patient comprising administering to said patient a compound of formula A, a compound of any one of formulae 1-49 or I-III, a lipoxin compound, an oxylipin compound, or a combination of aspirin and an omega-3 fatty acid. In certain embodiments, the complex disorder having an inflammatory component is type 2 diabetes or obesity.

The present invention further provides a method of treating complex disorders having an inflammatory component in a patient, comprising conjointly administering to said patient: a) a PPAR agonist (e.g., a PPARα, PPAR β/δ, or a PPARγ agonist), an LXR agonist (e.g., an LXRα or LXRβ agonist), an RXR agonist (e.g., an RXRα, RXRβ, or an RXRγ agonist), and HNF-4 agonist, or a sirtuin-activating compound; with b) a compound of formula A, a compound of any one of formulae 1-49 or I-III, a lipoxin compound, an oxylipin compounds, or a combination of aspirin and an omega-3 fatty acid. In certain embodiments, the complex disorder having an inflammatory component is type 2 diabetes or obesity. Thiazolidinediones, a class of PPAR agonists, is known for the treatment of type 2 diabetes. As such, a treatment particularly well-suited for a complex disorder having an inflammatory component, such as type 2 diabetes, is the conjoint administration of a) a PPAR agonist (e.g., a PPARα, PPAR β/δ, or a PPARγ agonist); and b) a compound of formula A, a compound of any one of formulae 1-49 or I-III, a lipoxin compound, an oxylipin compounds, or a combination of aspirin and an omega-3 fatty acid.

The present invention further provides a method of treating or preventing a neurological condition in a patient comprising conjointly administering to said patient: a) a PPAR agonist (e.g., a PPARα, PPAR β/δ, or a PPARγ agonist), an LXR agonist (e.g., an LXRα or LXRβ agonist), an RXR agonist (e.g., an RXRα, RXRβ, or an RXRγ agonist), an HNF-4 agonist, or a sirtuin-activating compound; with b) a compound of formula A, a compound of any one of formulae 1-49 or I-III, a lipoxin compound, an oxylipin compound, or a combination of aspirin and a omega-3 fatty acid. In certain embodiments, the neurological condition may be selected from neurodegeneration of dementia associated with HIV infection, Alzheimer's disease, addiction, alcohol-related disorders, decision analysis, degenerative, neurological disorders, dementia, neurological disorders, neuromuscular disorders, psychiatric disorders, brain injury, trauma, neuronal inflammation, or multiple sclerosis.

In methods of the invention wherein a PPAR agonist (e.g., a PPARα, PPAR β/δ, or a PPARγ agonist) is administered conjointly with a compound of formula A, a compound of any one of formulae 1-49 or I-III, a lipoxin compound, an oxylipin compound, or a combination of aspirin and a omega-3 fatty acid, the PPAR agonist may be any suitable PPAR agonist. PPAR agonists suitable for said conjoint administration include, but are not limited to, GW409544, LY-518674, LY-510929, TZD18, LTB4, oleylethanolamide, LY-465608, pirinixic acid, fatty acids (e.g., docohexaenoic acid, arachidonic acid, linoleic acid, C-6-C18 fatty acid, and eicosatetraynoic acid), ragaglitazar, AD-5061, fenofibric acid, GW7647, GW9578, TAK-559, KRP-297/MK-0767, eicosatetraenoic acid, farglitazar, reglitazar, DRF 2519, pristanic acid, bezafibrate, clofibrate, 8S-hydroxyeicosatetraenoic acid, GW2331, NS-220, pterostilbene, tetradecylglycidic acid, ortylthiopropionic acid, WY14643, ciprofibrate, gemfibrozil, muraglitazar, tesaglitazar, eicosanoids (e.g., 15d-PGJ₂, PGJ₂, protacyclin, PGI_{W}, PGA_{1/2}, PGB₂, 8-hydroxyeicosapentaienoic acid, 8(R)hydrxyeicosatetraenoic acid, 8-(S)hydroxyeicosatetraenoic acid, 12-hydroxyeicosatetraenoic acid LTB₄, 9-(R/S)hydroxyeicosatetraenoic acid, 13-(R/S)hydroxyeicosatetraenoic acid, 20,8,9-hydroxyeicosatetraenoic acid, 20,11,12-hydroxyepoxyeicosatrienoic acid, and 20,14,15-hydroxyepoxyeicosatrienoic acid), GW0742X, GW2433, GW9578, GW0742, L-783483, GW501516, retinoic acid, L-796449, L-165461, L-165041, SB-219994, LY-510929, AD-5061, L-764406, GW00072, nTzDpa, troglitazone, LY-465608, pioglitazone, SB-219993, 5-aminosalicyclic acid, GW1929, L-796449, GW7845, 2-cyano-3,12-dioxooleana-1,9-dien-28-oic acid, L-783483, L-165461, AD5075, flnorenylmethoxycarbonyl-L-leucine, CS-045, indomethacin, rosiglitazone (BRL49653), SB-236636, GW2331, PAT5A, MCC555, bisphenol A diglycidyl ether, GW409544, GW9578, TAK-559, reglitazar, GW9578 ciglitazone, DRF2519, LG10074, ibuprofen, diclofenac, fenofibrate, naviglitazar, or pharmaceutically acceptable salts thereof.

In methods of the invention, wherein an LXR agonist (e.g., an LXRα or LXRβ agonist) is administered conjointly with a compound of formula A, a compound of any one of formulae 1-49 or I-III, a lipoxin compound, an oxylipin compound, or a combination of aspirin and a omega-3 fatty acid, the LXR agonist may be chosen from any suitable LXR agonist. LXR agonists suitable for said conjoint administration include, but are not limited to TO901317, GW3965, T1317, acetyl-podocarpic dimer (APD), or pharmaceutically acceptable salts thereof. Other examples of LXR agonists suitable for said conjoint administration may be found in US Patent Application No. 2006/205819 and references cited therein.

In methods of the invention, wherein an HNF-4 agonist is administered conjointly with a compound of formula A, a compound of any one of formulae 1-49 or I-III, a lipoxin compound, an oxylipin compound, or a combination of aspirin and an omega-3 fatty acid, the HNF-4 agonist may be chosen from any suitable HNF-4 agonist.

In methods of the invention, wherein an RXR agonist (e.g., an RXRα, RXRβ, or RXRγ agonist) is administered conjointly with a compound of formula A, a compound of any one of formulae 1-49 or I-III, a lipoxin compound, an oxylipin compound, or a combination of aspirin and an omega-3 fatty acid, the RXR agonist may be chosen from any suitable RXR agonist. RXR agonists suitable for said conjoint administration include, but are not limited to LG 100268 (i.e. 2-[1-3,5,5,8,8-pentamethyl-5,6,7,8-tetrahydro-2-naphthyl)-cyclopropyl]-pyridine-5-carboxylic acid), LGD 1069 (i.e. 4-[(3,5,5,8,8-pentamethyl-5,6,7,8-tetrahydro-2-naphthyl)-2-carbonyl]-benzoic acid), AGN 194204, 9-cis-retinoic acid, AGN 191701, bexarotene, BMS 649, and analogs, derivatives and pharmaceutically acceptable salts thereof. The structures and syntheses of LG 100268 and LGD 1069 are disclosed in Boehm, et al. J. Med. Chem. 38(16):3146-3155, 1994, incorporated by reference herein.

In methods of the invention wherein a sirtuin-activating compound is administered conjointly with a compound of formula A, a compound of any one of formulae 1-49 or I-III, a lipoxin compound, an oxylipin compound, or a combination of aspirin and an omega-3 fatty acid, the sirtuin-activating compound may be any suitable sirtuin-activating compound. Sirtuin-activating compounds suitable for said conjoint administration include, but are not limited to, those sirtuin-activating compounds described in the following applications: WO2007019416, WO2007008548, WO2006105440, WO2006127987, WO2006105403, WO2006094237, WO2006094236, WO2006094235, WO2006076681, WO2006079021, US2007043050, US2007037809, US2007037827, US2006276393, WO2006094258, WO2006078941, WO2005069998, WO2006096780, WO2007104867, US2007212395, WO2006138418, US2006292099, JP2006298876, and US2006025337, all of which are herein incorporated by reference,

The present invention further provides a method of treating type 1 diabetes in a patient comprising administering to said patient a compound of formula A, a compound of any one of formulae 1-49 or I-III, a lipoxin compound, an oxylipin compound, or a combination of aspirin and an omega-3 fatty acid. In certain embodiments, the present invention provides a method of treating a patient at risk of developing type 1 diabetes comprising administering to said patient a compound of formula A, a compound of any one of formulae 1-49 or I-III, a lipoxin compound of oxylipin compound, or a combination of aspirin and an omega-3 fatty acid. In certain embodiments, the present invention provides a method of treating a patient exhibiting warning signs of type 1 diabetes, such as extreme thirst; frequent urination; drowsiness or lethargy; sugar in urine; sudden vision changes; increased appetite; sudden weight loss; fruity, sweet, or wine-like odor on breath; heavy, labored breathing; stupor; and unconsciousness, comprising administering to said patient a compound of formula A, a compound of any one of formulae 1-49 or I-III a lipoxin compound, an oxylipin compound, or a combination of aspirin and an omega-3 fatty acid.

The present invention further provides a method for protecting, e.g., promoting the growth and/or survival of, beta cells of Islets of Langerhans from lipid- or glucose-triggered toxicity in a patient comprising administering to the patient a compound of formula A, a compound of any one of formulae 1-49 or I-III, a lipoxin compound, an oxylipin compound, or a combination of aspirin and a an omega-3 fatty acid.

Compounds suitable for use in methods of the invention include those of Formula A.

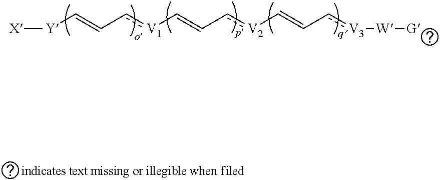

wherein:

each of W′ and Y′ is a bond or a linker independently selected from a ring containing up to 20 atoms or a chain of up to 20 atoms, provided that W′ and Y′ can independently include one or more nitrogen, oxygen, sulfur or phosphorous atoms, further provided that W′ and Y′ can independently include one or more substituents independently selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryly, or sulfonyl, further provided that W′ and Y′ can independently contain one or more fused carbocyclic, heterocyclic, aryl or heteroaryl rings, and further provided that when o′ is 0, and V₁ is

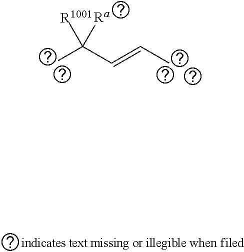

Y′ is connected to V₁ via a carbon atom;

V₁ is selected from

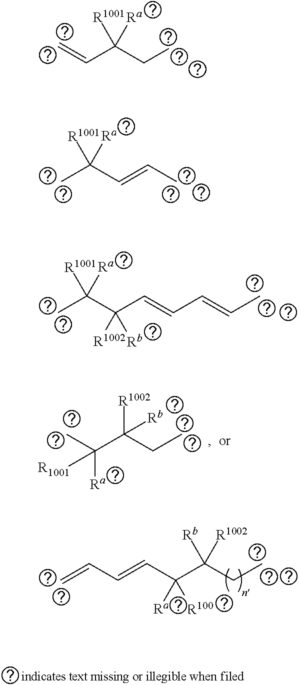

wherein when q′ is 0 and V₃ is a bond, n′ is 0 or 1; otherwise n′ is 1;

V₂ is selected from a bond,

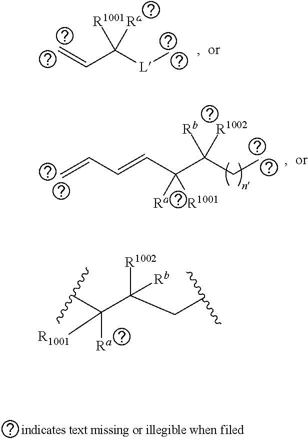

wherein:

L′ is selected from —C(R¹⁰⁰³)(R¹⁰⁰⁴)—, wherein each of R¹⁰⁰³ and R¹⁰⁰⁴ is independently selected from hydrogen, alkyl, alkenyl, alkynyl, perfluoroalkyl, alkoxy, aryl or heteroaryl, or R¹⁰⁰³ and R¹⁰⁰⁴ are connected together to form a carbocyclic or heterocyclic ring; when V₃ is

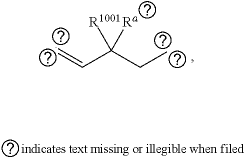

L′ is additionally selected from W′; and n′ is 0 or 1; V₃ is selected from a bond or

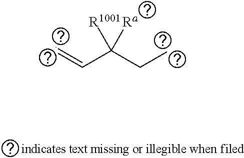

wherein:

each R¹⁰⁰¹ and R¹⁰⁰² is independently for each occurrence selected from hydrogen, alkyl, alkenyl, alkynyl, aryl, heteroaryl, alkylaryl, alkoxy, or halo, wherein a said alky- or aryl-containing moiety is optionally substituted with up to 3 independently selected substituents;

each of R^{a′} and R^{b′} is independently for each occurrence selected from —OR′ or —N(R′)₂, or adjacent R^{a′} and R^{b′} are taken together to form an epoxide ring having a cis or trans configuration, wherein each R′ is independently selected from hydrogen, alkyl, alkenyl alkynyl, aryl, heteroaryl, acyl, silyl, alkoxyacyl, aminoacyl, aminocarbonyl, alkoxycarbonyl, or a protecting group; or when V₁ is

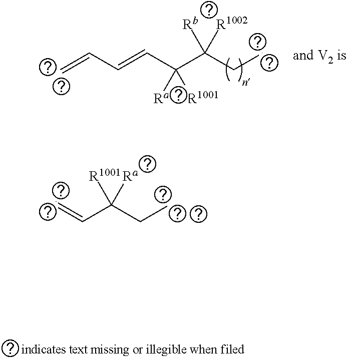

R¹⁰⁰² and R^{b′} are both hydrogen;

X′ is selected from —CN, —C(NH)N(R″)(R″), —C(S)—A′, —C(S)R″, —C(O)—A′, —C(O)—R″, —C(O)—SR″; —CO(O)—NH—S(O)₂—R″, —S(O)₂—A′, —S(O)₂—R″, S(O)₂N(R″)(R″), —P(O)₂—A′, —PO(OR″)—A′, -tetrazole, alkyltetrazole, or —CH₂OH, wherein
A′ is selected from —OR″, —N(R″)(R″) or —OM′;
each R″ is independently selected from hydrogen, alkyl, aryl, arylalkyl, heteroaryl, heteroarylalkyl or a detectable label molecule, wherein any alkyl- , aryl- or heteroaryl-containing moiety is optionally substituted with up to 3 independently selected substituents; and
M′ is a cation;

G′ is selected from hydrogen, halo, hydroxy, alkyl, aryl, arylalkyl, heteroaryl, heteroarylalkyl, alkoxy, aryloxy, carboxy, amino, alkylamino, dialkylamino, acylamino, carboxamido or a detectable label molecule, wherein any alky- , aryl- or heteroaryl-containing moiety is optionally substituted with up to 3 independently selected substituents:

o′ is 0, 1, 2, 3, 4 or 5;

p′ is 0, 1, 2, 3, 4 or 5;

q′ is 0, 1, or 2; and

o′+p′+q′ is 1, 2, 3, 4, 5 or 6;

wherein:

if V₂ is a bond, then q′ is 0, and V₃ is a bond;

if V₃ is

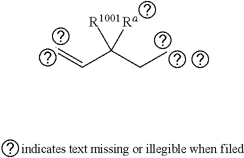

then o′ is 0, V₁ is

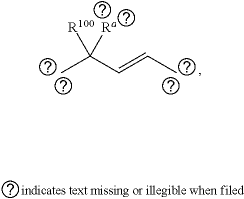

p′ is 1 and V₂ is

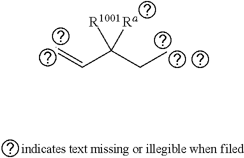

any acyclic double bond may be in a cis or a trans configuration or is optionally replaced by a triple bond; and

either one

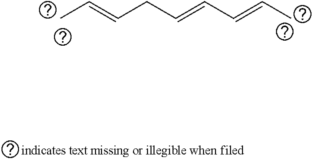

portion of the compound, if present, is optionally replaced by

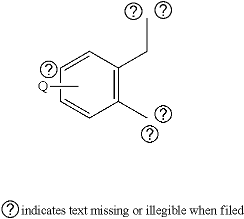

or one

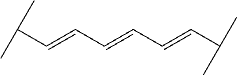

portion of the compound, if present, is optionally replaced by

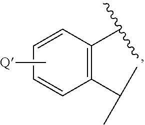

wherein Q′ represents one or more substituents and each Q′ is independently selected from halo, alkyl, alkenyl, alkynyl, cycloalkyl, aryl, heteroaryl, alkoxy, aryloxy, alkylcarbonyl, arylcarbonyl, alkoxycarbonyl, aryloxycarbonyl, amino, hydroxy, cyano, carboxy, alkoxycarbonyloxy, aryloxycarbonyloxy or aminocarbonyl.

In certain embodiments, V₁ is selected from

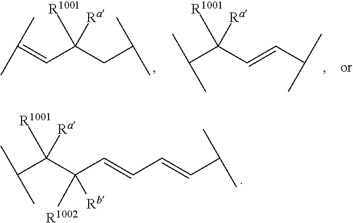

In certain embodiments, V₂ is selected from a bond,

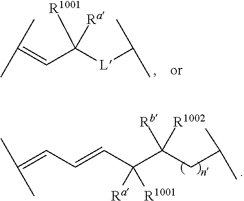

In certain embodiments, when q′ is 0 and V₃ is a bond, n′ is 0 or 1; otherwise n′ is 1.

In certain embodiments, p′ is 0, 1, 2, 3, or 5.

In certain embodiments, q′ is 0 or 1.

In certain embodiments, if V₁ is

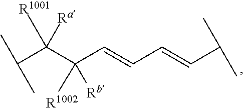

then o′ is 0 or 1, p′ is 1 or 2, o′+p′ is 1 or 2, V₂ is

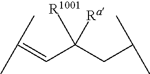

and V₃ is a bond.

In certain embodiments, if V₁ is

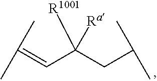

Then o′ is 3, 4 or 5, p′ is 0, 1 or 2, o′+p′ is 4 or 5, and V₂ is a bond.

In certain embodiments, if V₂ is a bond, then o′ is 0, 3, 4 or 5; p′ is 0, 1, 2 or 5, o′+p′ is 4 or 5, q′ is 0, and V₃ is a bond.

In certain embodiments, each of W′ and Y′ is independently selected from a bond or lower alkyl or heteroalkyl optionally substituted with one or more substituents independently selected from alkenyl, alkynyl, aryl, chloro, iodo, bromo, fluoro, hydroxy, amino, or oxo.

Compounds suitable for use in methods of the invention include those of Formula 1,

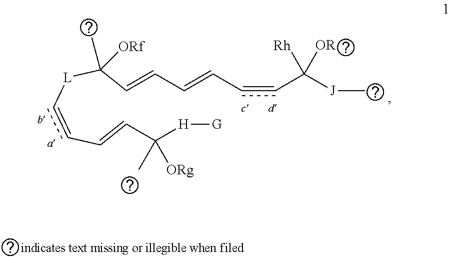

wherein
Carbons a′ and b′ are connected by a double bond or a triple bond;
Carbons c′ and d′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
Rh, Ri and Rj are independently selected from hydrogen, alkyl, alkenyl, alkynyl, perfluoroalkyl, aryl or heteroaryl;
I is selected from —CO(O)—E, —SO₂—E, —PO(OR)—E, where E is hydroxy, alkoxy, aryloxy, amino, alkylamino, dialkylamino, or arylamino; and R is hydrogen or alkyl;
J, L and H are linkers independently selected from a ring containing up to 20 atoms or a chain of up to 20 atoms, provided that J, L and H can independently include one or more nitrogen, oxygen, sulfur or phosphorous atoms, and further provided that J, L and H can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J, L and H can also contain one or more fused carbocyclic, heterocyclic, aryl or heteroaryl rings, and provided that linker J is connected to the adjacent C(R)OR group via a carbon atom;
G is selected from hydrogen, alkyl, perfluoroalkyl, alkenyl, alkynyl, aryl, heteroaryl, chloro, iodo, bromo, fluoro, hydroxy, alkoxy, aryloxy, carboxy, amino, alkylamino, dialkylamino, acylamino, or carboxamido;
   or pharmaceutically acceptable salts thereof.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

In certain embodiments, a compound for formula 1 is represented by formula 2.

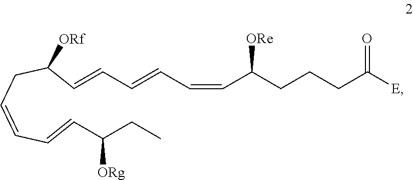

wherein

E, Re, Rf, and Rg are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 2 include:

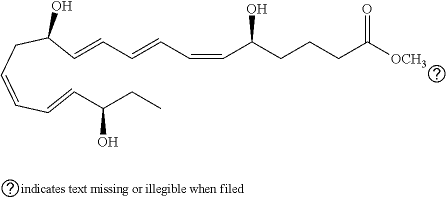 [SMILES: CC[C@@H](O)/C=C/C=C\C[C@@H](O)/C=C/C=C/C=C\[C@@H](O)CCCC(=O)OC]

In certain embodiments, a compound of formula 1 is represented by formula 3,

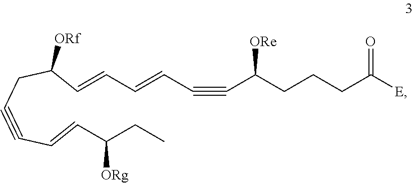

wherein

E, Re, Rf, and Rg are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 3 include:

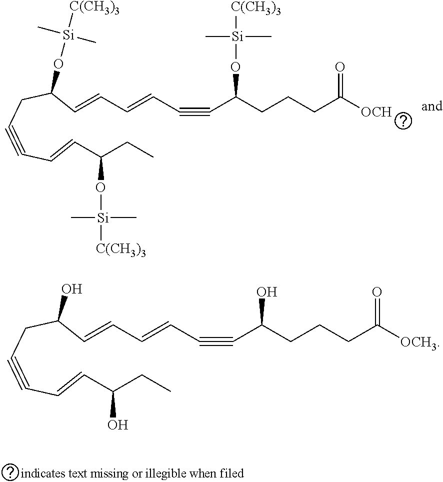

Further exemplary compounds of formula 1 include Compound X.

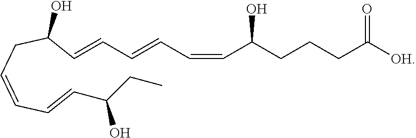 [SMILES: CC[C@@H](O)/C=C/C=C\C[C@@H](O)/C=C/C=C/C=C\[C@@H](O)CCCC(=O)O]

Other compounds suitable for use in methods of the invention include those of Formula 4,

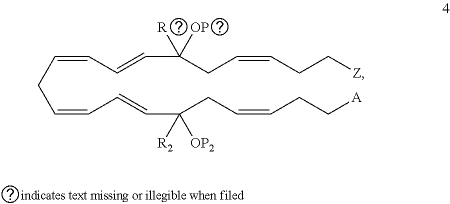

wherein

A is H or —OP₄;

P₁, P₂ and P₄ each individually is a protecting group or hydrogen atom;

R₁ and R₂ each individually is a substituted or unsubstituted, branched or unbranched alky, alkenyl, or alkynyl group, substituted or unsubstituted aryl group, substituted or unsubstituted, branched or unbranched alkylaryl group, halogen atom, hydrogen atom;

Z is —C(O)OR^{d}, —C(O)NR^{c}R^{e}, —C(O)H, —C(NH)NR^{e}R^{c}, —(S)H, —C(S)OR^{d}, —C(S)NR^{c}R^{c}, —CN, preferably a carboxylic acid, ester, amide, thioester, thiocarboxamide or a nitrile;

each R^{a}, if present, is independently selected from hydrogen, (C1-C6) alkyl, (C2-C6) alkenyl, (C2-C6) alkynyl, (C3-C8) cyclohexyl, (C4-C11) cycloalkylalkyl, (C5-C10) aryl, phenyl, (C6-C16) arylalkyl, benzyl, 2-6 membered heteroalkyl, 3-8 membered heterocyclyl, morpholinyl, piperazinyl, homopiperazinyl, piperidinyl, 4-11 membered heterocyclylalkyl, 5-10 membered heteroaryl and 6-16 membered heteroarylalkyl;

each R^{b}, if present, is a suitable group independently selected from ═O, —OR^{d}, (C1-C3) haloalkyloxy, —OCR₃, ═S, —SR^{d}═NR^{d}, ═NOR^{d}, —NR^{c}R^{c}, halogen, —CF₃, —CN, —NC, —OCN, —OCN, —SCN, —NO, —NO₂, ═N₂, —N₃—S(OR)R^{d}, —S(O)₂R^{d}, —S(O)₂OR^{d}, —S(O)NR^{e}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{ω})NR^{c}R^{c}, —[NHC(O)]ₐR^{d}, —[NR^{a}C(O)]ₐR^{d}, —[NHC(O)]ₐOR^{d}, —[NR^{a}C(O)]ₐOR^{d}, [NHC(O)]ₐNR^{c}R^{c}, —[NR^{a}C(O)]NR^{c}R^{c}, —[NHC(NH)]ₙNR^{c}R^{c} and —[NR^{a}C(NR^{a})]ₙNR^{d}R^{c};

each R^{c}, if present, is independently a protecting group or R^{a}, or, alternatively, two R^{c} taken together with the nitrogen atom to they are bonded form a 5 to 8-membered heterocyclyl or heteroaryl which optionally including one or more additional heteroatoms and optionally substituted with one or more of the same or different R^{a} or suitable R^{b} groups:

each n independently is an integer from 0 to 3;

each R^{d} independently is a protecting group or R^{a}; or pharmaceutically acceptable salts thereof.

Other compounds suitable for use in methods of the invention included those of Formula 5.

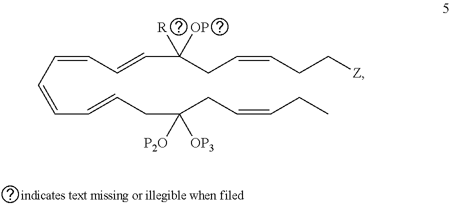

or pharmaceutically acceptable salts thereof, wherein
P₃ is a protecting group or hydrogen atom; and

P₃, P₂, R₁ and Z are as defined above in formula 4.

Exemplary compounds of formula 5 include compound 5a,

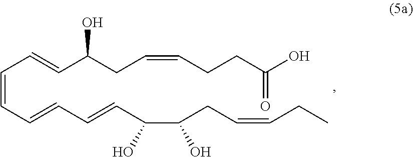 [SMILES: CC/C=C\C[C@H](O)[C@H](O)/C=C/C=C/C=C\C=C\[C@@H](O)C/C=C\CCC(=O)O]

and pharmaceutically acceptable salts and esters thereof.

Other compounds suitable for use in methods of the invention include those of Formula 6.

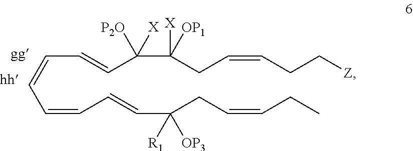

or pharmaceutically acceptable salts thereof, wherein the stereochemistry of the carbon gg′ to carbon hh′ bond is cis or trans;
each X represents hydrogen or taken together both X groups represent one substituted or unsubstituted methylene, and oxygen atom, a substituted or unsubstituted N atom, or a sulfur atom such that a three-membered ring is formed; and
P₁, P₂, P₃, R₁ and Z are as defined above.

Exemplary compounds of formula 6 include compound 6a.

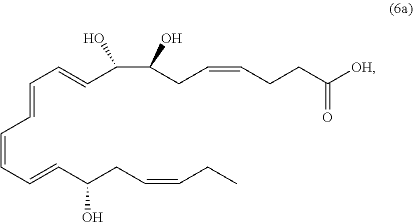 [SMILES: CC/C=C\C[C@H](O)/C=C/C=C\C=C\C=C\[C@H](O)[C@@H](O)C/C=C\CCC(=O)O]

and pharmaceutically acceptable salts and esters thereof.

Other compounds suitable for use in methods of the invention include those of Formula 7.

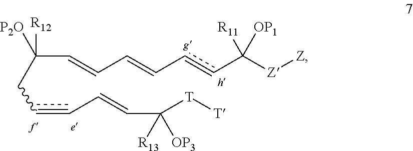

or pharmaceutically acceptable salts thereof, wherein
Carbons e′ and f′ are connected by a double bond or a triple bond, and when carbon e′ is connected to carbon f′ through a double bond the stereochemistry is cis or trans;
Carbons g′ and h′ are connected by a double bond or a triple bond and when carbon g′ is connected to carbon h′ through a double bond the stereochemistry is cis or trans;
m is 0 or 1;
T′ is hydrogen, (C1-C6) alkyl, (C2-C6) alkenyl, (C2-C6) alkynyl, (C5-C14) aryl, (C6-C16) arylalkyl, 5-14 membered heteroaryl, 6-16 membered heteroarylalkyl, or —CH═CHCH₂CH₃;
T is —(CH₂)_{q}— or —(CH₂)_{q}—O—, where q is an integer from 0 to 6;
Z′ is (C1-C6) alkylene optionally substituted with 1, 2, 3, 4, 5 or 6 of the same or different halogen atoms, —CH₂)ₚ—O—CH₂— or —(CH₂)ₘ—S—CH₂—, where p is an integer from 0 to 4;
R₁₁, R₁₂ and R₁₃ each individually is substituted or unsubstituted, branched or unbranched alkyl, alkenyl, or alkynyl group, substituted or unsubstituted aryl group, substituted or unsubstituted, branched or unbranched alkylaryl group. C₁₋₄alkoxy, halogen atom, —CH₂R₁₄, —CHR₁₄R₁₄, —CR₁₄R₁₄R₁₄, or a hydrogen atom;
R₁₄ is independently for each occurrence selected from —CN, —NO₂ or halogen;
P₁, P₂, P₃, and Z are as defined above.

Other compounds suitable for use in methods of the invention include those of Formula 8.

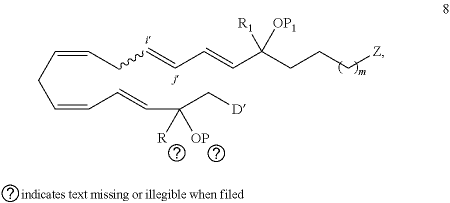

or pharmaceutically acceptable salts thereof, wherein
the stereochemistry of the carbon i′ to carbon j′ bond is cis or trans;
m is 0 or 1;
D′ is CH₃, —CH═CHCH₂U or —CH═CHCH₂CH₂A;
U is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
A is H or —OP₄;
P₁, P₂, P₄, R₁, R₂ and Z are as defined above.

Other compounds suitable for use in methods of the invention include those of Formula 9.

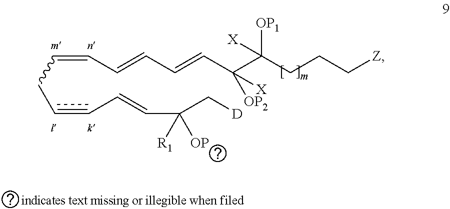

or pharmaceutically acceptable salts thereof, wherein
Carbons k′ and l′ are connected by a double bond or a triple bond;
the stereochemistry of the carbon m′ to carbon n′ double bond is cis or trans;
m is 0 or 1;
D is —CH₃ or —CH═CHCH₂CH₃;
P₁, P₂, P₃, R₁, X, and Z are as defined above.

Exemplary compounds of formula 9 include compound 9a,

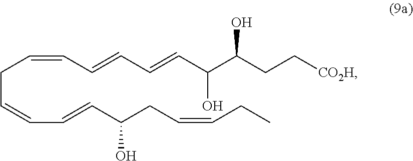 [SMILES: CC/C=C\C[C@H](O)/C=C/C=C\C\C=C/C=C/C=C/C(O)[C@@H](O)CCC(=O)O]

and pharmaceutically acceptable salts and esters thereof.

Other compounds suitable for use in methods of the invention include those of Formula 10,

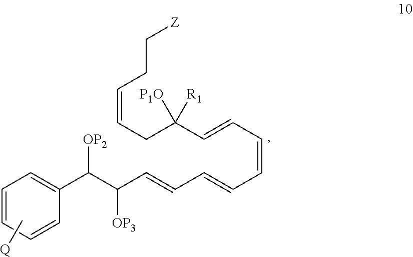

or pharmaceutically acceptable salts thereof, wherein
P₁, P₂, P₃, R₁ and Z are as defined above; and
Q represents one or more substituents and each Q individually, is present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;

Other compounds suitable for use in methods of the invention include those of Formula 11,

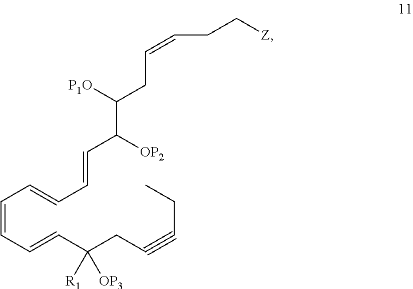

or pharmaceutically acceptable salts thereof, wherein
P₁, P₂, P₃, R₁, and Z are as defined above.

Other compounds suitable for use in methods of the invention include those of Formula 12,

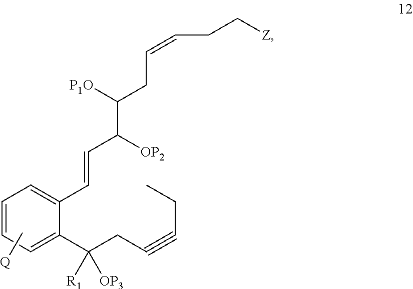

or pharmaceutically acceptable salts thereof, wherein
P₁, P₂, P₃, Q, R₁, and Z are as defined above.

Other compounds suitable for use in methods of the invention include those of Formula 13,

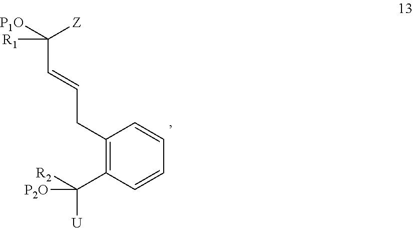

or pharmaceutically acceptable salts thereof, wherein
P₁, P₂, R₁, R₂, U, and Z are as defined above.

Other compounds suitable for use in methods of the invention include those of Formula 14,

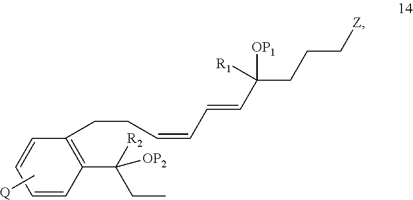

or pharmaceutically acceptable salts thereof, wherein
P₁, P₂, R₁, R₂, Q, and Z are as defined above.

Other compounds suitable for use in methods of the invention include those of Formula 15,

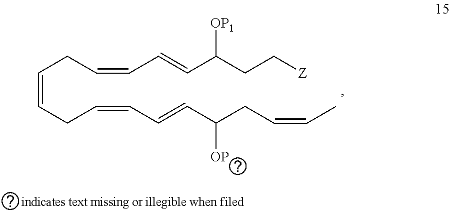

or pharmaceutically acceptable salts thereof, wherein
P₁, P₂, and Z are as defined above.

Other compounds suitable for use in methods of the invention include those of Formula 16.

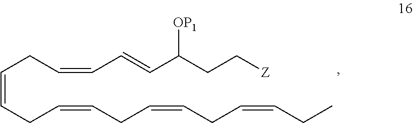

or pharmaceutically acceptable salts thereof, wherein
P₁ and Z are as defined above.

Other compounds suitable for use in methods of the invention include those of Formula 17.

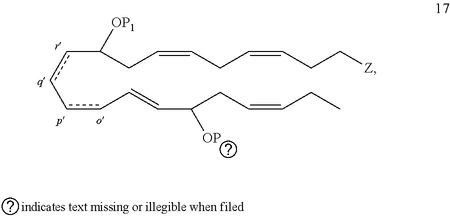

or pharmaceutically acceptable salts thereof, wherein
Carbons o′ and p′ are connected by a single or a double bond;
Carbons q′ and r′ are connected by a single or a double bond; and
P₃, P₂, and Z are as defined above.

Other compounds suitable for use in methods of the invention include those of Formula 18.

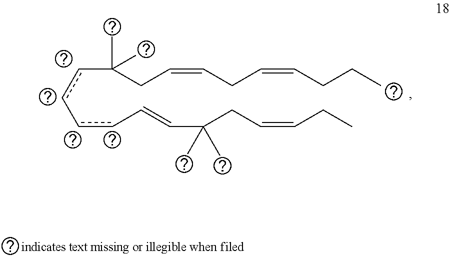

or pharmaceutically acceptable salts thereof, wherein
the stereochemistry of the carbon s′ to carbon t′ double bond is cis or trans;
the stereochemistry of the carbon u′ to carbon v′ double bond is cis or trans; and
P₁, P₂, R₁, R₂, and Z are as defined above.

Other compounds suitable for use in methods of the invention include those of Formula 19,

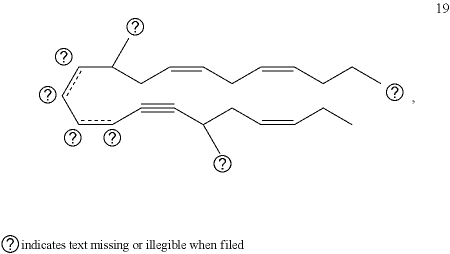

or pharmaceutically acceptable salts thereof, wherein
Carbons w′ and x′ are connected by a single or a double bond;
Carbons y′ and z′ are connected by a single or a double bond; and
P₁, P₂, and Z are as defined above.

In certain embodiments of formulae 4 to 19, each R^{b}, Group independently selected form ═O, —OR^{d}, (C1-C3) haloalkyloxy, —OCF₃, ═S, —SR^{d}═NR^{d}, ═NOR^{d}, —NR^{c}R^{c}, halogen, —CF₃, —CN, —NC, —OCN, —OCN, —SCN, —NO, —NO₂, ═N₂, —N₃—S(OR)R^{d}, —S(O)₂R^{d}, —S(O)₂R^{d}, —S(O)₂NR^{c}R^{c}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{a}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{d}R^{c}, −[NHC(O)]ₐR^{d}, —[NR^{a}C(O)]ₛR^{d}, —[NHC(O)]ₙOR^{d}, [NHC(O)]₆NR^{c}R^{c}, —[NR^{a}C(O)]ₐNR^{c}R^{c}, —[NHC(NH)]⁸NR^{c}R^{c} and —[NR^{a}C(NR^{a})]₆NR^{c}R^{c}.

Other compounds suitable for use in methods of the invention include those of Formula 20,

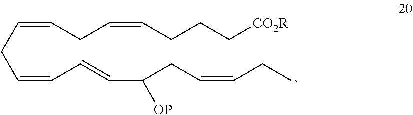

Formula 21,

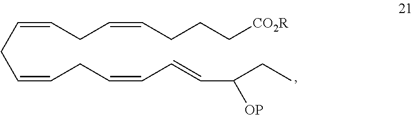

Formula 22,

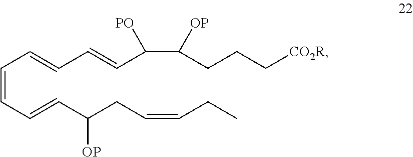

Formula 23,

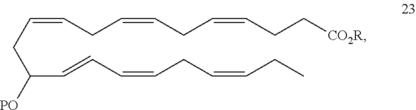

Formula 24,

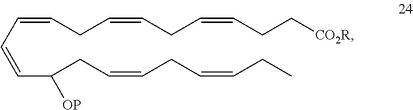

Formula 25,

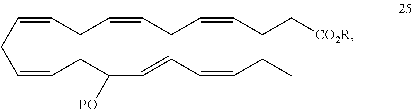

Formula 26,

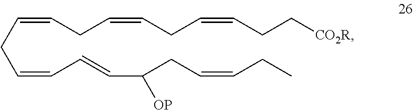

Formula 27,

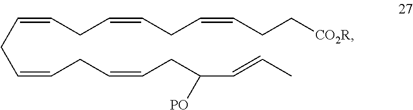

or Formula 28,

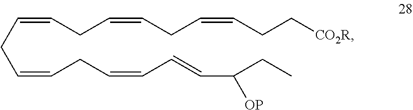

or pharmaceutically acceptable salts of any of the above, wherein
each P is individually selected from H or a protecting group; and
R is H, C₁₋₆alkyl (e.g., methyl, ethyl, glycerol), C₂₋₆alkenyl or C₂₋₆alkynyl,

Other compounds suitable for use in methods of the invention include those of Formula 29.

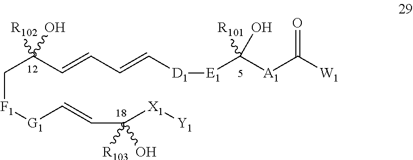

and pharmaceutically acceptable salts, hydrates and solvates thereof, wherein:
D₁E₁ and F₁-G₁ are independently are cis or trans —C═C— or —C≡C—:
R₁₀₁, R₁₀₂ and R₁₀₃ are independently selected from hydrogen, (C1-C4) straight-chained or branched alkyl, (C2-C4) alkenyl, (C2-C4) alkynyl, (C1-C4) alkoxy, —CH₂R₁₀₄, —CHR₁₀₄R₁₀₄ and —CR₁₀₄R₁₀₄R₁₀₄;
each R₁₀₄ is independently selected from CN, —NO₂ and halogen;
W₁ is selected from —R₁₀₅, —OR₁₀₅, —SR₁₀₅ and —NR₁₀₅R₁₀₅;
each R₁₀₅ is independently selected from hydrogen, (C1-C6) alkyl, (C2-C6) alkenyl or (C2-C6) alkynyl optionally substituted with one or more of the same or different R groups, (C5-C14) aryl optionally substituted with one or more of the same or different R groups, phenyl optionally substituted with one or more of the same or different R groups, (C6-C16) arylalkyl optionally substituted with one or more of the same of different R groups, 5-14 membered heteroaryl optionally substituted with ne or more of the same or different R groups, 6-16 membered heteroarylalkyl optionally substituted with one or more of the same or different R groups and a detectable label molecule;
A₁ is selected from (C1-C6) alkylene optionally substituted with 1, 2, 3, 4, 5 or 6 of the same or different halogen atoms, —(CH₂)ₘ—O—CH₂— and —(CH₂)ₘ—S—CH₂—, where m is an integer from 0 to 4;
X₁ is selected from —(CH₂)ₙ— and —(CH₂)ₙ—O—, where n is an integer from 0 to 6;
Y₁ is selected from hydrogen, (C1-C6) alkyl, (C2-C6) alkenyl or (C2-C6) alkynyl, optionally substituted with one or more of the same or different R₁₀₀ groups, (C5-C14) aryl optionally substituted with one or more of the same or different R₁₀₀ groups, phenyl optionally substituted with one or more of the same or different R₁₀₀ groups, (C6-C16) arylalkyl optionally substituted with one or more of the same of different R₁₀₀ groups, 5-14 membered heteroaryl optionally substituted with one or more of the same or different R₁₀₀ groups, 6-16 membered heteroarylalkyl optionally substituted with one or more of the same or different R₁₀₀ groups and a detectable label molecule;
each R₁₀₀ is independently selected from an electronegative group, ═O, —OR^{a3}, (C1-C3) haloalkyloxy, ═S, —SR^{a1}, ═NR^{a1}, ═NONR^{a1}, —NR^{c1}R^{c1}, halogen, —CF₃, —CN, —NC, —OCN, —SCN, —NO, —NO₂, ═N₂, —N₃, —S(OR)R^{a1}, —S(O)₂R^{a1}, —S(O)₂OR^{a1}, —S(O)₂NR^{c1}R^{c1}, —OS(O)R^{a1}, —OS(O)₂OR^{a1}, —OS(O)NR^{a1}R^{c1}, —C(O)₂R^{a1}, —C(O)ORₐ₁, —C(O)NR_{c1}R^{c1}, —C(NH)NR^{c1}R^{c1}, —OC(O)R^{a1}, —OC(O)OR^{a1}, —OC(O)NR^{c1}R^{c1}, —OC(NH)NR^{c1}R^{c1}, —NHC(O)R^{a1}, —NHC(O)OR^{a1}, —NHC(O)NR^{c1}R^{c1} and —NHC(NH)NR^{c1}R^{c1};
each R^{a1} is independently selected from hydrogen, (C1-C4) alky, (C2-C4) alkenyl or (C2-C4) alkynyl; and each R^{c1} is independently and R^{a1} or, alternatively, R^{c1}R^{c1} taken together with the nitrogen atom to which it is bonded forms a 5 or 6 membered ring.

In certain embodiments of Formula 29, when X₁-Y₁ is —CH₂CH₃, then at least one of R₁₀₁, R₁₀₂ or R₁₀₃ is other than hydrogen.

In certain embodiments, a compound of Formula 29 is represented by Formula 30,

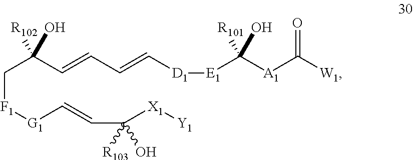

Other compounds suitable for use in methods of the invention include those of Formulae 31 to 37

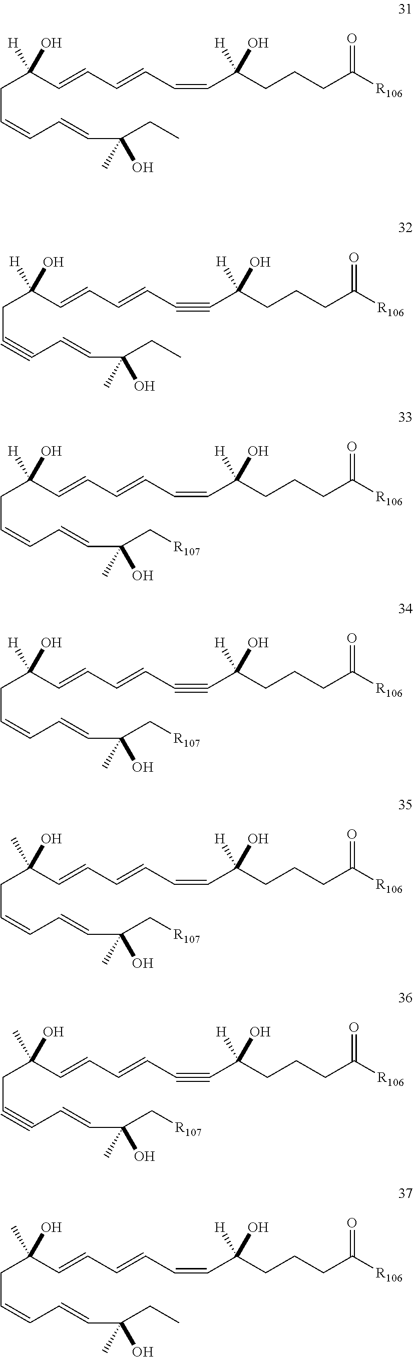

and pharmaceutically acceptable salts, hydrates and solvates thereof, wherein
R₁₀₇ is —OH, —OCH₃, —OCH(CH₃)₂ —NHCH₂CH₃; and
R₁₀₇ is

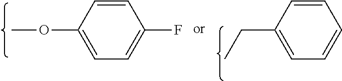

Other compounds suitable for use in methods of the invention include those of Formula 38,

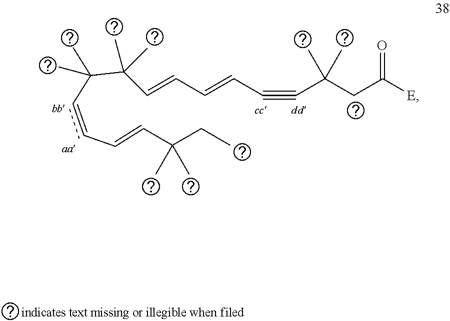

wherein
Carbons aa′ and bb′ are connected by a double bond or a triple bond;
Carbons cc′ and dd′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
E is hydroxyl, alkoxy, aryloxy, amino, alkylamino, diakylamino, or arylamino;
Rh, Ri and Rj are independently selected from hydrogen, alkyl, alkenyl, alkynyl, perfluoroalkyl, aryl or heteroaryl;
R₄ is selected from hydrogen, alkyl, perfluoroalkyl, alkenyl, alkynyl, aryl, heteroaryl, fluoro, hydroxyl, alkoxy, aryloxy;
R₆ is selected from i-iv as follows: i) CH₂CH(R₆)CH₂, where R₆ is hydrogen, alkyl, alkenyl, alkynyl, perfluoroalkyl, aryl, heteroaryl, fluoro, hydroxyl or alkoxy; ii) CH₂C(R₆R₇)CH₂, where R₆ and R₇ are each independently alkyl, alkenyl, alkynyl, perfluoroalkyl, aryl, or fluoro, or R₆ and R₇ are connected together to form a carbocyclic or heterocyclic ring; iii) CH₂OCH₂, CH₂C(O)CH₂, or CH₂CH₂; or iv) R₅ is a carbocyclic, heterocyclic, aryl or heteroaryl ring; and
R₈ R₉ are independently selected from hydrogen, alkyl, alkenyl, alkynyl, perfluoroalkyl, alkoxy, aryl or heteroaryl, or R₈ and R₉ are connected together to form a carbocyclic or heterocyclic ring;
   or pharmaceutically acceptable salts thereof.

In certain embodiment R₈ and R₉ are hydrogen.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Other compounds suitable for use in methods of the invention include those of Formulae 39-44,

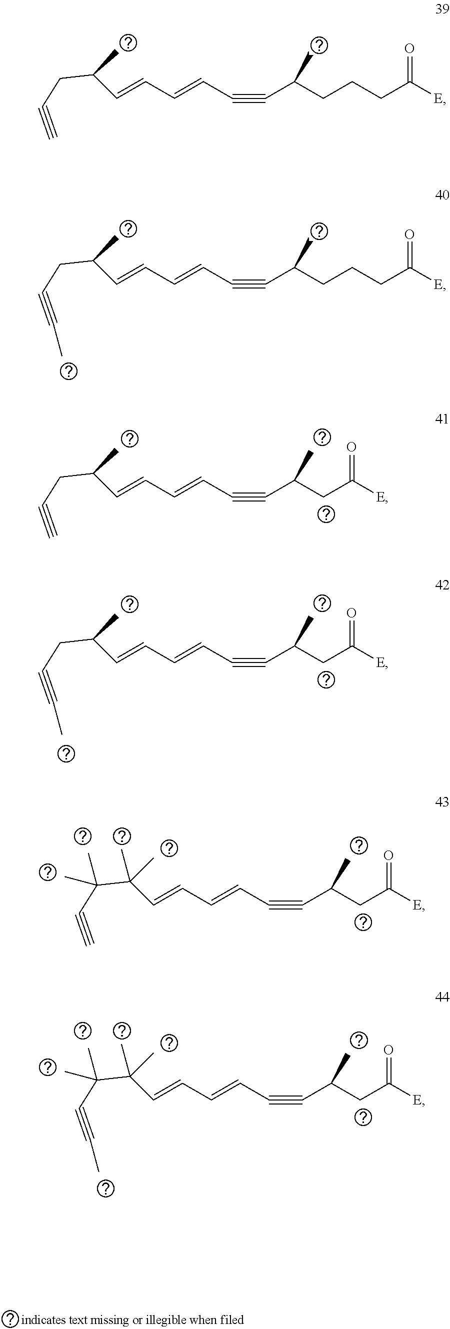

and pharmaceutically acceptable salts thereof, wherein
Re, Rf, E, Ri, R₅, R₈ and R₉ are as defined above.

Exemplary compounds of formulae 38, 41, and 43 include:

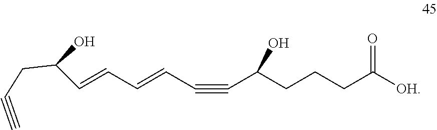 [SMILES: C#CC[C@@H](O)/C=C/C=C/C#C[C@@H](O)CCCC(=O)O]

In certain embodiments, a pharmaceutically acceptable salt of the compound is formed by derivatizing E, wherein E is —OM, where M is a cation selected from ammonium, tetra-alkyl ammonium, Na, K, Mg, and Zn. Examples of such compounds include compound Z,

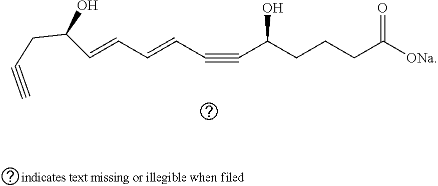

Other compounds suitable for use in methods of the invention include those of Formula 46,

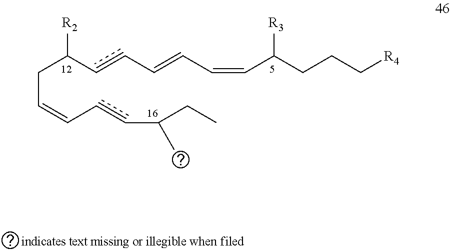

or a pharmaceutically acceptable salt or prodrug thereof, wherein:
each [symbol] independently designates a double or triple bond;
R¹, R², and R³ are each independently OR, OX¹, SR, SX², N(R)₂, NHX³, NRC(O)R, NRC(O) N(R)₂, C(O)OR, C(O)N(R)₂, SO₂R, NRSO_{X}R, CO(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or;
two R n the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O), NRC(O)N(R)₂, C(O)OR, C(O)N(R)₂, SO₂R, NRSO₂R, C(O)R, or SO₂N(R)₂.

Other compounds suitable for use in methods of the invention include those of Formula 47;

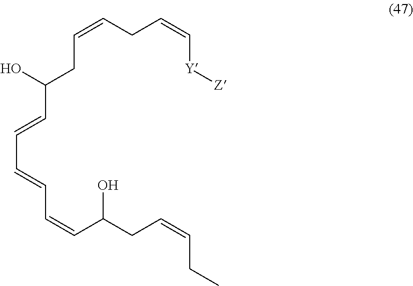

or a pharmaceutically acceptable salt or prodrug thereof, wherein;
Y′ is a bond or a linker selected from a ring containing up to 20 atoms or a chain of up to 20 atoms, provided that Y′ can include one or more nitrogen, oxygen, sulfur or phosphorous atoms, further provided that Y′ can include one or more substituents independently selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkythio, arylthio, acylthio, alkylsulfonate, arylsulfonate, phophoryl, or sulfonyl, further provided that Y′ can contain one or more fused carbocyclic, heterocyclic, aryl or heteroaryl rings;
Z′ is selected from —CN, —C(NH)N(R″)(R″), —C(S)—A′, —C(S)R″, —C(O)—A′, —C(O)—R″, —C(O)—SR″, —C(O)—NH—S(O)₂R″, —S(O)₂—A′, —S(O)₂—R″, S(O)₂N(R″)(R″), —P(O)₂—A′, —PO(OR″)—A′, -tetrazole, alkyltetrazole, or —CH₂H, wherein A′ is selected from —OR″, —N(R″)(R″) or —OM′; each R″ is independently selected from hydrogen, alkyl, aryl, arylalkyl, heteroaryl, heteroarylalkyl or a detectable label molecule, wherein any alkyl- , aryl- or heteroaryl-containing moiety is optionally substituted with up to 3 independently selected substituents; and M′ is a cation.

In certain embodiments, a compound of formula 47 is represented by formula

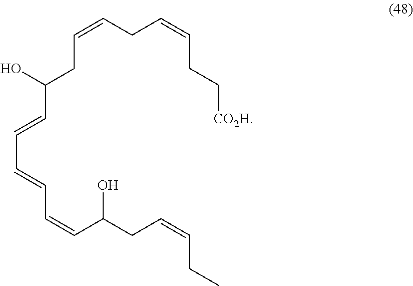 [SMILES: CC/C=C\CC(O)\C=C/C=C/C=C/C(O)C/C=C\C/C=C\CCC(=O)O]

In certain embodiments, a compound of formula 47 is represented by formula

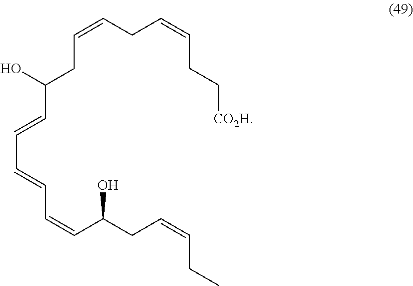 [SMILES: CC/C=C\C[C@H](O)\C=C/C=C/C=C/C(O)C/C=C\C/C=C\CCC(=O)O]

The compounds above (e.g., compounds of formula A or formulae 1 to 49) are known to be useful in the treatment or prevention of inflammation or inflammatory disease. Examples of such compounds are disclosed in the following patents and applications: US 2003/0191184, WO 2004/014835, WO 2004/078143, U.S. Pat. No. 6,670,396, US 2003/0236423, US 2005/0228047, US 2005/0238589 and US2005/0261255. These compounds are suitable for use in methods of the present invention.

Other compounds useful in this invention are compounds that are chemically similar variants to any of the compounds of formula A or formulae 1-49 or I-III set forth above. The term “chemically similar variants” includes, but is not limited to, replacement of various moieties with known biosteres; replacement of the end groups of one of the compounds above with a corresponding end group of any other compound above, modification of the orientation of any double bond in a compound, the replacement of any double bond with a triple bond in any compound, and the replacement of one or more substituents present in one of the compounds above with a corresponding substituent of any other compound.

Lipoxin compounds suitable for use in this invention include those of formula 50:

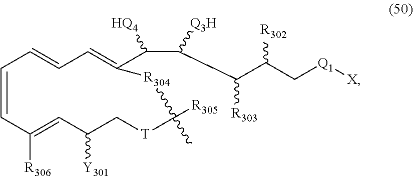

wherein:
X is R₃₀₁, OR₃₀₁, or SR₃₀₁;
R₃₀₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;

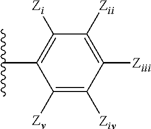

(f) substituted phenyl wherein Z_{I} Zᵢᵢ, Zᵢᵢᵢ, Zᵢᵥ and Zᵥ are each independently selected from —NO₂, —CN, —C(═O)—R₃₀₁, —SO₃H, a hydrogen atom, halogen, methyl, —ORₓ, wherein R₈ is 1 to 8 carbon atoms, inclusive, which may be a straight chain or branched, any hydroxyl, wherein when any of Z_{i Z}ᵢᵢ, Zᵢᵢᵢ, Zᵢᵥ or Zᵥ is C(═O)—R₃₀₁, said Z_{i Z}ᵢᵢZᵢᵢᵢ, Z_{iv or Z}ᵥ is not substituted with another C(═O)—R₃₀₁;
(g) a detectable label molecule; or
(h) a straight or branched chain alkenyl of 2 to 8 carbon atoms, inclusive;
Q₁ is (C═O), SO₂ or (CN), provided when Z₁ is CN, then X is absent; Q₃ and Q₄ are each independently O, S or NH;
one of R₃₀₂ and R₃₀₃ is a hydrogen atom and the other is:
   (a) H;
   (b) an alkyl of 1 to 8 carbon atoms, inclusive, which may be a straight chain or branched;
   (c) a cycloalkyl of 3 to 6 carbon atoms, inclusive;
   (d) an alkenyl of 2 to 8 carbon atoms, inclusive, which may be straight chain or branched; or
   (e) RₖQ₂R₈ wherein Q₂ is —O— or —S—; wherein Rₖ is alkylene of 0 to 6 carbons atoms, inclusive, which may be straight chain or branched and wherein R₁ is alkyl of 0 to 8 carbon atoms, inclusive, which may be straight chain or branched, provided when R₁ is 0, then R₁ is a hydrogen atom;
   R₃₀₄ is
   (a) H;
   (b) an alkyl of 1 to 6 carbon atoms, inclusive, which may be a straight chain or branched;
   R₃₀₅ is

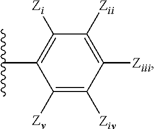

wherein Zᵢ Zᵢᵢ, Zᵢᵢᵢ, Zᵢᵥ and Zᵥ are defined as above;
R₃₀₅ is
(a) H;
(b) an alkyl from 1 to 4 carbon atoms, inclusive, straight chain or branched;
wherein Y₃₀₁ is —OH, methyl, —SR, an alkyl of 2 to 4 carbon atoms, inclusive, straight chain or branched, an alkoxy of 1 to 4 carbon atoms, inclusive, or (CH)_{g}(Z)_{q}, where p+q=0 to 3, q=0 to 3 and Z is cyano, nitro or a halogen; and
T is O or S, and pharmaceutically acceptable salts thereof.

Lipoxin compounds suitable for use in this invention include those of Formulae 51, 52, 53 or 54:

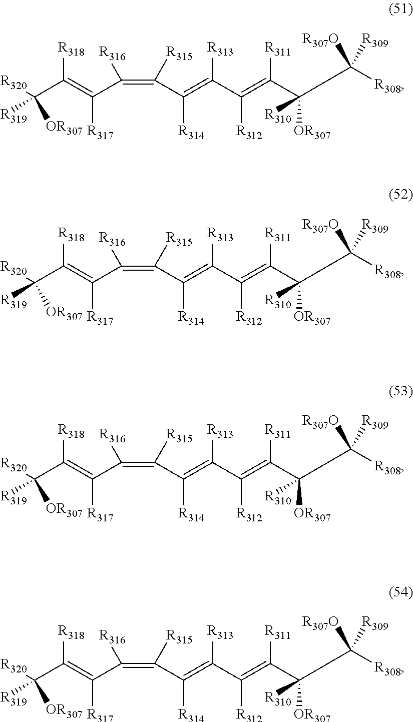

wherein:
each R₃₀₇ is independently selected from hydrogen and straight, branched, cyclic, saturated, or unsaturated alkyl having from 1 to 20 carbon atoms;
R₃₀₈, R₃₀₉, R₃₁₀, R₃₁₉, and R₃₂₀ are independently selected from;
(a) hydrogen;
(b) straight, branched, cyclic, saturated, or unsaturated alkyl having from 1 to 20 carbon atoms;
(c) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, caboalkoxy, aryl, and heteroaryl;
(d) substituted aryl or heteroaryl wherein the aryl or heteroaryl is substituted with one or more substituents selected from alkyl, cycloalkyl, alkoxy, halo, aryl, heteroaryl, carboxyl, and carboxamido; and
(e) Z—Y, wherein:
   Z is selected from a straight, branched, cyclic, saturated, or unsaturated alkyl having from 1 to 20 carbon atoms; substituted lower alkyl, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; and substituted aryl or heteroaryl, wherein the aryl or heteroaryl is substituted with ne or more substituents selected from alkyl, cycloalkyl, alkoxy, halo, aryl, heteroaryl, carboxyl, and carboxamido; and
   Y is selected from hydrogen; alkyl; cycloalkyl; carboxyl; carboxamido; aryl; heteroaryl; substituted aryl or heteroaryl, wherein the aryl or heteroaryl is substituted with one or more substituents selected from alkyl, cycloalkyl, alkoxy, halo, aryl, heteroaryl, carboxyl, and carboxamido; and
   R₃₁₁ to R₃₁₈ are independently selected from;
(a) hydrogen;
(b) halo;
(c) straight, branched, cyclic, saturated, or unsaturated alkyl having from 1 to 20 carbon atoms;
(d) substituted alkyl having from 1 to 20 carbon atoms, wherein the alkyl is substituted with ne or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carbalkoxy, aryl, and heteroaryl;
(e) substituted aryl or heteroaryl, wherein the aryl or heteroaryl is substituted with one or more substituents selected from alkyl, cycloalkyl, alkoxy, halo, aryl, heteroaryl, carboxyl, and carboxamido; or
R₃₀₈ to R₃₂₀ are independently a bond that forms a carbon-carbon double bond, a carbon-carbon triple bond, or a ring with the lipoxin backbone; or
any two of R₃₀₇ to R₃₂₆ are taken together with the atoms to which they are bound and optionally to 1 to 6 oxygen atoms, 1 to 6 nitrogen atoms, or both 1 to 6 oxygen atoms and 1 to 6 nitrogen atoms, to form a ring containing 3 to 20 atoms.

Lipoxin compounds suitable for use in this invention include those of formula 55:

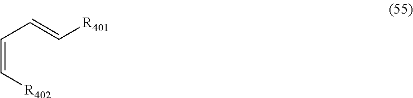

wherein
R₄₀₁ is selected from:

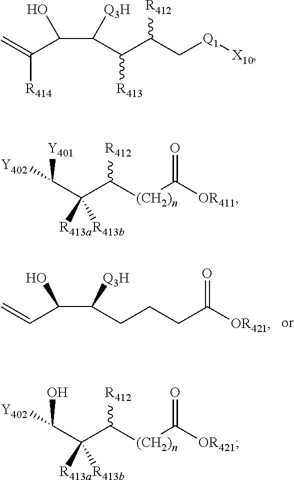

R₄₀₂ is selected from:

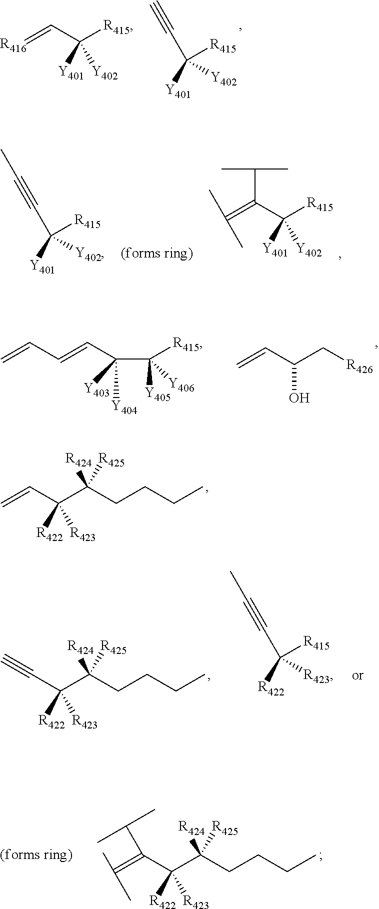

X₁₀ is R₄₁₁, OR₄₁₁, or SR₄₁₁;
R₄₁₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;

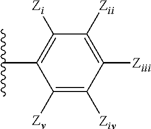

(f) substituted phenyl wherein Zᵢ , Zᵢᵢ, Zᵢᵢᵢ, Zᵢᵥ and Zᵥ are each independently selected from —NO₂, —CN, —C(═O)—R₄₁₁, —SO₃H, a hydrogen atom, halogen, methyl, —OR, wherein Rₓ is 1 to 8 carbon atoms, inclusive, which may be a straight chain or branched, and hydroxyl; wherein when any of Zᵢ, Zᵢᵢ, Zᵢᵢᵢ, Zᵢᵥ Zᵥ is C(═O)—R₄₁₁, said Zᵢ Zᵢᵢ, Zᵢᵢᵢ, Zᵢᵥ or Zᵥ is not substituted with another C(═O)—R₄₁₁.
(g) a detectable label molecule; or
(h) a straight or branched chain alkenyl of 2 to 8 carbon atoms, inclusive;

Q₁ is (C═O), SO₂ or (CN);

Q₃ is O, S or NH;
one of R₄₁₂ and R₄₁₃ is a hydrogen atom and the other is selected from:
   (a) H;
   (b) an alkyl of 1 to 8 carbon atoms, inclusive, which can be straight chain or branched;
   (c) a cycloalky of 3 to 6 carbon atoms, inclusive:
   (d) an alkenyl of 2 to 8 carbon atoms, inclusive, which can be straight chain or branched; or
   (e) R₄₃₁Q₂R₄₃₂ wherein Q₂ is —O— or —S—; wherein R₄₃₁ is alkylene of 0 to 6 carbons atoms, inclusive, which can be straight chain or branched and wherein R₄₃₁ is alkyl of 0 to 8 carbon atoms, inclusive, which can be straight chain or branched;

R₄₁₃ₐ, and R_{413b} are each independently:
(a) H;
(b) an alkyl of 1 to 8 carbons, inclusive, which can be straight chain or branched;
(c) a cycloalkyl of 3 to 6 carbon atoms, inclusive:
(d) an alkenyl of 2 to 8 carbon atoms, inclusive, which can be straight chain or branched; or
(e) R₄₃₁Q₂R₄₃₂ wherein R₄₃₁, Q₂ , and R₄₃₂ are as defined above;
R₄₁₄ is
   (a) H;
   (b) an alkyl of 1 to 6 carbon atoms, inclusive, can be straight chain or branched;
R₄₁₅ is
   (a) an alkyl of 1 to 9 carbon atoms which can be straight chain or branched;
   (b) —(CH₂)—R₁
wherein n=0 to 4 and R₁ is
(i) a cycloalkyl of 3 to 10 carbon atoms, inclusive;
   (ii) a phenyl; or
   (iii) substituted phenyl

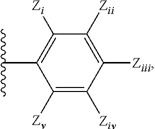

wherein Zᵢ though Zᵥ are as defined above;
(R₄₃₁Q₂R₄₃₂, wherein R₄₃₁, Q₂, and R₄₃₂ are as defined above;
(c) —C(Rᵢᵢᵢ)(Rᵢᵥ)—Rᵢ.
wherein Rᵢᵢᵢ and Rᵢᵥ are each independently:
   (i) a hydrogen atom;
   (ii) (CH)ₚ(Z)_{q}, wherein Z, p , and q are as defined above;
(e) a haloalkyl of 1 to 8 carbon atoms, inclusive, and 1 to 6 halogen atoms, inclusive, straight chain or branched;
R₄₁₆ is
   (a) H;
   (b) an alkyl from 1 to 4 carbon atoms, inclusive, straight chain or branched;
   (c) a halogen;

one of Y₄₀₁ or Y₄₀₂ is —OH, methyl, or —SH, and wherein the other is selected from:
(a) H;
(b) (CH)ₚ(Z)_{q} wherein p+q=3, p=0 to 3, q=0 to 3 and each Z, independently, is cyano, nitro or a halogen;
(c) an alky of 2 to 4 carbon atoms, inclusive, straight chain or branched; or
(d) an alkoxy of 1 to 4 carbon atoms, inclusive, or Y₄₀₁ and Y₄₀₂ taken together are:
(d) ═NH; or
(e) ′O;

one of Y₄₀₃ or Y₄₀₄ is —OH, methyl, or —SH, and wherein the other is selected from:
(a) H;
(b) (CH)ₚ(Z)_{q} wherein Z, p, and q are as defined above;
(c) an alkyl of 2 to 4 carbon atoms, inclusive, straight chain or branched; or
(d) an alkoxy of 1 to 4 carbon atoms, inclusive.

or Y₄₀₁ and Y₄₀₂ taken together are:
(a) ═NH; or
(b) ═O;

one of Y₄₀₅ or Y₄₀₆ is —OH, methyl, or —SH, and wherein the other is selected from:
(a) H
(b) (CH)ₚ(Z)_{q} wherein Z, p, and q are as defined above; (p1 (c) an alkyl of 2 to 4 carbon atoms, inclusive, straight chain or branched; or
(d) an alkoxy of 1 to 4 carbon atoms, inclusive, Or Y₄₀₁ and Y₄₀₂ taken together are:
(a) ═NH; or
(b) ═O;

R₄₂₁
(a) H; or
(b) alky of 1 to 8 carbon atoms;

R₄₂₂ and R₄₂₃ are each independently:
(a) H;
(b) a hydroxyl, or a thiol;
(c) a methyl or a halomethyl;
(d) a halogen or
(e) an alkoxy of 1 to 3 carbon atoms;

R₄₂₄ and R₄₂₅ are each independently;
(a) H;
(b) a hydroxyl, or a thiol;
(c) a methyl or a halomethyl;
(d) a halogen;
(e) an alkoxy of 1 to 3 carbon atoms; or
(f) an alkyl or haloalkyl of 2 to 4 carbon atoms inclusive, which can be straight chain or branched; and

R₄₂₆ is
(a) a substituted phenyl

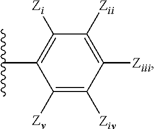

wherein Zᵢ through Zᵥ are as defined above
(b) a substituted phenoxy

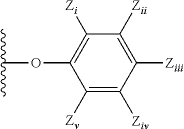

wherein Zᵢ through Zᵥ are as defined above; or (c)

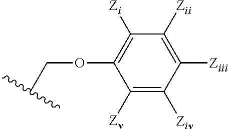

wherein Zᵢ through Zᵥ are as defined above.

Lipoxin compounds suitable for use in this invention included those of formula 56:

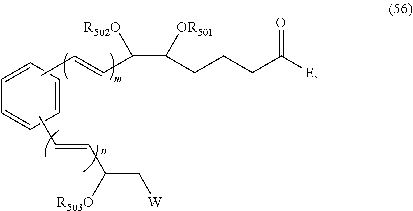

wherein:
E is hydrogen, alkoxy, amino, alkylamino, dialkylamino or —OM, where M is a cation selected from ammonium, tetra-alkyl ammonium, and the cations of sodium, potassium, magnesium and zinc;
W is hydrogen, alkyl, alkenyl, alkynyl, aryl, heteroaryl, halo, hydroxy, alkoxy, aryloxy, carboxy, amino, alkylamino, dialkylamino, acylamino, carboxamido, or sulfonamide;
each of R₅₀₁-R₅₀₃ are independently selected from hydrogen, alkyl, aryl, acyl or alkoxyacyl;
n is 0, 1 or 2;
m is 1 or 2; and
the two substituents on the phenyl ring are ortho, meta, or para.

Lipoxin compounds suitable for use in this invention include those of formula 57:

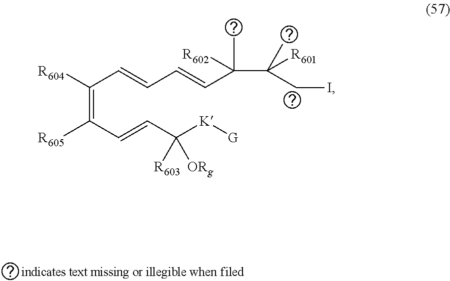

wherein:
I is selected from: —C(O)—E, —SO₂—E, —PO(OR)—E, where E is hydroxy, alkoxy, aryloxy, amino, alkylamino, dialkylamino, or —OM, where M is a cation selected from ammonium, tetra-alkyl ammonium. Na, K, Mg, and Zn: and R is hydroxyl or alkoxy
J′ and K′ are linkers independently selected from a chain of up to 20 atoms and a ring containing up to 20 atoms, provided that J′ and k′ can independently include one or more nitrogen, oxygen, sulfur or phosphorous atoms, and further provided that J′ and K′ can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphorly, or sulfonyl, further provided that J′ and K′ can also contain one or more fused carbocyclic, heterocyclic, aryl or heteroaryl rings, and provided that linkers J′ and K′ are connected to the adjacent C(R)OR group via a carbon atom or a C-heteroatom bond where the heteroatom is oxygen, sulfur, phosphorous or nitrogen;
G selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.
Re, Rf and Rg, are independently selected from hydrogen, alkyl, aryl, heteroaryl, acyl, silyl, alkoxyacyl and aminoacyl;
R₆₀₁, R₆₀₂ and R₆₀₃ are independently selected from hydrogen, alkyl, aryl and heteroaryl, provided that R₆₀₁, R₆₀₂ and R₆₀₃ can independently be connected to linkers J′ or K′;
   R₆₀₂ and R₆₀₅ are independently selected from hydrogen, alkyl, alkenyl, alkynyl, aryl, heteroaryl, fluoro, and provided that R₆₀₄ and R₆₀₅ can be joined together to form a carbocyclic, heterocyclic or aromatic ring, and further provided that R₆₀₄ and R₆₆₅ can be replaced by a bond to form a triple bond.

Other compounds suitable for use in methods of the invention are the oxylipins described in international applications WO 2006055965, WO 2007090162, and WO2008103753, the compounds in which are incorporated herein by reference. Examples of such compounds are those of formulae 58-132, as shown in Table 1. These compounds include long chain omega-6 fatty acids, docosapentaenoic acid (DPAn-6) (compounds 58-73) and docosatetraenoic acid (DTAn-6) (compounds 74-83), and the omega-3 counterpart of DPAn-6, docosapentaenoic acid (DPAn-3) (compounds 84-97). Further compounds are the docosanoids 98-115, the γ-linolenic acids (GLA) (compounds 116-122), and the stearidonic acids (SDA) (compounds 123-143).

TABLE 1

| | |
|---|---|
| 10,17-Dihydroxy DPAn-6 (58) | 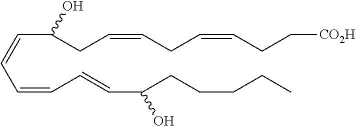 [SMILES: CCCCCC(O)/C=C/C=C\C=C/C(O)C/C=C\C/C=C\CCC(=O)O] |
| 16,17-Dihydroxy DPAn-6 (59) | 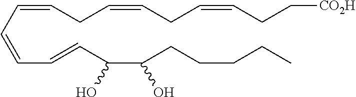 [SMILES: CCCCCC(O)C(O)/C=C/C=C\C=C/C/C=C\C/C=C\CCC(=O)O] |
| 4,5-Dihydroxy DPAn-6 (60) | 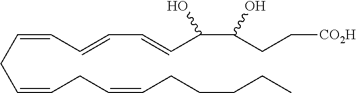 [SMILES: CCCCC/C=C\C/C=C\C\C=C/C=C/C=C/C(O)C(O)CCC(=O)O] |
| 7,17-Dihydroxy DPAn-6 (61) | 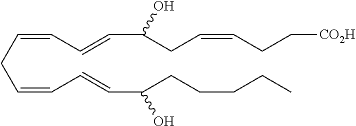 [SMILES: CCCCCC(O)/C=C/C=C\C/C=C\C=C\C(O)C/C=C\CCC(=O)O] |
| 7-Hydroxy DPAn-6 (62) | 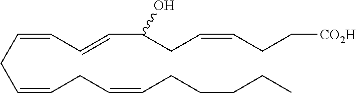 [SMILES: CCCCC/C=C\C/C=C\C/C=C\C=C\C(O)C/C=C\CCC(=O)O] |
| 10-hydroxy DPAn-6 (63) | 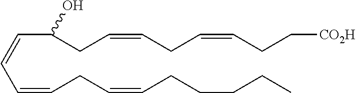 [SMILES: CCCCC/C=C\C/C=C\C=C/C(O)C/C=C\C/C=C\CCC(=O)O] |
| 13-Hydroxy DPAn-6 (64) | 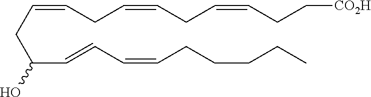 [SMILES: CCCCC/C=C\C=C\C(O)C/C=C\C/C=C\C/C=C\CCC(=O)O] |
| 17-hydroxy DPAn-6 (65) | 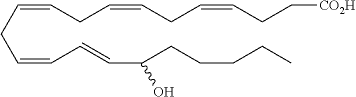 [SMILES: CCCCCC(O)/C=C/C=C\C/C=C\C/C=C\C/C=C\CCC(=O)O] |
| 4,5,17-Trihydroxy DPAn-6 (66) | 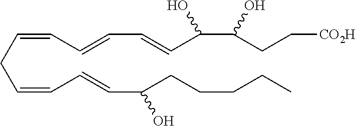 [SMILES: CCCCCC(O)/C=C/C=C\C\C=C/C=C/C=C/C(O)C(O)CCC(=O)O] |
| 7,16,17-Trihydroxy DPAn- 6 (67) | 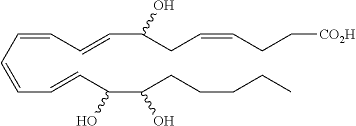 [SMILES: CCCCCC(O)C(O)/C=C/C=C\C=C/C=C/C(O)C/C=C\CCC(=O)O] |
| 8-Hydroxy DPAn-6 (68) | 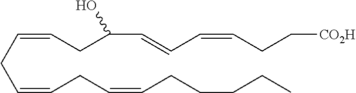 [SMILES: CCCCC/C=C\C/C=C\C/C=C\CC(O)/C=C/C=C\CCC(=O)O] |
| 14-Hydroxy DPAn-6 (69) | 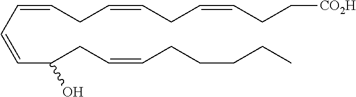 [SMILES: CCCCC/C=C\CC(O)/C=C\C=C/C/C=C\C/C=C\CCC(=O)O] |
| 13,17-Dihydroxy DPAn-6 (70) | 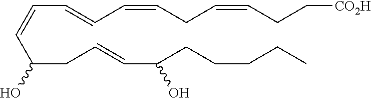 [SMILES: CCCCCC(O)/C=C/CC(O)\C=C/C=C/C=C\C/C=C\CCC(=O)O] |
| 7,14-Dihydroxy DPAn-6 (71) | 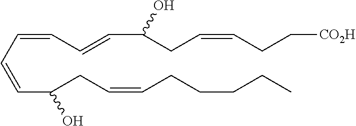 [SMILES: CCCCC/C=C\CC(O)\C=C/C=C\C=C\C(O)C/C=C\CCC(=O)O] |
| 8,14-Dihydroxy DPAn-6 (72) | 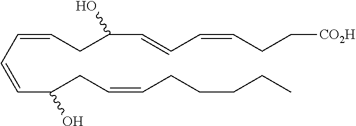 [SMILES: CCCCC/C=C\CC(O)/C=C\C=C/CC(O)/C=C/C=C\CCC(=O)O] |
| 11-Hydroxy DPAn-6 (73) | 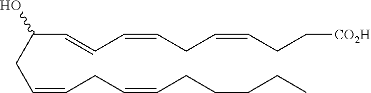 [SMILES: CCCCC/C=C\C/C=C\CC(O)/C=C/C=C\C/C=C\CCC(=O)O] |
| 10,17-Dihydroxy-DTAn-6 (74) | 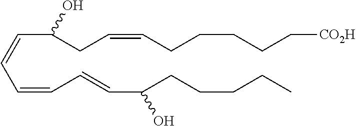 [SMILES: CCCCCC(O)/C=C/C=C\C=C/C(O)C/C=C\CCCCCC(=O)O] |
| 16,17-Dihydroxy-DTAn-6 (75) | 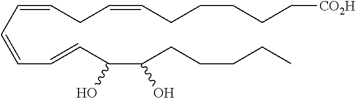 [SMILES: CCCCCC(O)C(O)/C=C/C=C\C=C/C/C=C\CCCCCC(=O)O] |
| 4,5-Dihydroxy-DTAn-6 (76) | 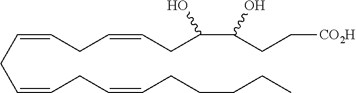 [SMILES: CCCCC/C=C\C/C=C\C/C=C\C/C=C\CC(O)C(O)CCC(=O)O] |
| 7,17-Dihydroxy-DTAn-6 (77) | 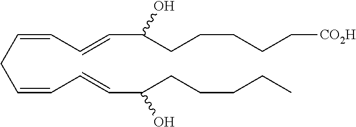 [SMILES: CCCCCC(O)/C=C/C=C\C/C=C\C=C\C(O)CCCCCC(=O)O] |
| 7-Hydroxy-DTAn-6 (78) | 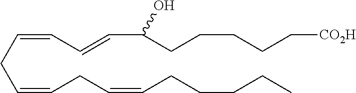 [SMILES: CCCCC/C=C\C/C=C\C/C=C\C=C\C(O)CCCCCC(=O)O] |
| 10-Hydroxy-DTAn-6 (79) | 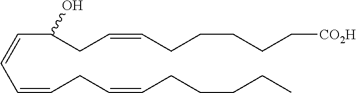 [SMILES: CCCCC/C=C\C/C=C\C=C/C(O)C/C=C\CCCCCC(=O)O] |
| 13-Hydroxy-DTAn-6 (80) | 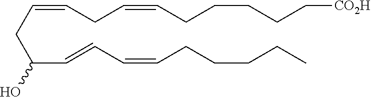 [SMILES: CCCCC/C=C\C=C\C(O)C/C=C\C/C=C\CCCCCC(=O)O] |
| 17-Hydroxy-DTAn-6 (81) | 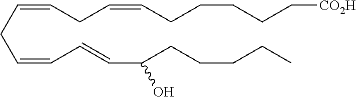 [SMILES: CCCCCC(O)/C=C/C=C\C/C=C\C/C=C\CCCCCC(=O)O] |
| 4,5,17-Trihydroxy-DTAn-6 (82) | 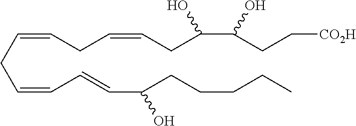 [SMILES: CCCCCC(O)/C=C/C=C\C/C=C\C/C=C\CC(O)C(O)CCC(=O)O] |
| 7,16,17-Trihydroxy-DTAn-6 (83) | 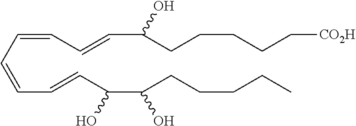 [SMILES: CCCCCC(O)C(O)/C=C/C=C\C=C/C=C/C(O)CCCCCC(=O)O] |
| 10,17-Dihydroxy DPAn-3 (84) | 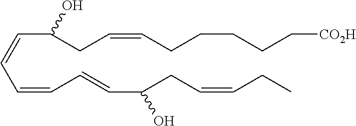 [SMILES: CC/C=C\CC(O)/C=C/C=C\C=C/C(O)C/C=C\CCCCCC(=O)O] |
| 10,20-Dihydroxy DPAn-3 (85) | 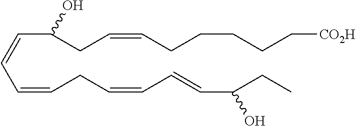 [SMILES: CCC(O)/C=C/C=C\C/C=C\C=C/C(O)C/C=C\CCCCCC(=O)O] |
| 13,20-Dihydroxy DPAn-3 (86) | 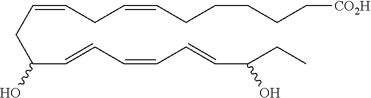 [SMILES: CCC(O)/C=C/C=C\C=C\C(O)C/C=C\C/C=C\CCCCCC(=O)O] |
| 16,17-Dihydroxy DPAn-3 (87) | 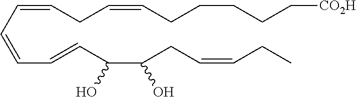 [SMILES: CC/C=C\CC(O)C(O)/C=C/C=C\C=C/C/C=C\CCCCCC(=O)O] |
| 7,17-Dihydroxy DPAn-3 (88) | 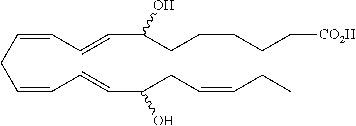 [SMILES: CC/C=C\CC(O)/C=C/C=C\C/C=C\C=C\C(O)CCCCCC(=O)O] |
| 7-Hydroxy DPAn-3 (89) | 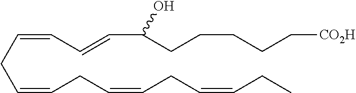 [SMILES: CC/C=C\C/C=C\C/C=C\C/C=C\C=C\C(O)CCCCCC(=O)O] |
| 10-Hydroxy DPAn-3 (90) | 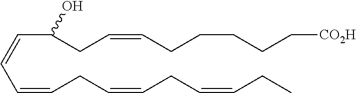 [SMILES: CC/C=C\C/C=C\C/C=C\C=C/C(O)C/C=C\CCCCCC(=O)O] |
| 13-Hydroxy DPAn-3 (91) | 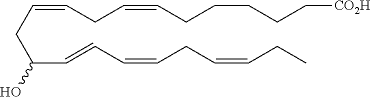 [SMILES: CC/C=C\C/C=C\C=C\C(O)C/C=C\C/C=C\CCCCCC(=O)O] |
| 17-Hydroxy DPAn-3 (92) | 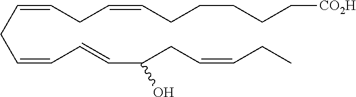 [SMILES: CC/C=C\CC(O)/C=C/C=C\C/C=C\C/C=C\CCCCCC(=O)O] |
| 7,16,17-Trihydroxy DPAn- 3 (93) | 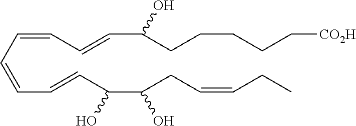 [SMILES: CC/C=C\CC(O)C(O)/C=C/C=C\C=C/C=C/C(O)CCCCCC(=O)O] |
| 16-Hydroxy DPAn-3 (94) | 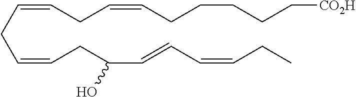 [SMILES: CC/C=C\C=C\C(O)C/C=C\C/C=C\C/C=C\CCCCCC(=O)O] |
| 11-Hydroxy DPAn-3 (95) | 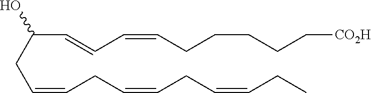 [SMILES: CC/C=C\C/C=C\C/C=C\CC(O)/C=C/C=C\CCCCCC(=O)O] |
| 14-Hydroxy DPAn-3 (96) | 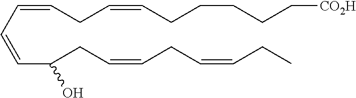 [SMILES: CC/C=C\C/C=C\CC(O)/C=C\C=C/C/C=C\CCCCCC(=O)O] |
| 8,14-Dihydroxy DPAn-3 (97) | 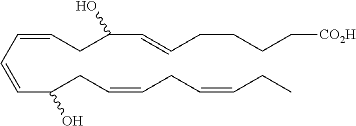 [SMILES: CC/C=C\C/C=C\CC(O)/C=C\C=C/CC(O)/C=C/CCCCC(=O)O] |
| 10,11-Epoxy DHA (98) | 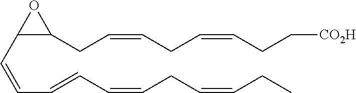 [SMILES: CC/C=C\C\C=C/C=C/C=C\C1OC1C/C=C\C/C=C\CCC(=O)O] |
| 13,14-Dihydroxy DHA (99) | 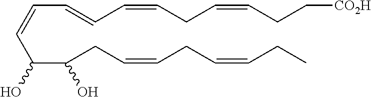 [SMILES: CC/C=C\C/C=C\CC(O)C(O)\C=C/C=C/C=C\C/C=C\CCC(=O)O] |
| 13,14-Epoxy DHA (100) | 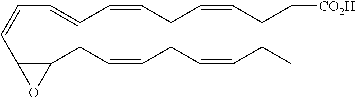 [SMILES: CC/C=C\C/C=C\CC1OC1\C=C/C=C/C=C\C/C=C\CCC(=O)O] |
| 19,20-Epoxy DHA (101) | 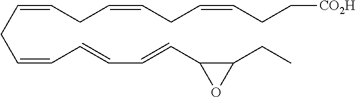 [SMILES: CCC1OC1/C=C/C=C/C=C\C/C=C\C/C=C\C/C=C\CCC(=O)O] |
| 7,8-Epoxy DHA (102) | 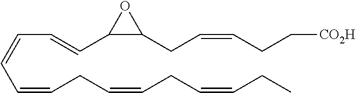 [SMILES: CC/C=C\C/C=C\C\C=C/C=C\C=C\C1OC1C/C=C\CCC(=O)O] |
| 4,5-Epoxy-17-OH DPA (103) | 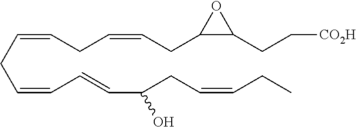 [SMILES: CC/C=C\CC(O)/C=C/C=C\C/C=C\C/C=C\CC1OC1CCC(=O)O] |
| 7,16,17-Trihydroxy DTAn- 3 (104) | 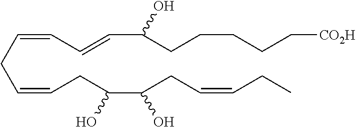 [SMILES: CC/C=C\CC(O)C(O)C/C=C\C/C=C\C=C\C(O)CCCCCC(=O)O] |
| 16,17-Dihydroxy DTAn-3 (105) | 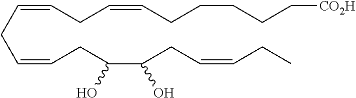 [SMILES: CC/C=C\CC(O)C(O)C/C=C\C/C=C\C/C=C\CCCCCC(=O)O] |
| 10,16,17-Trihydroxy DTRAn-6 (106) | 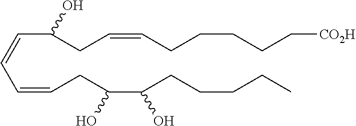 [SMILES: CCCCCC(O)C(O)C/C=C\C=C/C(O)C/C=C\CCCCCC(=O)O] |
| 16,17-Dihydroxy DTRAn-6 (107) | 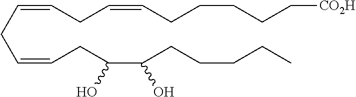 [SMILES: CCCCCC(O)C(O)C/C=C\C/C=C\C/C=C\CCCCCC(=O)O] |
| 7,16,17-Trihydroxy DTRAn-6 (108) | 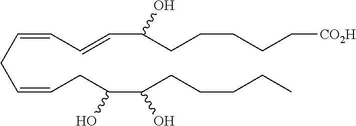 [SMILES: CCCCCC(O)C(O)C/C=C\C/C=C\C=C\C(O)CCCCCC(=O)O] |
| 15-epi-lipoxin A4 (109) | 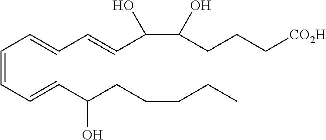 [SMILES: CCCCCC(O)/C=C/C=C\C=C\C=C\C(O)C(O)CCCC(=O)O] |
| 16,17-epoxy DHA (110) | 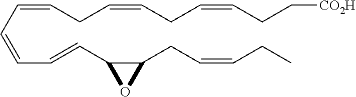 |
| 7,8-epoxy DPA (111) | 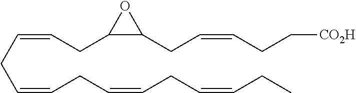 [SMILES: CC/C=C\C/C=C\C/C=C\C/C=C\CC1OC1C/C=C\CCC(=O)O] |
| 10,11 epoxy DPA (112) | 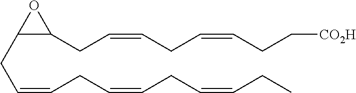 [SMILES: CC/C=C\C/C=C\C/C=C\CC1OC1C/C=C\C/C=C\CCC(=O)O] |
| 19,20 epoxy DPA (113) | 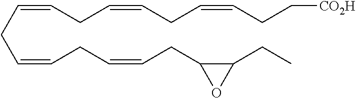 [SMILES: CCC1OC1C/C=C\C/C=C\C/C=C\C/C=C\C/C=C\CCC(=O)O] |
| 7-hydroxy DHA (114) | 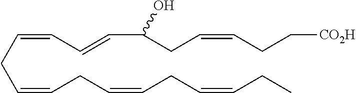 [SMILES: CC/C=C\C/C=C\C/C=C\C/C=C\C=C\C(O)C/C=C\CCC(=O)O] |
| 13,14 epoxy DPA (115) | 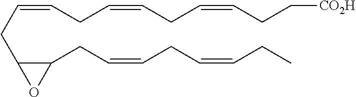 [SMILES: CC/C=C\C/C=C\CC1OC1C/C=C\C/C=C\C/C=C\CCC(=O)O] |
| 6-hydroxy GLA (116) | 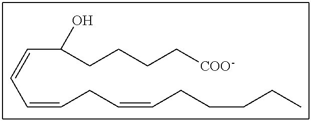 |
| 10-hydroxy GLA (117) | 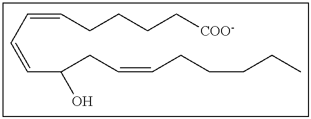 |
| 7-hydroxy GLA (118) | 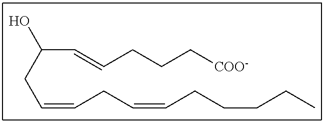 |
| 12-hydroxy GLA (119) | 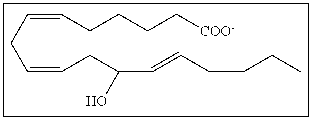 |
| 9-hydroxy GLA (120) | 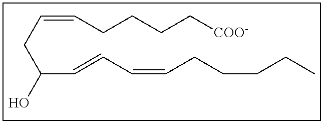 |
| 13-hydroxy GLA (121) | 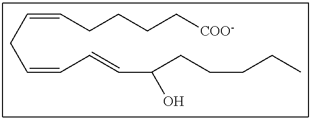 |
| 6,13 dihydroxy GLA (122) | 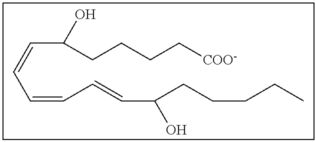 |
| 6-hydroxy SDA (123) | 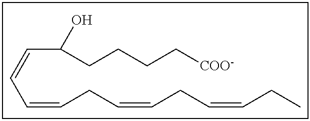 |
| 10-hydroxy SDA (124) | 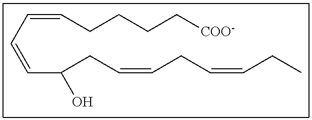 |
| 7-hydroxy SDA (125) | 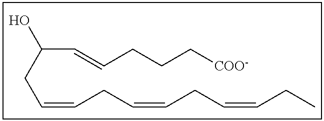 |
| 12-hydroxy SDA (126) | 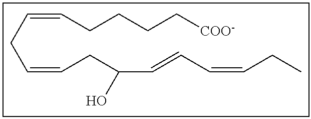 |
| 9-hydroxy SDA (127) | 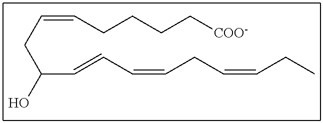 |
| 13-hydroxy SDA (128) | 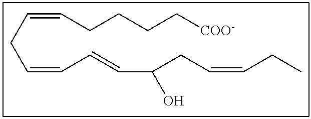 |
| 15-hydroxy SDA (129) | 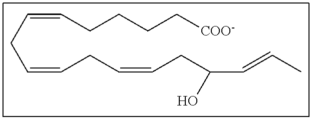 |
| 16-hydroxy SDA (130) | 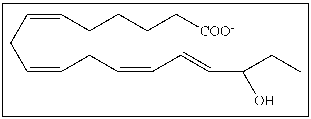 |
| 6,13 dihydroxy SDA (131) | 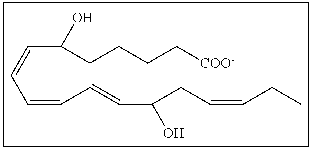 |
| 6,16 dihydroxy SDA (132) | 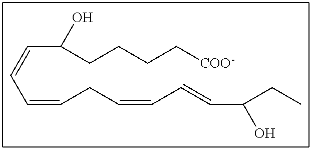 |

Other oxylipin compounds that are suitable for use in methods of the invention include analogs of the compounds shown in Table 1. Such compounds included but are not limited to those analogs wherein one or more double bonds are replaced by triple bonds, those wherein one or more carboxy groups are derivatized to form esters, amides or salts, those wherein the hydroxyl-bearing carbons are further derivatized (with, for example, a substituted or unsubstituted, branched or unbranched alkyl, alkenyl, or alkynyl group, substituted or unsubstituted aryl group, substituted or unsubstituted, branched or unbranched alkylaryl group, halogen atom) to form tertiary alcohols (or ethers, esters, or other derivatives thereof), those wherein one or more hydroxyl groups are derivatized to form esters or protected alcohols, or those having combinations of any of the foregoing modifications.

Further oxylipin compounds suitable for use in methods of the invention include the following: isolated docosanoids of docosapentaenoic acid (DPAn-6); monohydroxy, dihydroxy, and trihydroxy derivatives of DPAn-6; isolated docosanoids of docosapentaenoic acid (DPAn3); monohydroxy, dihydroxy, and trihydroxy derivatives of DPAn-3; isolated docosanoids of docosapentaenoic acid (DTAn-6); or monohydroxy, dihydroxy, and trihydroxy derivatives of DTAn-6.

Further compounds suitable for use in methods of the invention include compounds of formula I.

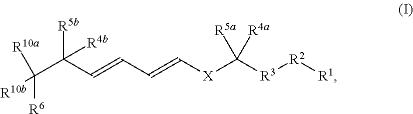

or a pharmaceutically acceptable salt thereof, wherein:

X is selected form —C≡C—, C(R)⁷)═C(R)⁷)—, -(cycloproply)-, -(cyclobutyl)-, -(cyclopentyl)-, and -(cyclohexyl)-;

R¹ is selected from —OR⁴, —N(R^{a})—SO₂R^{c} and —N(N^{a}))(R^{b}), wherein each of R^{a} and R^{b} is independently selected from H, C₁-C₆-alkyl, aryl, aralkyl, heteroaryl, and heteroaralkyl, and R¹ is selected from C₁-C₆-alkyl, aryl, aralkyl, heteroaryl, and heteroaralkyl;

R² is selected from —CH₂—, —C(O)—, —SO₂—, PO(OR)—, and tetrazole;

R is selected from hydrogen and alkyl;

R³ is selected from a carbocyclic ring, a heterocyclic ring, —(CH₂)ₙ—, CH₂C(O)CH₂2, and —CH₂—O—CH₂—, wherein:
n is an integer from 1 to 3;
any hydrogen atom an R³ is optionally and independently replaced by halo, (C₁-C₅)-alkyl, perfluoroalkyl, aryl, heteroaryl, hydroxy, or O—(C₁-C₅)-alkyl; and
any two hydrogen atoms bound to a common carbon atom in R³ are optionally taken together with the carbon atom to which they are bound to form a carbocyclic or heterocyclic ring;

each of R^{4a} and R^{4b} is independently selected from hydrogen, halo, —OH, —O—(C₁-C₅)-alkyl, —O-aryl, O-heteroaryl, —O—C(O)—(C₁-C₅)-alkyl, —C—C(O)-aryl, —O—C(O)-heteroaryl, —O—C(O)—(C₁-C₅)-alkyl, —O—C(O)—O-aryl, —O—C(O)—O-heteroaryl, and —OC(O)—N(R^{a})(R^{b}), wherein any alkyl, aryl or heteroaryl is optionally substituted with up to 3 substituents independently selected from halo, (C₁-C₅)-alkyl, O—(C₁-C₃)-alkyl, hydroxyl, carboxyl, ester, alkoxycarbonyl, acyl, thioester, thioacyl, thioether, amino, amido, acylamino, cyano, and nitro;

each of R^{5a} and R^{5b} is independently selected from hydrogen, halo, (C₁-C₅)-alkyl, perfluoroalkyl, aryl, and heteroaryl, preferably hydrogen, halo and (C₁-C₁)-alkyl;

R⁶ is selected from -phenyl, —(C₁-C₅)-alkyl, —(C₃-C₇)-cycloalkyl, —C≡CH-phenyl, —C≡C—(C₃-C₇)-cycloalkyl, —C≡C—(C₁-C₅)-alkyl, —C≡CH, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;

each R⁷ is independently selected from hydrogen and (C₁-C₅)-alkyl, or two occurrences of R⁷ may optionally be taken together with the carbons to which they are attached to form a 5- or 6-membered ring;

each of R^{10a} and R^{10b} is independently selected from hydrogen, (C₁-C₅)-alkyl, perfluoroalkyl, O—(C₁-C₅)-alkyl, aryl and heteroaryl, or R^{10a} and R^{10b} are taken together with the carbon atom to which they are bound to form a carbocyclic or heterocyclic ring;

and each double bond is independently in an E- or a Z-configuration.

In certain embodiments, R⁶ is —C≡CH when X is —C(R⁷)═C(R)⁷)— or -(cyclopropyl)-, or each of R^{4a} and R⁴ is hydrogen or halo, or each of R^{5a} and R^{5b} is halo, or R² is —CH₂.

In certain embodiments, R¹ is —OM, where M is a cation selected from ammonium, tetra-alkyl ammonium, Na, K, Mg, and Zn.

In certain embodiments, R² and R¹ together are

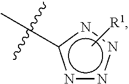

In certain embodiments X is —C≡C—. In certain embodiments, X is —C(R⁷)═C(R⁷)—, -(cyclopropyl)-, -(cyclobutyl)-, -(cyclopentyl)-, or -(cyclohexyl)-. In certain embodiments, X is —C(R)⁷)═C(R⁷)—. In certain embodiments, X is —C≡C—, -(cyclopropyl)-, -(cyclobutyl)-, -(cyclopentyl)-, or -(cyclohexyl)-. In certain embodiments, X is -(cyclopropyl)-. In certain embodiments, X is C≡C— or —C(R⁷)═C(R⁷)—. In certain embodiments wherein X is -(cyclopropyl)-, -(cyclobutyl)-, -(cyclopentyl)-, or -(cyclohexyl)-, the olefin and the carbon bearing R^{4a} are attached to adjacent carbons on the -(cyclopropyl)-, -(cyclobutyl)-, -(cylcopentyl)-, or -(cyclohexyl)- ring system.

In certain embodiments, R^{4b} is hydrogen. In certain embodiments, R^{4b} is halo, —OH, —O—(C₁-C₅)-alkyl, —O-aryl, O-heteroaryl, —O—C(O)—(C₁-C₅)-alkyl, —C—C(O)-aryl, —O—C(O)-aryl, —O—C(O)-heteroaryl, —C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro; In certain embodiments, R^{4b} is fluoro. In certain embodiments, R^{4b} is hydrogen, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—(C₁-C₅)-alkyl, —O—C(O)—O-aryl, —O—C(O)—O-heteroaryl, or —O—C—O—N(R^{a})(R^{b}), wherein any alkyl, aryl or heteroaryl is optionally substituted with up to 3 substituents independently selected from halo, (C₁-C₅)-alkyl, O—(C₁-C₃)-alkyl, hydroxyl, carboxyl, ester, alkoxycarbonyl, acyl, thioester, thioacyl, thioether, amino, amido, acylamino, cyano, and nitro; In certain embodiment, R^{ab} is selected from —OH, —O—(C₁-C₅)-alkyl, —O-aryl, O-heteroaryl, —O—C(O)—(C₁-C₃)-alkyl, —C—C(O)-aryl, —O—C(O)-heteroaryl, and —O—C(O)—N(R^{a})(R^{b}). In certain embodiments. R^{4b} is hydrogen, halo, —O—C(O)—O—(C₁-C₅)-alkyl, —O—C(O)—O-aryl, or —O—C(O)—O-heteroaryl, wherein any alkyl, aryl, or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from hydrogen, halo, —OH, or —O—(C₁-C₅)-alkyl. In certain embodiments, R^{4b} is —O—-aryl, O-heteroaryl, —(O)-C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —C—(O)—N(R^{a})(R^{b}), wherein any alkyl, aryl or heteroaryl is optionally substituted with up to 3 substituents independently selected from halo, (C₁-C₅)-alkyl, O—(C₁-C₃)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from —OH, or —O—(O)—(C₁-C₅)-alkyl. —O—-aryl, —O—-heteroaryl, —O—C(O)—(C₁-C₅)-alkyl, —O—C(O)—O-aryl, —O—C(O)—-heteroaryl, —O—C(O)—O—(C₁-C₅)-alkyl, —O—C(O)—O-aryl, —O—C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from hydrogen or halo.

In certain embodiments, R^{4b} is an (R) configuration. In certain embodiments, R^{4b} is in an (S) configuration.

In certain embodiments, R^{4a} is hydrogen. In certain embodiments, R^{4b} is halo, —OH, or —O—(C₁-C₅)-alkyl, —)-aryl, —O-heteroaryl, —(O)—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —C—(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is fluoro. In certain embodiments, R^{4a} is hydrogen, —OH, —O—(C₁-C₅)-alkyl, —O—-aryl, —O—-heteroaryl, —O—C(O)—(C₁-C₅)-alkyl, —O—C(O)—O-aryl, —O—C(O)—-heteroaryl, —O—C(O)—C—C(O)—O—(C₁-C₅)-alkyl, —O—C(O)—O-aryl, —O—C(O)—O-heteroaryl, or O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from —OH, —O—(C₁-C₅)-alkyl, —O-aryl, —O-heteroaryl, —(O)-C(O)—(C₁-C₅)-alkyl, —O—C(O)-aryl, —O—C(O)-heteroaryl, and —O—C(O)—N(R^{a})(R^{b}). In certain embodiments, R^{4a} is hydrogen, halo, —O—C(O)—O—(C₁-C₅)-alkyl, —O—C(O)—O-aryl, or —O—C(O)—O-heteroaryl, wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from hydrogen, halo, —OH, or —O—(C₁-C₅)-alkyl. In certain embodiments, R^{4a} is —O-aryl, —O-heteroaryl, —O—C(O)—(C₁-C₅)-alkyl, —O—C(O)—O-aryl, —O—C(O)—-heteroaryl, —O—C(O)—(C₁-C₅)-alkyl, —O—(O)-aryl, —C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from —OH, —O—(C₁-C₅)-alkyl, —O-aryl, —O-heteroaryl, —O—C(O)—(C₁-C₅)-alkyl, —O—C(O)-aryl, —O—C(O)-heteroaryl, —O—C(O)—(C₁-C₅)-alkyl, —O—(O)—O-aryl, —C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from hydrogen or halo.

In certain embodiments, R^{4a} is in an (S) configuration. In certain embodiments, R^{4a} is in an (R) configuration.

In certain embodiments wherein R^{4a} is —OH, R^{5a} is selected from hydrogen or (C₁-C₅)-alkyl. In certain embodiments wherein R^{4a} is selected from —OH, —(O)-C—(C₁-C₅)-alkyl, —O—C(O)-aryl, —O-heteroaryl, —O—C(O)—(C₁-C₅)-alkyl, —O—C(O)-aryl, —O—C(O)-heteroaryl, —O—C(O)—O—(C₁-C₅)-alkyl, O—O—C(O)—O—aryl, —O—C(O)—-heteroaryl, —O—C(O)—C—C(O)—O—(C₁-C₅)-alkyl, —O—C(O)—O-aryl, —O—C(O)—O-heteroaryl, and —O—C(O)—N(R^{a})(R_{b}), R^{5a} is selected from hydrogen or (C₁-C₅)-alkyl. In certain embodiments, R^{5a} is fluoro. In certain embodiment, R^{5a} is selected from hydrogen and (C₁-C₅)-alkyl.

In certain embodiments wherein R^{4b} is —OH, R^{5b} is selected from hydrogen or (C₁-C₅)-alkyl. In certain embodiments wherein R^{4b} is selected from —OH, —(O)-C—(C₁-C₅)-alkyl, —O—C(O)-aryl, —O-heteroaryl, —O—C(O)—(C₁-C₅)-alkyl, —O—C(O)-aryl, —O—C(O)-heteroaryl, —O—C(O)—O—(C₁-C₅)-alkyl, O—O—C(O)—O—aryl, —O—C(O)—-heteroaryl, and —O—C(O)—N(R^{a})(R_{b}), R^{5b} is selected from hydrogen or (C₁-C₅)-alkyl. In certain embodiments, R^{5b} is fluoro. In certain embodiment, R^{5b} is selected from hydrogen and (C₁-C₅)-alkyl.

In certain, R² is —CH₂—. In certain embodiment, R² is —C(O)—.

In certain embodiment, R^{a} is selected from H and C₁-C₆-alkyl. In certain embodiment, R^{a} is selected from aryl, aralkyl, heteroaryl, and heteroaralkyl.

In certain embodiment, R^{b} is selected from H and C₁-C₆-alkyl. In certain embodiment, R^{b} is selected from aryl, aralkyl, heteroaryl, and heteroaralkyl.

In certain embodiments, R^{c} is C₁-C₆-alkyl, aryl, or heteroaryl. In certain embodiment, R^{c} is selected from aryl, aralkyl, heteroaryl, and heteroaralkyl.

In certain embodiments wherein R³ is selected from a carbocyclic ring, a heterocyclic ring, —(CH₂)ₐ—, and CH₂C(O)CH₂, any hydrogen atom in R⁴ is optionally and independently replace by halo, (C₁-C₅)-alkyl, perfluoroalkyl, aryl, heteroaryl, hydroxy, or O—(C₁-C₅)-alkyl. In certain embodiment where R₃ is —CH₂O—OH₂, any hydrogen atom in R³ is optionally and independently replaced by halo, (C₁-CH₅)-alkyl, perfluoroalkyl, aryl, heteroaryl, or O—(C₁-C₅)-alkyl. In certain embodiments, R³ is selected from —(CH₂)_{A}, and —CH₂—O—CH₂, wherein n is an integer from 1 to 3, and up to two hydrogen atoms in R³ are optionally and independently replaced by (C₁-C₅)-alkyl. In certain embodiments, R³ is selected from a carbocyclic ring, a heterocyclic ring, and CH₂C(O)CH₂, wherein n is an integer from 1 to 3; any hydrogen atom in R³ optionally and independently replaced by halo, (CH₁-CH₅)-alkyl, perfluoroalkyl, aryl, heteroaryl, hydroxy, or O—(C₁-C₅)-alkyl; and any two hydrogen atoms bound to a common carbon atom in R³ are optionally taken together with the carbon atom to which they are bound to form a carbocyclic or heterocyclic ring.

In certain embodiments, R^{16a} is hydrogen. In certain embodiments, R^{10a} is selected from (C₁-C₅)-alkyl, perfluoroalkyl, O—(C₁-C₅)-alkyl, and heteroaryl, or R^{10a} is taken together with R^{10b} and the carbon atom to which they are bound to form a carbocyclic or heterocyclic ring.

In certain embodiments, R^{16b} is hydrogen. In certain embodiments, R^{10b} is selected from (C₁-C₅)-alkyl, perfluoroalkyl, O—(C₁-C₅)-alkyl, aryl and heteroaryl, or R^{10b} is taken together with R^{10a} and the carbon atom to which they are bound to form a carbocyclic or heterocyclic ring.

In certain embodiment, R¹ is —OR^{a}. In certain embodiment, R¹ is selected from —N(R^{a})—SO₂—R^{c} and —N(R^{a})(R^{b}). In certain embodiment, R¹ is —N(R^{a})—SO₂R^{a}. In certain embodiments, R¹ is selected from —OR^{a} and —N(R^{a})(R^{b}). In certain embodiments, R¹ is —N(R^{a})(R^{b}). In certain embodiment, R¹is selected from —OR², and —N(R^{a})—SO₂—R^{a}.

In certain embodiments, R⁷ is hydrogen. In certain embodiments, R⁷ is (C₁-C₅)-alkyl or two occurrences of R⁷ may optionally be taken together with the carbons to which they are attached to form a 5- or 6-membered ring.

In certain embodiment, X is —C≡C— and R^{4b} is hydrogen.

In certain embodiment, X is —C≡C— and R^{4a} is hydrogen.

In certain embodiment, X is —C≡C— and R^{4a} is fluoro, and R^{5a} is fluoro.

In certain embodiment, X is —C≡C— and R^{4b} is fluoro, and R^{5b} is fluoro.

In certain embodiment, X is —C≡C— and each of R^{4a} and R^{4b} is independently. selected from —OH, —(O)—C—(C₁-C₅)-alkyl, —O-aryl, —O-heteroaryl, —O—C(O)—(C₁-C₅)-alkyl, —O—C(O)-aryl, —O—C(O)-heteroaryl, and —O—C(O)—N(R^{a})(R_{b}).

In certain embodiment, X is -(cyclopropyl)-, -(cyclobutyl)-, -(cyclopentyl)-, and -(cyclohexyl)-. In certain embodiments, X is -(cyclopropyl)-.

In certain embodiments, X is —C(R⁷)═C(R⁷).

In certain embodiment, R¹ is R¹ and R^{b} is independently selected from H and (C₁-C₆)-alkyl; R^{c} is (C₁-C₆)-alkyl; R³ is selected from (13 (CH₂)ₐ— and —CH₂O—CH₂, wherein n is an integer from 1 to 3, and up to two hydrogen atoms in R^{4a} and R^{4b} is independently selected from hydrogen, halo, —OH, —O—(C₁-C₅)-alkyl; and each of R^{10a} and R^{16b} is hydrogen.

In certain embodiments, each double bond is in an E-configuration. In certain embodiments, each double bond is in a Z-configuration. In certain embodiments, one double bond is in an E-configuration and one double bond is in a Z-configuration.

In certain embodiments, the invention contemplates any combination of the foregoing. Those skilled in the art will recognize that all specific combinations of the individual possible residue of the variable regions of the compounds as disclosed herein, e.g., R¹, R², R³, R^{4a}, R^{4b}, R^{5a}, R^{5b}, R⁶, R⁷, R^{10a}, R^{10b}, R^{a}, R^{b}, R^{c}, n and X, are within the scope of the invention. As an example, any of the various particular recited embodiments for R^{4a} may be combined with any of the various particular recited embodiments of X.

In certain embodiments, the compound is selected from any one of:

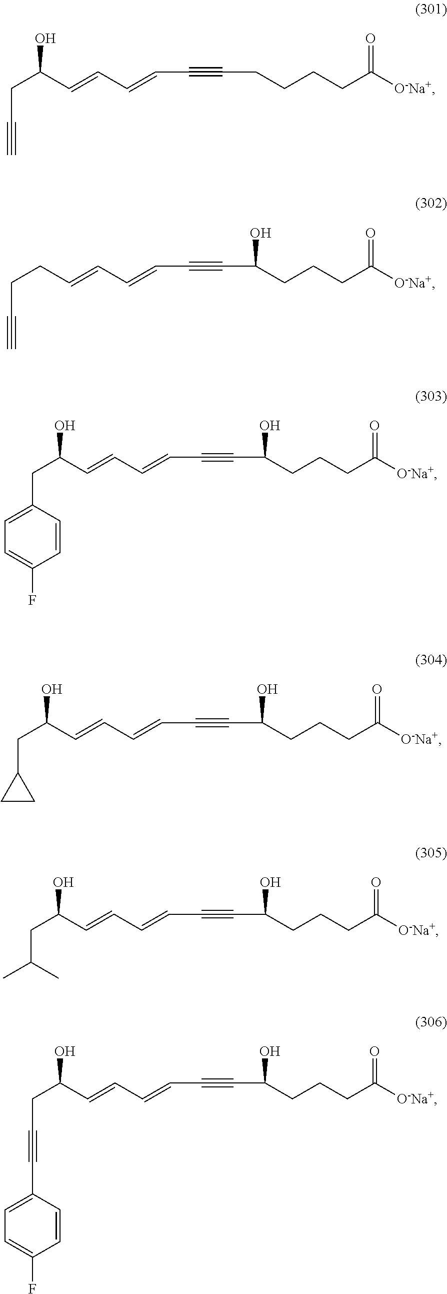

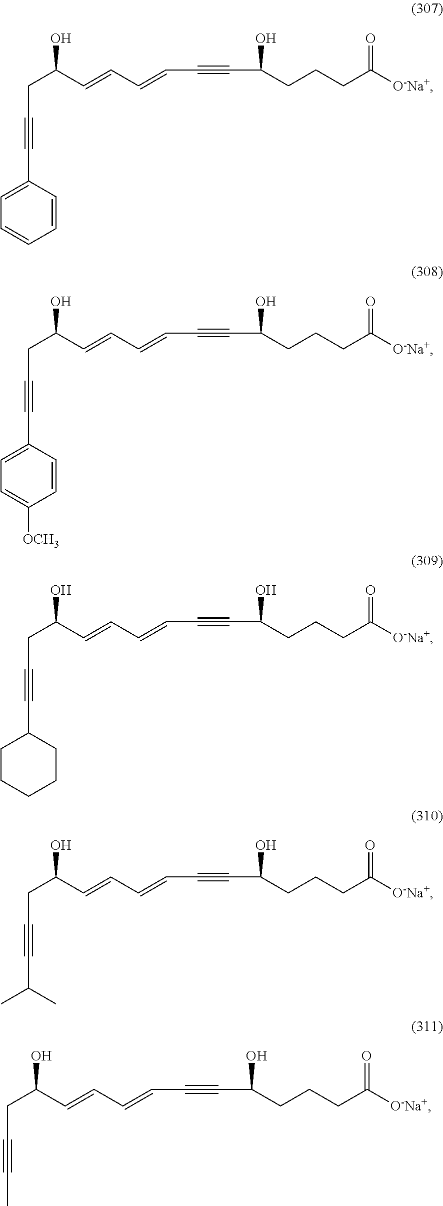

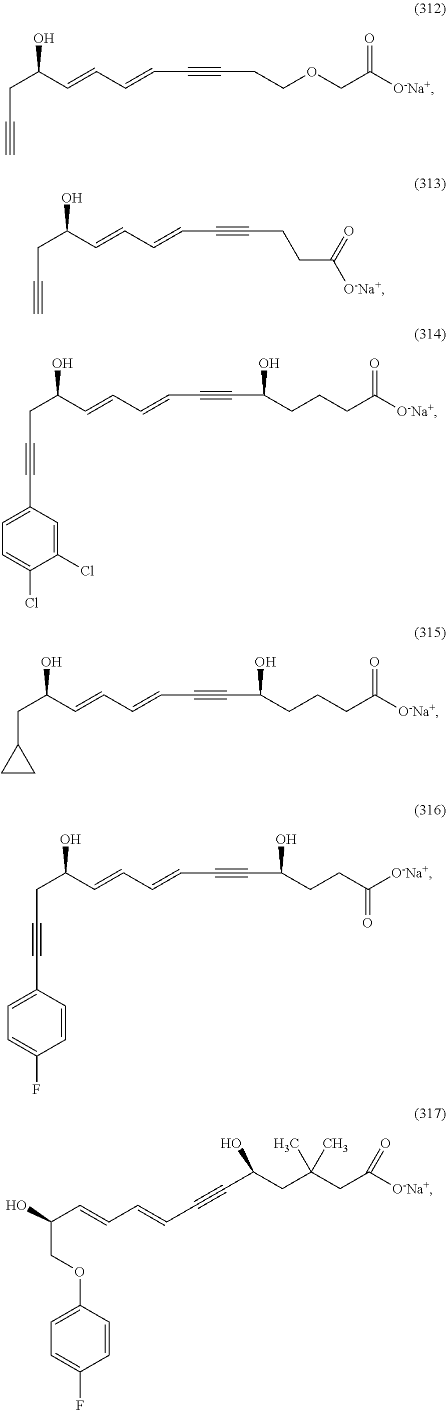

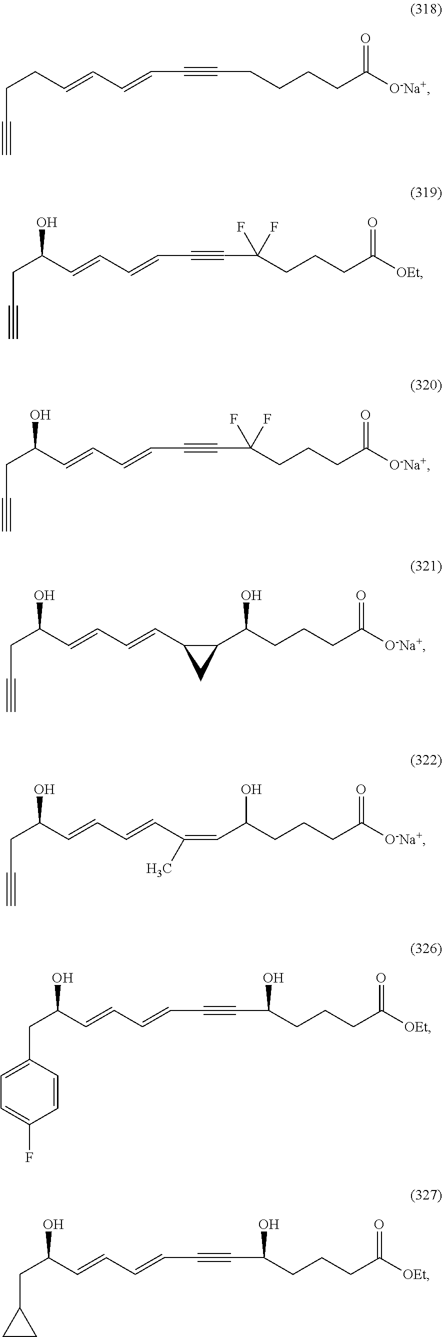

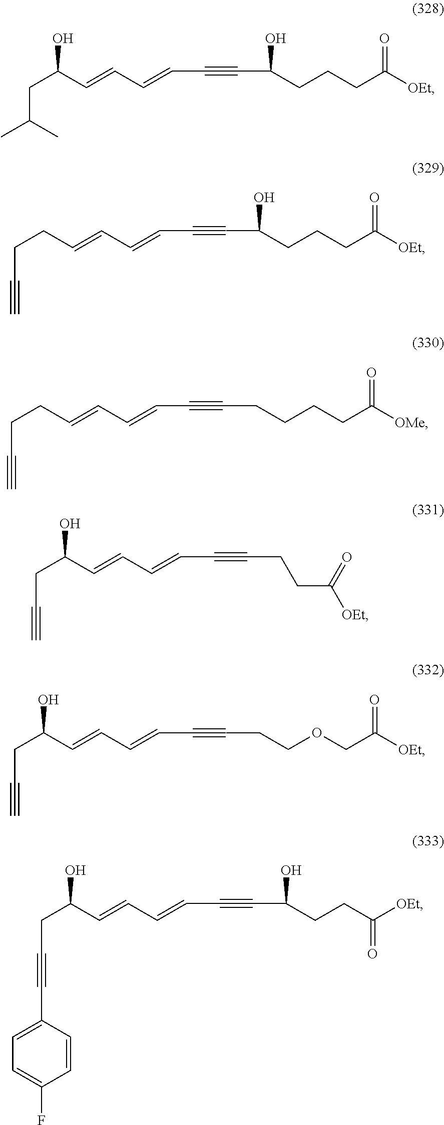 [SMILES: CCOC(=O)CCC[C@H](O)C#C/C=C/C=C/[C@H](O)CC(C)C ; C#CCC/C=C/C=C/C#C[C@@H](O)CCCC(=O)OCC ; C#CCC/C=C/C=C/C#CCCCCC(=O)OC ; C#CC[C@@H](O)/C=C/C=C/C#CCCC(=O)OCC ; C#CC[C@@H](O)/C=C/C=C/C#CCCOCC(=O)OCC ; CCOC(=O)CC[C@H](O)C#C/C=C/C=C/[C@H](O)CC#Cc1ccc(F)cc1]

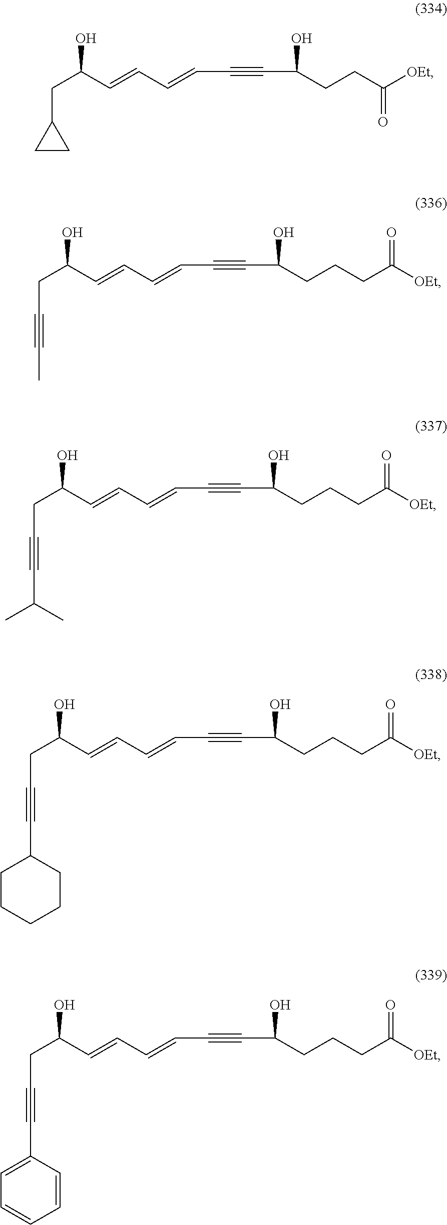 [SMILES: CCOC(=O)CC[C@H](O)C#C/C=C/C=C/[C@H](O)CC1CC1 ; CC#CC[C@@H](O)/C=C/C=C/C#C[C@@H](O)CCCC(=O)OCC ; CCOC(=O)CCC[C@H](O)C#C/C=C/C=C/[C@H](O)CC#CC(C)C ; CCOC(=O)CCC[C@H](O)C#C/C=C/C=C/[C@H](O)CC#CC1CCCCC1 ; CCOC(=O)CCC[C@H](O)C#C/C=C/C=C/[C@H](O)CC#Cc1ccccc1]

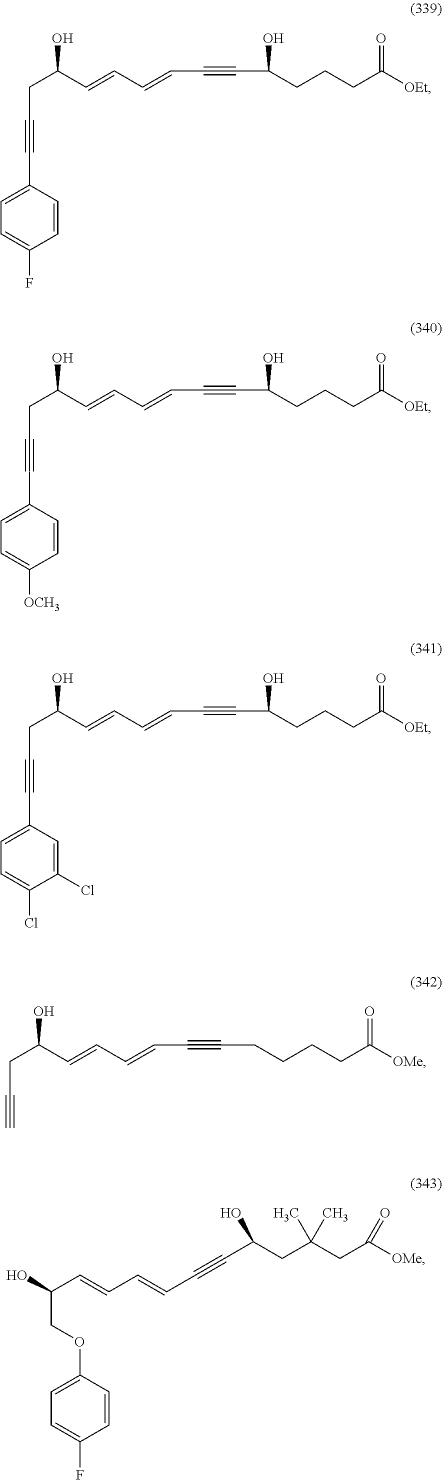 [SMILES: CCOC(=O)CCC[C@H](O)C#C/C=C/C=C/[C@H](O)CC#Cc1ccc(F)cc1 ; CCOC(=O)CCC[C@H](O)C#C/C=C/C=C/[C@H](O)CC#Cc1ccc(OC)cc1 ; CCOC(=O)CCC[C@H](O)C#C/C=C/C=C/[C@H](O)CC#Cc1ccc(Cl)c(Cl)c1 ; C#CC[C@@H](O)/C=C/C=C/C#CCCCCC(=O)OC ; COC(=O)CC(C)(C)C[C@H](O)C#C/C=C/C=C/[C@H](O)COc1ccc(F)cc1]

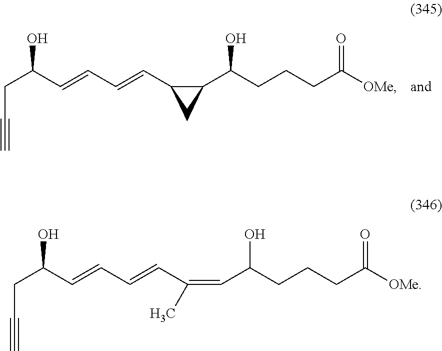 [SMILES: C#CC[C@@H](O)/C=C/C=C/[C@@H]1C[C@@H]1[C@@H](O)CCCC(=O)OC ; C#CC[C@@H](O)/C=C/C=C/C(C)=C\C(O)CCCC(=O)OC]

Further compounds suitable for use in methods of the invention include compounds of the formula II,

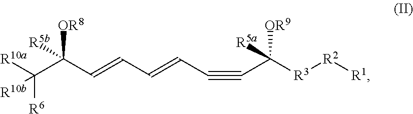

or formula III,

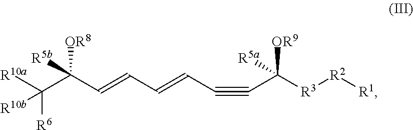

or a pharmaceutically acceptable
salt of either of the foregoing, wherein:

R¹ is selected from —OR^{a}, —N(R^{a})—SO₂R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;

R² is selected from —(C(O)—, SO₂—, PO(OR)—, and tetrazole;

R is selected from hydrogen and alkyl;

R³ is selected from —(CH₂)ₐ— and —CH₂—O—CH₂, wherein n is an integer from 1 to 3; and optionally up to two hydrogen atoms in R^{b} are independently replaced by halo, (C₁-C₅)-alkyl, or O—(C₁-C₅)-alkyl;

each of R^{5a} and R^{5b} is independently selected from hydrogen, (C₁-C₅)-alkyl, perfluoroalkyl, aryl, and heteroaryl, preferably hydrogen and (C₁-C₅)-alkyl;

R^{b} is selected from —C≡CH, -phenyl, —(C₁-C₅)-alkyl, —(C₃-C₇)-cycloalkyl, —C≡C-phenyl, —C≡C—(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester thioacyl, thioester, amino, amido, acylamino, cyano and nitro;

each of R⁸ and R⁹ are independently selected from hydrogen, —(C₁-C₅)-alkyl, -aryl, -heteroaryl, —C(O)—(C₁-C₅)-alkyl, —C(O)-aryl, —C(O)-heteroaryl, —C(O)—O—(C₁-C₅)-alkyl, —C(O)—-aryl, —C(O)—O-heteroaryl, and —C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester thioacyl, thioether, amino, amido, acylamino, cyano and nitro;

each of R^{10a} and R^{10b} is independently selected from hydrogen, (C₁-C₅)-alkyl, perfluoroalkyl, O—(C₁-C₅)-alkyl, aryl and heteroaryl, or

R^{10a} and R^{10b} are taken together with the carbon atom to which they are bound to form a carbocyclic or heterocyclic ring; and

wherein each double bond is independently in an E- or a Z-configuration.

In certain embodiment, R¹ is —OM, where M is a cation selected from ammonium, tetra-alkyl ammonium, Na, K, Mg, and Zn.

In certain embodiment, R² and R¹ together are

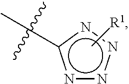

In certain embodiments, R² is —C(O)—. In certain embodiment, R¹ —OR^{a}, wherein R^{a} is hydrogen or C₁-C₅-alkyl. In certain embodiments, R² is —(CH₂)ₐ—, wherein n is 3. In certain embodiments, R⁶ is —C≡CH. In certain embodiments, R^{5a} is hydrogen. In certain embodiments, R^{5b} is hydrogen. In certain embodiments, R^{10a} is hydrogen. In certain embodiments, R^{10b} is hydrogen. In certain embodiments, R² is —C(O)—, R¹ is —OR^{a}, wherein R^{a} is C₁-C₆-alkyl, R³ is —(CH₂)ₐ—, wherein n is 3, R⁶ is —C≡CH, R^{5a} is hydrogen, R^{5b} is hydrogen, R^{10a} is hydrogen, and R^{10b} is hydrogen.

In certain embodiments, the compound is selected from any one of:

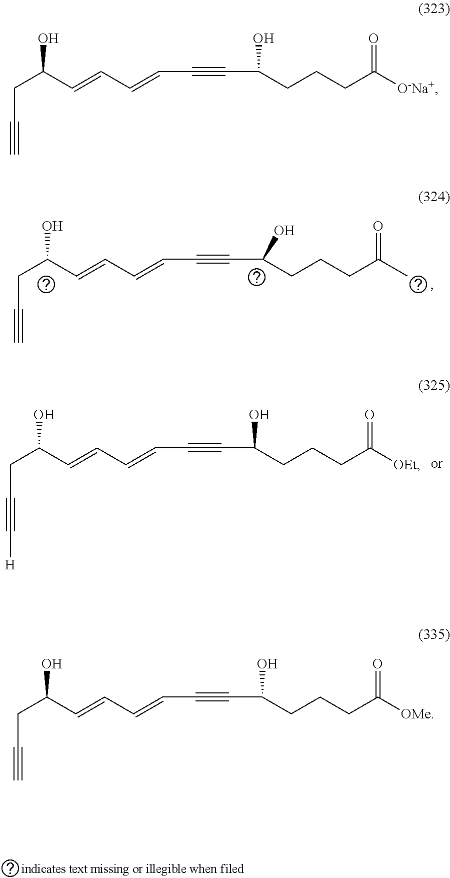

In certain embodiments, the invention contemplates any combination of the foregoing. Those skilled in the art will recognize that all specific combinations of the individual possible residues of the variable regions of the compounds as disclosed herein, e.g., R¹, R², R³, R^{5a}, R^{5b}, R⁶, R⁸, R⁹, R^{10a}, R^{10b}, R^{a}, R^{b}, R^{c}, and n, are within the scope of the invention. As am example, any of the various particular recited embodiments for R⁸ may be combined with any of the various particular recited embodiments of R⁶.

These term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarylC(NH)—.

The term “acyloxy” is art-recognized and refers to a group represented by the general formula hydrocarbylC(O)O—, preferably alkylC(C)O—.

The term “alkoxy” refers to an alkyl group, preferably a lower alkyl group, having an oxygen attached thereto. Representative alkoxy groups included methoxy, ethoxy, propoxy, tert-butoxy and the like.

The “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents, include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₂-C₃₆ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replaced a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), and alkoxyl, a phosphoryl, a phosphate, a phosphonate, a phosphinate, an amino, an amido, an amidine, an imine, a cyano, a nitro, an azido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted an unsubstituted forms of amino, azido, amino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branch-chain alkyl groups that contain from x to y carbons n the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to included both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

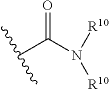

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

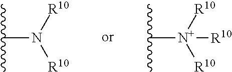

wherein each R¹⁰ independently represented a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

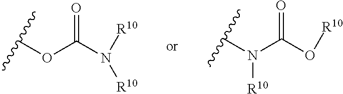

wherein each R¹⁶ independently represent hydrogen or a hydrocarbyl group, or both R¹⁰ groups taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclyalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be ether symmetrical or unsymmetrical. Examples of ethers include, but are not limited, to heterocycle-O— heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5 - to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cyloalkenyls, cycloalkynyls, aryls, heteroaryls, and/or heterocyclyls. heteroaryl groups include, for example, pyrrole, furan, thiophene, imidazole, oxazole, thiozole, pyrazole, pyridine, pyrazine, pyridazine, and pyrimidine, and the like.

The term “heteroatom” as used herein means as atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteratoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycoalkynyls, aryls, heteroaryls, and/or heterocyclyls. Heterocyclyl groups include, for example, piperidine, piperazine, pyrrolidine, morpholine, lactones, lactums, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably is six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituents, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate , a phosphinate, a amino, an amido, an amidine, a imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

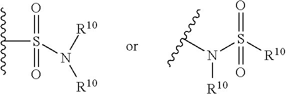

wherein each R¹⁰ independently represents hydrogen or hydrocarbyl, or both R¹⁰ groups taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

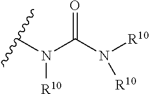

wherein each R¹⁰ independently represent hydrogen or a hydrocarbyl, or two occurrences of R¹⁰ taken together with the intervening atom(s) complete a heterocycle having from 4 to 8 atoms in the ring structure.

The term “prodrug” is intended to encompass compounds which, under physiologic conditions, are converted into the therapeutically active agents of the present invention (e.g., a compound of formula A or formulae 1-49 or I-III, a lipoxin compound, or an oxylipin compound). A common method for making a prodrug is to include one or more selected moieties which are hydrolyzed under physiologic conditions to reveal the desired molecule. In other embodiments, the prodrug is converted by an enzymatic activity of the host animal. For example, esters (e.g., esters of alcohols or carboxylic acids) are preferred prodrugs of the present invention. In certain embodiments, some or all of the compounds of formula A, compounds of any one of formulae 1-49 or I-III, lipoxins, or oxylipins, all or a portion of a Compound of formula A, compound of any one of formulae 1-49 or I-III, lipoxin, or oxylipin in a formulation represented above can be replaced with the corresponding suitable prodrug, e.g., wherein a hydroxyl or carboxyl acid present in the parent compound is presented as an ester.

“Protecting group” refers to a group of atoms that, when attached to a reactive functional group in a molecule, mask, reduce or prevent the reactivity of the functional group. Typically, a protecting group may he selectively removed as desired during the course of a synthesis. Examples of protecting groups can be found in Greene and Wuts, Protective Groups in Organic Chemistry, 3^{rd} Ed., 1999, John Wiley & Sons, N.Y. and Harrison et al., Compendium of Synthetic Organic Methods, Vols. 1-8, 1971-1996, John Wiley & Sons, N.Y. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers. p The term “treating” refers to: preventing a disease, disorder or condition from occurring in a cell, a tissue, a system, animal or human which may be predisposed to the disease, disorder and/or condition but has a not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays, the onset or reduces the severity of one or more symptoms of the disorder or condition relative to the untreated control sample.

As used herein, a “complex disorder having an inflammatory component” is a disease where the initial pathology/dysfunction in a particular tissue or organ that is vital for the systems biology function of an individual will secondarily lead to systemic metabolic derangement and/or tissue stress causing, or further enhancing, activation of the immune system leading to dysfunction in several organs vital for body homeostatis.

The synthesis of each of the PPAR, LXR, RXR, or HNF-4 agonists, or sirtuin-activating compounds and each of the compounds of formula A, compounds of any one of formulae 1-49 or I-III, lipoxins, or oxylipins set forth above can be achieved by methods well-known in the art. For example, the synthesis of compounds of formula A or formulae 1-49 is set forth in US 2003/0191184, WO2004/014835, WO 2004/078143, U.S. Pat. No. 6,670,396, US 2003/0236423 and US 2005/0228047, all of which are herein incorporated by reference. The synthesis of lipoxin compounds is set forth in US 2002/0107289, US 2004/0019110, US 2006/0009521, US 2005/0203184, US 2005/103443, all of which are herein incorporated by reference. The preparation of oxylipin compounds is set forth in WO2006/055965, WO2007/090162, and WO 2008/103753, all of which are herein incorporated by reference. The preparation of sirtuin-activating compounds is set forth in the following applications: WO2007019416, WO2007008548, WO2006105440, WO2006127987, WO2006105403, WO2006094237, WO2006094236, WO2006094235, WO2006076681, WO2006079021, US2007043050, US2007037809, US2007037827, US2006276393, WO200609248, WO2006078941, WO2005069998, WO2006096780, WO2007104867, US2007212395, WO2006138418, US2006292099, JP2006298876, and US2006025337, all of which are herein incorporated by reference. The synthesis of compounds of formulae I-III is disclosed in U.S. Provisional Patent Application No. 61/194,093, file on Sep. 23, 2008, entitled “Therapeutic Compounds,” to Schwartz.

The certain embodiments, the patient to be treated by a method of the invention may already be receiving an anti-inflammatory drug (other than a PPAR, LXR, RXR, or HNF-4 agonist). In one preferred embodiment, the patient is already taking a PPAR agonist (e.g., a PPARα, PPARβ/δ, or a PPARγ agonist), an LXR agonist (e.g., an LXRα or LXRβ agonist), an RXR agonist (e.g., an RXRα, RXRβ, or an RXRγ agonist), an HNF-4 agonist, or a sirtuin-activating compound such as one of the PPAR, LXR, RXR, or HNF-4 agonists, or sirtuin-activating compounds described above, and will continue to take that drug conjointly with a compounds of formula A, a compound of any one of formulae 1-49 or I-III, a lipoxin compound, an oxylipin compound, or a combination of aspirin and an omega-3 fatty acid.

In a related embodiment, the invention provides a method of reducing the dose of a PPAR, LXR, RXR, or HNF-4 agonist, or a sirtuin-activating compound required to achieve a desired anti-inflammatory properties is highly desirable due to side effects associated with certain PPAR agonists. Side effects of PPAR agonists, such as thiazolidinediones, include weight gain, edema, fluid retention that may aggravate heart failure, and, in some cases, liver toxicity.

In this embodiment, the dose of a PPAR agonist (e.g., a PPARα, PPARβ/δ, or a PPARγ agonist), an LXR agonist (e.g., an LXRα or LXRβ agonist), an RXR agonist (e.g., an RXRα, RXRβ, or an RXRγ agonist), an HNF-4 agonist, or a sirtuin-activating compound is reduced by at least 5%, at least 10%, at least 15%, at least 20%, at least 25%, at least 30%, at least 40%, at least 50%, at least 60%, at least 70%, at least 80%, at least 90%, or more relative to a dose in the absence of conjoint administration. The actual reduction in PPAR agonist (e.g., a PPARα, PPARβ/δ, or PPARγ agonist), LXR agonist (e.g., an LXRα or LXRβ agonist), RXR agonist (e.g., RXRα, RXRβ, or an RXRγ agonist), HNF-4 agonist dose, or sirtuin-activating compound will depend upon the nature and amount of the compound of formula A, compound of any one of formulae 1-49 or I-III, lipoxin, oxylipin, or combination of aspirin and an omega-3 fatty acid being administered, the reduction in inflammation desired, and other factors set forth elsewhere in this application that are typically considered in treating a disease or condition. The amount of the compound of formula A, compound of any one of formulae 1-49 or I-III, lipoxin, oxylipin, or combination of aspirin and an omega-3 fatty acid administered in this method will also depend upon the factors set forth above, as well as the nature and amount of PPAR agonist (e.g., a PPARα, PPARβ/δ, or a PPARγ agonist), LXR agonist (e.g., LXRα or LXRβ agonist), RXR agonist (e.g., an RXRα, RXRβ, or an RXRγ agonist), HNF-4 agonist, or a sirtuin-activating compound being administered. In certain embodiments, the amount of compound of formula A, compound of any one of formulae 1-49 or I-III, lipoxin, oxylipin, or combination of aspirin and an omega-3 fatty acid administered in this method is less than 5%, less than 10%, less than 15%, less than 20%, less than 25%, less than 30%, less than 40%, less than 50%, less than 60%, lest than 70%, less than 80%, or lest than 90% of the dose of compound of Formula A, compound of any one of formulae 1-49 or I-III, lipoxin, oxylipin, or combination of aspirin and an omega-3 fatty acid required to produce an anti-inflammatory effect without conjoint administration with a PPAR agonist (e.g., a PPARα, PPARβ/δ, or a PPARγ agonist), LXR agonist (e.g., LXRα or LXRβ agonist), RXR agonist (e.g., an RXRα, RXRβ, or an RXRγ agonist), HNF-4 agonist, or a sirtuin-activating compound.

In yet another embodiment, the invention provides a composition comprising a PPAR agonist (e.g., a PPARα, PPARβ/δ, or a PPARγ agonist), LXR agonist (e.g., LXRα or LXRβ agonist), an RXR agonist (e.g., an RXRα, RXRβ, or an RXRγ agonist), an HNF-4 agonist, or a sirtuin-activating compound and a compound of formula A, compound of any one of formulae 1-49 or I-III, lipoxin, oxylipin, or combination of aspirin and an omega-3 fatty acid, and a pharmaceutically acceptable carrier. In these compositions, PPAR agonist (e.g., a PPARα, PPARβ/δ, or a PPARγ agonist), LXR agonist (e.g., LXRα or LXRβ agonist), RXR agonist (e.g., an RXRα, RXRβ, or an RXRγ agonist), HNF-4 agonist, or a sirtuin-activating compound may be selected from any suitable PPAR, LXR, RXR, or HNF-4 agonist, or sirtuin-activating compound. In certain embodiments, the PPAR, LXR, RXR, or HNF-4 agonist, or sirtuin-activating compound is one of the PPAR, LXR, RXR, or HNF-4 agonists, or sirtuin-activating compounds set forth above. Similarly, the compound of formula A or of any of formulae 1-49 or I-III may be selected from any such compound known in the art, such one of the compounds set forth above. Similarly, the lipoxin may be selected from any suitable lipoxin. In certain embodiments, the lipoxin is one of the lipoxins set forth above. Similarly, the oxylipin may be selected from any suitable oxylipin. In certain embodiments, the oxylipin is one of the oxylipins set forth above. The amount of PPAR agonist (e.g., a PPARα, PPARβ/δ, or a PPARγ agonist), LXR agonist (e.g., LXRα or LXRβ agonist), RXR agonist (e.g., an RXRα, RXRβ, RXRγ agonist), HNF-4 agonist, or a sirtuin-activating compound in this combination composition is less than 5%, less than 10%, less than 15%, less than 20%, less than 25%, less than 30%, less than 40%, less than 50%, less than 60%, less than 70%, less than 80%, less than 90%, or less than 100% of the amount of PPAR agonist (e.g., PPARα, PPAR β/δ, or PPARγ agonist), LXR agonist (e.g., LXRα or LXRβ agonist), RXR agonist (e.g., RXRα, RXRβ, or RXRγ agonist), HNF-4 agonist, or sirtuin-activating compound normally administered in a single dosage (monotherapy) to produce an anti-inflammatory effect. Preferably, the amount of PPAR agonist (e.g., PPARα, PPAR β/δ, or PPARγ agonist), LXR agonist (e.g., LXRα or LXRβ agonist), RXR agonist (e.g., RXRα, RXRβ, or RXRγ agonist), HNF-4 agonist, or sirtuin-activating compound is less than 90%, more preferably less than 80%, and most preferably, less than 70% of the recommended monotherapy dosage amount. The amount of compound of formula A, compound of any one of formulae 1-49 or I-III, lipoxin compounds, oxylipin compound, or combination of aspiring and an omega-3 fatty acid in the combination composition of this invention is less than 5%, less than 10%, less than 15%, less than 20%, less than 25%, less than 30%, less than 40%, less than 50%, less than 60%, less than 70%, less than 80%, less than 90%, or less than 100% of the dose of compound of formula A, compound of any one of formulae 1-49 or I-III, lipoxin compound, oxylipin compounds, or combination of aspirin and an omega-3 fatty acid administered in a single dosage or produce an anti-inflammatory effect. Preferably, the amount of compound of formula A, compound of any one off formulae 1-49 or I-III, lipoxin compound, oxylipin compound, or combination of aspirin and an omega-3 fatty acid is less than 100%, preferably less than 90%, more preferably less than 80% and most preferably, less than 7% of the dose of compound of formula A, compound of any one of formulae 1-49 or I-III, lipoxin compound, oxylipin compound, or combination of aspirin and an omega-3 fatty acid administered in a single dosage (i.e., without a PPAR, LXR, RXR, or HNF-4 agonist, or a sirtuin-activating compound) to produce an anti-inflammatory effect.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of formula A, compound of any one of formulae 1 -49 or I-III, lipoxin compound, oxylipin compound, or aspirin and/or an omega-3 fatty acid and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery systems, e.g., a skin patch.

A pharmaceutically acceptable carrier can contain physiologically acceptable agents that act, for example, to stabilize or to increase the absorption of a compound such as a compound of formula A, compound of any one of formulae 1-49 or I-III, lipoxin compound, oxylipin compound, or aspirin and/or an omega-3 fatty acid. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated there, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulation may conventiently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of formula A, compound of any one of formulae 1-49 or I-III, lipoxin compound, oxylipin compound, or aspirin and/or an omega-3 fatty acid, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulation of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures hereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluents, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agent, emulsifying and suspending agents, sweetening, flavoring, coloring, perfuming and preservative agents.

Suspension in addition to the active compounds, may contain suspending agents, for example, ethoxylated cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with on or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administrations also include pessaries, tampons, creams, gels, pastes, foams or spray formulations containing such carriers as are known in the art to the appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contains, in addition to an active compound, excipients such as lactose, talc, silicic acid, aluminum hydroxide, calcium silicates and polyamide powder, or mixtures of these substances. Sprays can additionally contain customary propellants, such as chlorfluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administrations other than enteral and topical administration, usually by injection, and includes, without limitation, intravenous, intramuscular, intraarterial, intrathecal, intracapsular, intraobital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples or suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic the patient.

The selected dosage level will depend upon a variety of factors including the activity of the ;particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physical or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such ah effective dose will generally depend upon the doctors described above.

It desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including, primates, In particular humans, and other mammals such as equines, cattle, swine and sheep; and poultry and pets in general.

In certain embodiments, the method of treating inflammatory disease comprises conjointly administering: a) a compound of formula A, compound of any one of formulae 1-49 or I-III, lipoxin compound, oxylipin compound, or combination of aspirin and an omega-3 fatty acid; with b) a PPAR agonist (e.g., a PPARα, PPAR β/δ, or a PPARγ agonist), LXR agonist (e.g., LXRα or LXRβ agonist), an RXR agonist (e.g., an RXRα, RXRβ, or an RXRγ agonist), an HNF-4 agonist, or a sirtuin-activating compound; and optionally conjointly with c) another therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, ether concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In one embodiment, the method of treating inflammatory disease according to this invention may comprise the additional step of conjointly administering to the patient another anti-inflammatory agent including, for example, a non-steroidal anti-inflammatory drug (NSAID), a mast cell stabilizer, or a leukotriene modifier.

In certain embodiments, the methods of treating a complex disorder having an inflammatory component, such as type 2 diabetes, or of treating type 1 diabetes according to this invention may comprise the additional step of conjointly administering to the patient another treatment for diabetes including, but not limited to, sulfonylureas (e.g., chlorpropamide, tolbutamide, glyburide, glipizide, acetohexamide, tolazamide, gliclazide, gliquidone, or glimepiride), medications that decrease the amount of glucose produced by the liver (e.g., metformin), meglitinides, (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, or combinations of the above.

In certain embodiments, the methods of treating a complex disorder having an inflammatory component, such as obesity, according to this invention may comprise the additional step of conjointly administering to the patient another treatment for obesity including, but not limited to, orlistat, sibutramine, phendiametrazine, rimonabant, cetilistat, G 389-255, APD356, pramlintide/AC137, PYY3-36, AC 162352/PYY3-36, oxyntomodulin, TM 30338, AOD 9604, oleoyl-estrone, bromocriptine, ephedrine, leptin, pseudoephedrine, or pharmaceutically acceptable salts thereof.

In certain embodiments, the use of a composition comprising both a compound of formula A, compound of any one of formulae 1-49 or I-III, lipoxin compound, oxylipin compound, or a combination of aspirin and an omega-3 fatty acid and a PPAR agonist (e.g., a PPARα, PPARβ/δ, or a PPARγ agonist), an LXR agonist (e.g., LXRα or LXRβ agonist), an RXR agonist (e.g., an RXRα, RXRβ, or an RXRγ agonist), an HNF-4 agonist, or a sirtuin-activating compound according to this invention in the treatment of inflammatory disease, does not preclude the separate but conjoint administration of another PPAR agonist (e.g., a PPARα, PPARβ/δ, or PPARγ agonist), LXR agonist (e.g., LXRα or LXRβ agonist), RXR agonist (e.g., an RXRα, RXRβ, or an RXRγ agonist), HNF-4 agonist, or a sirtuin-activating compound.

In certain embodiments, different compounds of formulae A, compounds of any one of formulae 1-49 or I-III, lipoxin compounds, or oxylipin compounds may be conjointly administered with one another while conjointly administering a PPAR agonist, (e.g., a PPARα, PPARβ/δ, or a PPARγ agonist), LXR agonist (e.g., an LXRα or LXRβ agonist), an RXR agonist (e.g., an RXRα, RXRβ, or an RXRγ agonist), an HNF-4 agonist, or a sirtuin-activating compound. Moreover, such combinations may be conjointly administered with other therapeutic agents, such as other anti-inflammatory agents. In certain embodiments, different compounds of formulae A, compounds of any of formulae 1-49 or I-III, lipoxin compounds, or oxylipin compounds may be conjointly administered a PPAR agonist (e.g., a PPARα, PPARβ/δ, or a PPARγ agonist), LXR agonist (e.g., an LXRα or LXRβ agonist), an RXR agonist (e.g., an RXRα, RXRβ, or an RXRγ agonist), an HNF-4 agonist, or a sirtuin-activating compound. Such combinations may further be conjointly administered with other therapeutic agents, such as other anti-inflammatory agents.

In embodiments where a combination of aspirin and an omega-3 fatty acid are administered the aspirin and omega-3 fatty acid can be administered simultaneously, e.g., as a single formulation comprising both components or in separate formulations, or can be administered at separate times, provided that, at least at certain times during the therapeutic regiment, both the aspiring and omega-3 fatty acid are present simultaneously in the patient at levels that allow the omega-3 fatty acid to be metabolized as described in Serhan, et. al., 2002, J. Exp. Med., 196: 1025-1037. In certain such embodiments, the omega-3 fatty acid is provided in the form of a partially purified natural extract, such as fish oil, while in other embodiments, the omega-3 fatty acid may be provided as a substantially pure preparation of one or more omega-3 fatty acids, such as a C18:3, C20:5, or C22:6 fatty acid, particularly eicosapentaenoic acid or docosahexaenoic acid. A substantially pure preparation of one or more omega-3 fatty acids refers to a composition wherein the fatty acid component is at least 90%, at least 95%, or even at least 98% of ne or more omega-3 fatty acids, such as one or more specified omega-3fatty acids. Non-fatty acid components, such as excipients or other materials added during formulation, are not considered for the purpose of determining whether the fatty acid component meets the desired level of purity.

In certain embodiments, COX-2 inhibitor other than aspirin, such as celecoxib, rofecxib, valdecoxib, lumiracoxib, etoricoxib, NF-398, or parecoxib, may be used in combination with an omega-3 fatty acid for the treatment of inflammatory disease in any of the various embodiments discussed herein. In certain embodiments, a non-selective NSAID other than aspirin, such as diclofenac, diflunisal, etodolac, fenoprofen, ibuprofen, indomethacin, ketoprofen, ketorolac, mefenamic acid, meloxicam, nabumetone, naproxen, oxaprozin, piroxicam, salsalate, sulindae or tolmetin, may be used in combination with an omega-3 fatty acid for the treatment of inflammatory disease in any of the various embodiments discussed herein. The combination of different COX-2 inhibitors or non-selective NSAIDs with an omega-3 fatty acid may result in the production of different subsets or proportions of active omega-3 metabolites.

This invention includes the use of pharmaceutically acceptable salts of compounds of formula A, compounds of any one of formulae 1-49 or I-III, lipoxin, compounds, or oxylipin compounds and/or PPAR agonists (e.g., a PPARα, PPARβ/δ, or a PPARγ agonists), LXR agonists (e.g., LXRα or LXRβ agonists), RXR agonists (e.g., an RXRα, RXRβ, or an RXRγ agonists), HNF-4 agonists, or a sirtuin-activating compounds in the compositions and methods of the presented invention. In certain embodiments, contemplated salts of the invention include alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium hydroxide, L-lysine, magnesium hydroxide, 4-(2-hydroxyethyl)morpholine, piperazine, potassium hydroxide, 1-(2-hydroxyethyl)pyrrolidine, sodium hydroxide, triethanolamine, tromethamine, and zinc hydroxide salts. In certain embodiments, contemplated salts of the invention include Na, Ca, K, Mg, Zn or other metal salts.

The pharmaceutically acceptable acid addition salts can also exist as various solvates, such as with water, methanol, ethanol, dimethylformamide, and the like. mixtures of such solvates can also be prepared. The source of such solvate can be from the solvent of crystallization, inherent in the solvent of preparation or cystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

### EXAMPLES

Unless otherwise noted, reagents and solvents were used as received from commercial suppliers. Proton nuclear magnetic resonance spectra were obtained on a Bruker AVANCE 300 spectrometer at 300 MHz or on a Bruker ADVANCE 500 spectrometer at 500 MHz. Spectra are given in ppm (δ) and coupling constants/values, are reported in Hertz. Tetramethylsilane was used as an internal standard. Mass spectra were obtained on a Perkin Elmer Sciex 100 atmospheric pressure ionization (APCI) mass spectrometer, or a Finnigan LCQ Duo LC-MS ion trap electrospray ionization (ESI) mass spectrometer. Thin-layer chromatography (TLC) was performed using Analtech silica gel plates, EMD silica gel 60 F₂₅₄ or SAI plastic backed silica gel plates and visualized by ultraviolet (UV) light, iodine, ceric ammonium molybdate or potassium permanganate solution. HPLC analyses were obtained using a BDS C18 column (4.6×250 mm) with UV detection at 254 nm using standard solvent gradient programs (Method 1 and Method 2). Preparative HPLC purifications were performed using a Luna C18 column (21.2×150 mm) with UV detection at 254 nm using various solvent gradient programs and isocratic elutions as described.

### Method 1:

| Time (min) | Flow (mL/min) | % A | % B |
|---|---|---|---|
| 0.0 | 1.0 | 90.0 | 10.0 |
| 20.0 | 1.0 | 0 | 100.0 |
| 35.0 | 1.0 | 0 | 100.0 |

A = Water with 0.05% v/v Trifluoroacetic Acid
B = Acetonitrile with 0.05% v/v Trifluoroacetic Acid.

Additional details on the synthesis of compounds of formulae I-III can be Founds in U.S. Provisional Patent Application No. 61/194,093 filed on Sep. 23, 2008, entitled “Therapeutic Compounds,” to Schwartz.

### Example 1

### Synthesis of Bromoallylic Alcohol Reagent 403

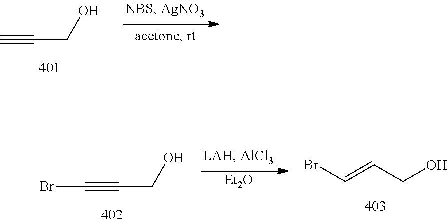

A mixture of propargyl alcohol (401: 3.26 g, 58.3 mmol), N-bromosuccinimide (11.2 g, 62.9 mmol) and silver(I) nitrate (1.00 g, 5.88 mmol) in acetone (100 mL) was stirred at room temperature for 2 h. After this time, the reaction mixture was concentrated and the residue redissolved in iced water (150 mL) and diethyl ether (200 mL), the aqueous layer was removed and extracted with diethyl ether (100 mL). The combined organic layers were washed with brine (100 mL) and dried over sodium sulfate, filtered and concentrated, to given 7.83 g of bromoproargylic alcohol 402 as an orange/yellow oil which was used crude in the next step.

A solution of aluminum trichloride (7.70 g, 57.7 mmol) in diethyl ether (40 mL) was added dropwise to a stirred suspension of lithium aluminum hydride (4.38 g, 115 mmol) in diethyl ether (40 mL) at −5° C. followed by the careful addition of bromoprogargylic alcohol (402; 7.38 g, from step 1). The mixture was warmed to room temperature and heated at reflux for 3 h. After this time, the reaction was cooled to room temperature and then to −5° C. Water (4.4 mL) was added carefully and then the reaction mixture was diluted with diethyl ether (100 mL). Sodium hydroxide (15% aqueous, 4.4 mL) was added carefully, followed by water (13 mL). Diethyl ether (100 mL) and magnesium sulfate (10 g) were added, the mixture was stirred for 5 min and then filtered through diatomaceous earth and the filter cake then rinsed with diethyl ether (3×100 mL) and the combined filtrates concentrated. Purification by vacuum distillation (85-100° C. 150 mmHg) afforded the desired bromoallylic alcohol product 403 (3.90 g, 50%) as a colorless oil

### Example 2

### Synthesis of Phosphonate Building Blocks 411 and 411a

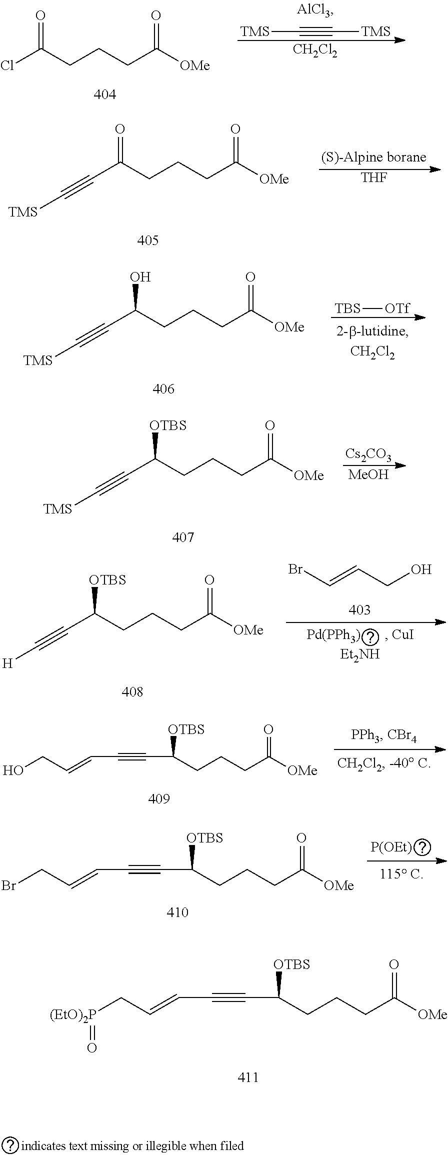

Synthesis of Compound 405. A solution of methyl 4-(chlorocarbonyl)butanoate (404, 23.0 g, 139 mmol) in methylene chloride (40 mL) was added dropwise over 10 min to a suspension of aluminum chloride (22.3 g, 167 mmol) in methylene chloride (130 mL) at 0° C. The mixture was stirred at 0° C. for 3 h. then poured into a mixture of ice (150 mL) and 0.1 N HCl (150 mL), stirred for 5 min and then diluted with diethyl ether (450 mL) and water (100 mL). The aqueous layer was separated and extracted with diethyl ether (2×150 mL). The combined organic layers were washed with water (300 mL), saturated aqueous sodium bicarbonate (300 mL) and brine (300 mL), dried over sodium sulfate, filtered and concentrated. Purification by flash chromatography (silica, 90:10 hexanes/ethyl acetate) afforded ketone 405 (16.1 g, 51%) as an orange oil.

Synthesis of Compound 406. A mixture of 9-borabicyclo[3.3.1]nonane (26.9 g, 100 mmol) and (1S)-(−)-α-pinene (S-alpine borane; 33.0 g, 242 mmol) was stirred at 65° C. for 3.5 h. The solution was cooled to 0° C. and then a solution of 405 (15.0 g, 66.3 mmol) in tetrahydrofuran was added over 5 min. The reaction was stirred at 0° C. for 20 min and then allowed to warm to room temperature and stirred overnight. The solution was then cooled to 0° C. and acetaldehyde (10.0 mL, 178 mmol) was added and the mixture heated under vacuum at 65° C. for 1 h. The resulting residue was diluted with diethyl ether (120 mL), cooled to 0° C. and stirred with ethanolamine (6.07 g, 99.4 mmol) for 5 min. The cooling bath was removed and the mixture was stirred for an additional 30 min. The resulting precipitate was removed by filtration and the filtrate concentrated to afford a deep yellow oil. Purification by flash chromatography (silica, hexanes to 85:15 hexanes/ethyl acetate) afforded compound 406 (11.9 g, 78%) as a light yellow oil.

Synthesis of Compound 407. To a stirred solution of 406 (12.1 g, 52.9 mmol) in dry methylene chloride (250 mL) at 0° C. under nitrogen was added 2,6-latidine (12.5 g, 116 mmol). The mixture was stirred for 5 min and then tert-butyldimethylsilyl trifluoromethanesulfonate (20.9 g, 79.5 mmol) was added over 5 min. The reaction was then warmed to room temperature and stirred overnight. The reaction was quenched by adding a saturated aqueous ammonium chloride solution (130 mL), the aqueous layer was separated and then extracted with diethyl ether (2×200 mL). The combined organic layers were dried over magnesium sulfate, filtered and concentrated. Purification by flash chromatography (silica, hexanes to 9:1 hexanes/ethyl acetate) afforded 407 (17.0 g, 93%) as a yellow oil.

Synthesis of Compound 408. To a stirred solution of 407 (14.9 g, 43.5 mmol) in dry methanol (225 mL) under nitrogen was added cesium carbonate (28.3 g, 86.9 mmol) and the mixture was stirred for 25 min. After this time, the reaction was diluted with water (200 mL) and extracted with diethyl ether (3×300 mL). The combined organic layers were dried over magnesium sulfate, filtered and concentrated. Purification by flash chromatography (silica, hexanes to 3:1 hexanes/ethyl acetate) afforded 408 (11.1 g, 94%) as a yellow oil.

Synthesis of Compound 409. To a stirred solution of bromoallylic alcohol (403: 3.44 g, 25.1 mmol) in degassed diethylamine (13 mL) under argon, was added terakis(triphenylphosphino) palladium(0) (0.29 g, 0.25 mmol), followed by a solution of 408 (6.78 g, 25.1 mmol) in degassed diethylamine (25 mL). Copper(I) iodide (0.24 g, 1.25 mmol) was added and the reaction mixture stirred for 16 h at room temperature. The reaction mixture was then diluted with diethyl ether (350 mL) and washed with water (4×125 mL) and brine (2×100 mL), dried over sodium sulfate filtered and concentrated. Purification by flash chromatography (silica, hexanes to 3:1 hexanes/ethyl acetate) afforded 409 (7.33 g, 90%) as an orange oil.

Synthesis of Compound 410. Triphenylphosphine (7.65 g, 29.2 mmol) was added to a stirred solution of 409 (7.33 g, 22.4 mmol) in methylene chloride (250 mL) at −40° C. Carbon tetrabromide (8.92 g, 26.9 mmol) was then added and the mixture maintained between −35 to −45° C. for 1 h. After this time, the reaction mixture was diluted with diethyl ether (500 mL) and saturated aqueous sodium bicarbonate (250 mL). The organic layer was removed and washed with water (200 mL) and brine (200 mL), dried over sodium sulfate, filtered and concentrated. Purification by flash chromatography (silica, hexanes to 5:1 hexanes/ethyl acetate) afforded 410 (7.89 g, 90%) as a pale yellow oil.

Synthesis of Compound 411. A mixture of 410 (7.89 g, 20.2 mmol) and triethylphosphite (30 mL) was heated at 115° C. for 2 h. The reaction was then cooled to room temperature and concentrated in vacuo. Purification by flash chromatography (silica, 5:1 to 1:4 hexanes/ethyl acetate) afforded 411 (8.33 g, 92%) as a pale yellow oil.

Synthesis of Compound 411a. The ethyl ester equivalent of the phosphonate building block 411a was similarly prepared substituting ethyl 4-(chlorocarbonyl)butanoate, for methyl 4-(chlorocarbonyl)butanoate as reagent 404.

### Example 3

### Synthesis of (S)-ethyl 5-hydroxyhept-6-ynoate 412

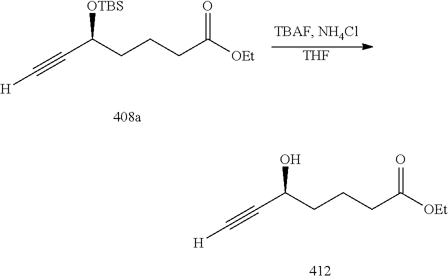 [SMILES: C#C[C@H](CCCC(=O)OCC)O[Si](C)(C)C(C)(C)C ; C#C[C@@H](O)CCCC(=O)OCC ; "THF"]

Compound 408a (26.7 g, 104 mmol) and ammonium chloride (5× molar excess) were dissolved in terahydrofuran (15 mL) at 0° C. and the tetrabutylammonium floride (5× molar excess of a 1.0 M solution in tetrahydrofuran) was added. The reaction mixture was stirred for 2 h at room temperature. After this time, the reaction was diluted with water (20 mL) and extracted with diethyl ether (2×45 mL). The combined organic layers were washed with brine, dried over sodium sulfate, filtered and concentrated. Purification by silica plug filtration (silica, 95:5 to 80:20 hexanes/ethyl acetate) afforded 412 (15.9 g, 83%) as a yellow oil.

### Example 4

### Synthesis of Aldehyde Building Block 418

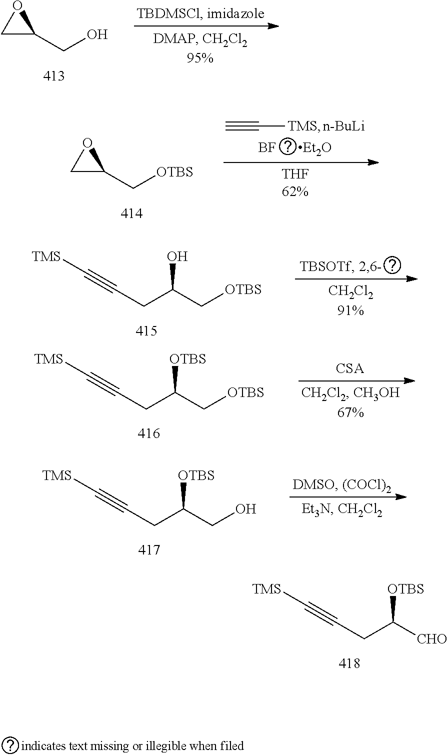

Synthesis of Compound 414. To a solution of commercially available (S)-glycidol (413, 5.10 g, 68.8 mmol) in methylene chloride (40 mL) at 0° C. was added imidazole (6.10 g, 89.5 mmol), followed by 4-dimethylaminopyridine (0.420 g, 3.40 mmol), and then stirred at 0° C. for 15 min. A solution of tert-butylchlorodimethylsilane (10.4 g, 68.8 mmol) in dry methylene chloride (20 mL) was then added dropwise over 5 min. The reaction was stirred at 0° C. for 20 min and then at room temperature for 1 h. After this time, the mixture was quenched with water (100 mL), diluted with diethyl ether (300 mL) and the layers were separated. The aqueous layer was extracted with diethyl ether (2×200 mL) and the combined organic layers were dried over magnesium sulfate, filtered and concentrated. Purification by flash chromatography (silica plug, hexanes to 95.5 hexanes/ethyl acetate) afforded 414 (11.8 g, 92%) as a light yellow oil.

Synthesis of Compound 415. To a stirred solution of trimethysilylacetylene (4.17 g, 42.5 mmol) in tetrahydrofuran (76 mL) at −78° C. under nitrogen was added a solution of n-butyl lithium in tetrahydrofuran (1.41 M, 15.0 mL, 21.2 mmol) over 10 min. The reaction was stirred at −78° C. for 30 min then a solution of 414 (4.00 g, 21.2 mmol) in tetrahydrofuran (15 mL) was then added, followed by boron trifluoride diethyl etherate (3.10 g, 21.2 mmol). The mixture was then stirred at −78° C. for 30 min and then at room temperature for 1 h. After this time the reaction was quenched by adding a saturated aqueous ammonium chloride solution (40 mL) then diluted with diethyl ether (400 mL). The organic layer was separated, washed with brine (250 mL) and concentrated. Purification by flash chromatography (silica, hexanes to 22.3 hexanes/ethyl acetate) afforded 415 (5.13 g, 84%) as a colorless oil.

Synthesis of Compound 416. To a stirred solution of 415 (6.44 g, 22.4 mmol) in methylene chloride (65 mL) at 0° C. under nitrogen was added 2,6-lutidine (5.29 g, 49.4 mmol) and them mixture stirred for 10 min. Tert-butyldimethylsilyl trifluoromethanesulfonate (8.92 g, 33.7 mmol) was then added slowly over 10 min and the solution was allowed to warm to room temperature overnight. Saturated aqueous ammonium chloride solution (40 mL) was added, then the aqueous layer was separated and then extracted with diethyl ether (200 mL). The combined organic layers were dried over magnesium sulfate, filtered and concentrated. Purification by flash chromatography (silica, hexanes to 9:1 hexanes/ethyl acetate) afforded 416 (8.71 g, 96%) as a light yellow oil.

Synthesis of Compound 417. To a stirred solution of 416 (9.20 g, 22.9 mmol) in methylene chloride (110 mL) and methanol (110 mL) at −5° C. under nitrogen was added (±)-camphor-10-sulfonic acid (5.33 g, 22.9 mmol) and the mixture stirred for 20 min. After this time, the reactions was quenched by adding triethylamine (15 mL) and then concentrated. Purification by flash chromatography (silica, hexanes to 19:1 hexanes/ethyl acetate) afforded 417 (4.41 g, 67%) as a light yellow oil.

Synthesis of Compound 418. To a stirred solution of oxalyl chloride (3.00 g, 23.6 mmol) in methylene chloride (25 mL) at −78° C. under nitrogen was added dropwise a solution of dimethyl sulfoxide (2.20 mL, 30.7 mmol) in methylene chloride (35 mL) followed by stirring at −78° C. for 10 min. A solution of 417 (4.40 g, 15.3 mmol) in methylene chloride (45 mL) was then added and the mixture at −78° C. for 1 h, followed by the addition of triethylamine (7.76 g, 76.7 mmol). The dry ice bath was removed and the reaction was stirred for 45 min. After this time, the reaction was diluted with water (30 mL), the aqueous layer separated and then extracted with diethyl ether (200 mL). The combined organic layers were dried over magnesium sulfate, filtered and concentrated. Purification by flash chromatography (silica, hexanes to 19:1 hexanes/ethyl acetate) afforded 418 (3.91 g, 89%) as a light yellow oil.

### Example 5

### Synthesis of Aldehyde Intermediate 432

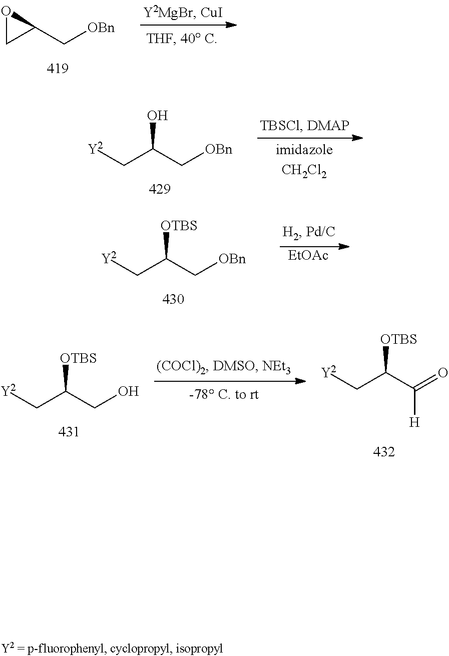

Synthesis of Compound 429. A mixture of finely crushed copper(I) iodide (10%) molar amount) and tetrahydrofuran (500 mL) was cooled to −78° C. and a solution of the appropriate magnesium bromide (5× molar excess) was added dropwise over a period 30 min. Compound 419 in tetrahydrofuran (60 mL) was then added dropwise over a period of 20 min and the reaction mixture was stirred −78° C. for 45 min. The reaction was cautiously quenched with saturated aqueous ammonium chloride (300 mL) and then allowed to warm to room temperature. The aqueous layer was separated and extracted with diethyl ether (2×200 mL). The combined organic layers were washed with brine (200 mL), dried over sodium sulfate filtered and concentrated. Purification by flash chromatography (silica, hexanes to 80:15 hexanes/ethyl acetate) afforded 429.

Synthesis of Compound 430. To a stirred solution of compound 429 in methylene chloride (60 mL) at 0° C. was added a 5% molar amount of 4-dimethylaminopyridine, a 1.25× molar excess of imidazole and a molar a equivalent of tert-butyldimethylsilyl chloride. The cooling bath was removed and the reaction stirred at room temperature for 3 h. After this time, the mixture was quenched with water (75 mL) and extracted with diethyl ether (2×100 mL). The combined organic layers were washed with brine (100 mL), dried over sodium sulfate, filtered and concentrated. Purification by flash chromatography (silica, hexanes to 96:4 hexanes/ethyl acetate) afforded 430.

Synthesis of Compound 431. Palladium on carbon (10 wt % (dry bases), 50% water) was added to compound 430 in ethyl acetate and shaken under an atmosphere of hydrogen (50 psi) at room temperature until hydrogen uptake had ceased. The reaction mixture was filtered through diatomaceous earth, and the filter cake was washed with ethyl acetate (800 mL). Purification by flash chromatography (silica, hexanes to 80:30 hexanes/ethyl acetate) afforded 431.

Synthesis of Compound 432. Oxalyl chloride (1.5% molar excess) was added dropwise to a stirred solution of dimethyl sulfoxide (2× molar excess) in methylene chloride (70 mL) under nitrogen at −78° C. The reaction mixture was stirred at −78° C. for 5 min before a solution of compound 431 (5.00 g, 24.5 mmol) in methylene chloride (30 mL) was added over a period of 10 min. The mixture was stirred at −78° C. for 115 min and then triethylamine (4.75× molar excess) was slowly added. The dry ice bath was removed and the reaction was stirred for 90 min. After this time water (220 mL) was added, the aqueous layer was separated and then extracted with diethyl ether (2×300 mL). The combined organic layers were dried over sodium sulfate, filtered and concentrated. Purification by flash chromatography afforded 432.

### Example 6

### Synthesis of Compounds 303, 304, 205, 326, 327 and 328

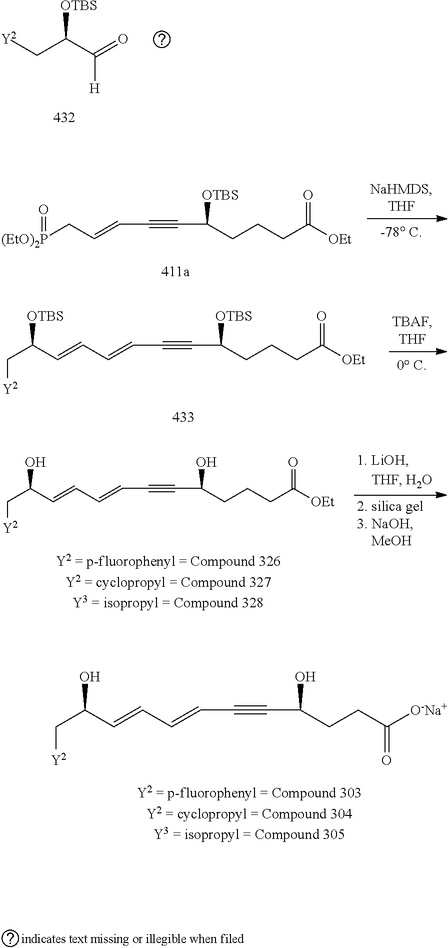

Synthesis of Compound 433. To a stirred solution of phophonate 411a in tetrahydrofuran (80 mL) at −78° C. was added sodium (bistrimethysilyl)lamide (7.6 mL of a 1.0 M solution in tetrahydrofuran) over 15 min. A solution of the appropriate aldehyde 432 (1.7× molar excess) in terahydrofuran (20 mL) was added immediately. The resulting solution was stirred at −78° C. for 2 h and then allowed to warm slowly to 0° C. over 14 h. The reaction was then diluted with diethyl ether (300 mL) and 10% aqueous ammonium chloride solution (100 mL) .The aqueous layer was separated and extracted with diethyl ether (2×50 mL). the combined organic layers were washed with brine (100 mL), dried over sodium sulfate, filtered, and concentrated. Purification by flash chromatography (silica, hexanes to 96:4 hexanes/ethyl acetate) afforded 433.

Syntheses of Compounds 326, 327 and 328. Compound 433 was deprotected as described in Example 3 for the synthesis of Compound 412.

Compound 326 was purified by flash chromatography (silica, methylene chloride to 96:4 methylene chloride/methanol) resulting in 78% yield; ¹H NMR (500 MHz, CD₃OD) δ 7.21 (dd, J=8.5, 5.5 Hz, 2H), 7.00-6.93 (m, 2H), 6.53 (dd, J=15.5, 10.8 Hz, 1H) 6.20 (dd, J=15.2, 10.8 Hz, 1H), 5.82 (dd, J=15.2, 6.5 Hz, 1H), 5.62 (d, J=15.5 Hz, 1H), 4.43 (t, J=6.5 Hz, 1H) 4.30 (q, J=6.5 Hz, 1H) 4.12 (q, J=7.1 Hz, 2H), 2.90-2.70 (m, 2H), 2.36 (t, J=7.0 Hz, 2H), 1.80-1.62 (m, 4H), 1.23 (t, J=7.1 Hz, 3H).

Compound 327 was purified by flash chromatography (silica, methylene chloride to 96:4 methylene chloride/methanol) resulting in 59% yield: ¹H NMR (500 MHz, CDCl₃) δ 6.58 (dd, J=15.5, 10.8 Hz, 1H), 6.29 (dd, J=15.2, 10.8 Hz, 1H), 5.86 (dd, J=15.2, 6.5 Hz, 1H), 5.66 (d, J=15.6 Hz, 1H), 4.44 (td, J=6.5, 1.6 Hz, 1H), 4.18 (q, j=6.5 Hz, 1H), 4.12 (q, J=7.1 Hz, 2H), 2.36 (t, J=7.0 Hz, 2H), 1.80-1.62 (m, 4H), 1.53-1.45 (m, 1H), 1.35-1.28 (m, 1H), 1.24 (t, J=7.1 Hz, 3H), 0.80-0.70 (m, 1H), 0.50-0.40 (m, 2H), 0.13-0.00 (m, 2H), HPLC (Method 1) t_{R}=14.8 min., 86.4% (AUC).

Compound 328 was purified by RP preparative chromatography (57:43 to 60:40 methanol/water) resulting in 30% yield: ¹H NMR (500 MHz, CDCl₃) δ 6.56 (dd, J=15.5, 10.8 Hz, 1H), 6.28 (dd, J=15.2, 10.8 Hz, 1H), 5.78 (dd, J=15.2, 6.5 Hz, 1H), 5.66 (d, J=15.6 Hz, 1H), 4.44 (td, J=6.5, 1.6 Hz, 1H), 4.16 (q, J=6.5 Hz, 1H), 4.12 (q, J=7.2 Hz, 2H), 2.36 (t, J=7.0 Hz, 2H), 1.80-1.62 (m, 5H), 1.47-1.41 (m, 1H), 1.32-1.21 (m, 4H), 0.92 (dd, J=6.6, 1.8 Hz, 6H).

Syntheses of Compounds 303, 304 and 305. Lithium hydroxide (2× molar excess) was added to a solution of the appropriate methyl ester in tetrahydrofuran (1.6 mL) and water (0.4 mL). After stirring at room temperature for 15 h, the reaction mixture was concentrated and run through a small silica plug (silica, dichloromethane To 85:15 dichloromethane/methanol). The resulting free acid was dissolved in Methanol (2 mL) and sodium hydroxide (0.1 M in methanol, 1.35 mL) was added. The solution was concentrated to provide compound the desired sodium salt.

Compound 303 was produced in 95% yield: ¹H NMR (500 MHz, CD₃OD) δ 7.20 (dd, J=8.5, 5.5 Hz, 2H), 7.00-6.93 (m, 2H), 6.52 (dd, J=15.5, 10.8 Hz, 1H), 6.19 (dd, J=15.3, 10.8 Hz, 1H), 5.80 (dd, J=15.2, 6.5 Hz, 1H), 5.60 (dd, J=15.5 Hz, 1H), 4.45-4.41 (m, 1H) 4.29 (q, J=6.0 Hz, 1H), 2.85-2.70 (m, 2H) 2.19 (t, J=7.5 Hz, 2H), 1.78-1.60 (m 4H); ESI MS m/z 335 [M+Na]⁺; HPLC (Method 1) l_{R}=13.3 min., 97.9% (AUC).

Compound 304 was produced in 90% yield: ¹H NMR (500 MHz, CD₃OD) δ 6.56 (dd, J=15.5, 10.8 Hz, 1H), 6.28 (dd, J=15.3, 10.8 Hz, 1H), 5.85 (dd, J=15.2, 6.5 Hz, 1H), 5.64 (d, J=15.5 Hz, 1H), 4.46-4.39 (m, 1H) 4.20-4.14 (m, 1H), 2.19 (t, J=7.1 Hz, 2H), 1.80-1.63 (m, 4H) 1.53-1.45 (m, 1H), 1.35-1.28 (m, 1H), 0.80-0.70 (m, 1H) 0.49-0.38 (m, 2H), 0.11-0.00 (m, 2H); ESI MS m/z 279 [M+Na]⁺; HPLC (Method 1) l_{R}=12.1 min., 98.7% (AUC).

Compound 305 was produced in 95% yield: ¹H NMR (500 MHz, CD₃OD) δ 6.55 (dd, J=15.5, 10.8 Hz, 1H), 6.26 (dd, J=15.2, 10.8 Hz, 1H), 5.78 (dd, J=15.2, 6.6 Hz, 1H), 5.64 (d, J=15.6 Hz, 1H), 4.46-4.41 (m, 1H), 4.15 (q, J=6.5 Hz, 1H), 2.19 (t, J=7.1 Hz, 2H), 1.80-1.64 (m, 5H), 1.48-1.40 (m, 1H), 1.32-1.24 m, 1H), 0.92 (dd, J=6.6, 2.0 Hz, 6H); ESI MS m/z 279 [M−H]:HPLC (Method 1) t_{R}=13.0 min., 98.9% (AUC).

### Example 7

### Synthesis of Aldehyde Intermediate 437

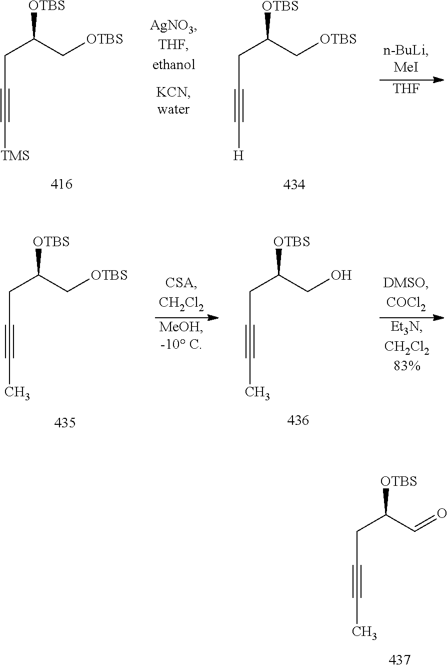

Synthesis of Compound 434. To a stirred solution of compound 416 (1.03 g, 2.56 mmol) in tetrahydrofuran (12.9 mL) and absolute ethanol (6.5 mL) at 0° C. under nitrogen was added dropwise a solution of silver(1) nitrate (0.689 g, 4.06 mmol) in tetrahydrofuran (6.5 mL) and water (6.5 mL) and a yellow precipitate was formed. The ice-bath was replaced with a room temperature water bath and the reaction mixture was stirred for 1.5 h. The reaction mixture was then cooled to 0° C. and a solution of potassium cyanide (0.451 g, 6.92 mmol) in water (6.5 mL) was added dropwise. The ice-bath was removed and the reaction was stirred for 15 min and then filtered through diatomaceous earth. The filter cake was washed with diethyl ether (50 mL), water (50 mL) then with ethyl acetate (50 mL) and finally with water (50 mL). The aqueous layer of the filtrate was separated and extracted with ethyl acetate (50 mL). The combined organic layers were washed with brine (50 mL), dried over magnesium sulfate, filtered and concentrated. Purification by flash chromatography (silica, 95.5 hexanes/ethyl acetate) afforded 434 (0.64 g, 76%) as colorless oil.

Synthesis of Compound 435. To a solution of 434 (0.504 g, 1.53 mmol) in anhydrous tetrahydrofuran (15 mL) at −78° C. was added dropwise n-butyl lithium (1.04 mL, 1.77 M in hexanes). After stirring for 25 min, iodomethane (0.19 mL, 3.06 mmol) was added and then the reaction mixture was allowed to warm slowly to room temperature. After stirring for a further 6 h, the reaction was quenched by the addition of aqueous ammonium chloride. The mixture was extracted with ether (2×50 mL), and the organic layers were combined, dried over sodium sulfate, filtered, and concentrated. Purification by flash chromatography (silica, 95:5 hexanes/ethyl acetate) afforded 435 (0.449 g, 85%) as a light yellow oil.

Synthesis of Compound 436. Compound 435 was deprotected to form compound 436 as described in Example 4 for the production of Compound 417. purification by flash chromatography (silica, hexanes to 95.5 hexanes/ethyl acetate) afforded 436 in 55% yield.

Synthesis of Compound 437. Compound 436 was oxidized to Compound 437 as described in Example 4 for the production of Compound 418. Purification by flash chromatography (silica, 94:6 hexanes/ethyl acetate) afforded 437 in 83% yield.

### Example 8

### Synthesis of 1,2-dichloro-4-ethynylbenzene 400

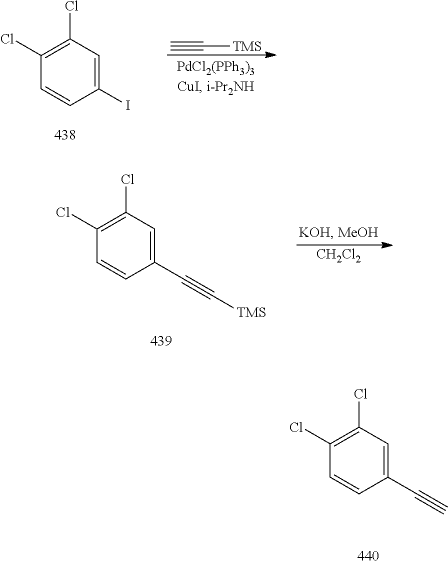

Synthesis of Compound 439. A mixture of 438 (5.03 g, 18.4 mmol), (bistriphenylphosiphino)palladium(II) chloride (0.323 g, 0.461 mmol) and copper(I) iodide (0.088 g, 0.461 mmol) in diisopropylamine (40 mL) was heated to 40° C. and trimethylsilyl acetylene (1.99 g, 20.2 mmol) was added. After stirring at 40° C. for 18 h, the reaction mixture was cooled to room temperature, poured into water (120 mL) and then extracted with methylene chloride (3×40 mL). The combined organic extracts were dried over magnesium sulfate, filtered and concentrated. Purification by flash chromatography (silica, hexanes) afforded 439.

Synthesis of Compound 440. A mixture of 439 (2.54 g, 10.4 mmol) and potassium hydroxide (1.17 g, 20.9 mmol) in methanol (20 mL) and methylene chloride (10 mL) was stirred at room temperature for 1 h. After this time, the reaction mixture was poured into water (30 mL) and extracted with methylene chloride (3×30 mL). The combined organic extracts were dried over magnesium sulfate, filtered and concentrated to afford 440 (1.68 g, 94%) which was used as an appropriate alkyne if Example 9 without further purification.

### Example 9

### Synthesis of Aldehyde Intermediate 444

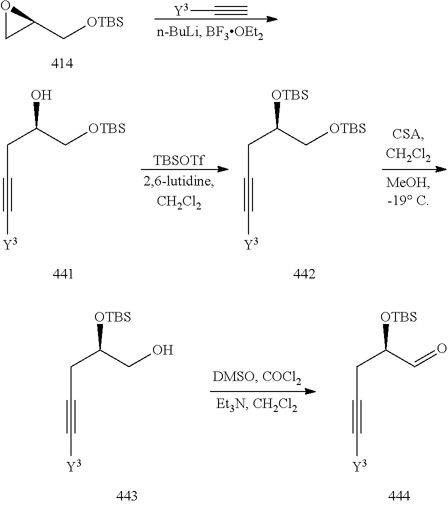 [0578] Y³=isopropyl, cyclohexyl, phenyl, 4fluorophenyl, 4-methoxyphenyl, 3,4-dichlorophenyl (440)

Synthesis of Compound 441. The epoxide opening of compound 414 was performed according to the procedure described for the production of Compound 415 in Example 4 using the appropriate alkyne. Purification by flash chromatography (silica, 95:5 hexanes/ethyl acetate when Y³=phenyl and cyclohexyl; 9:1 hexanes/ethyl acetate when Y³=phenyl, 4-fluorophenyl, 4-methoxyphenyl or 3,4,-dichlorophenyl) afforded 441.

Synthesis of Compound 442. The protection of compound 441 was performed according to the procedure described for the production of Compound 416 in Example 4. Purification by flash chromatography (silica, 95:5 hexanes/ethyl acetate when Y³=isopropy and cyclohexyl; 98.2 hexanes/ethyl acetate when Y³=phenyl; 4:1 hexanes/ethyl acetate when Y³=4-fluorophenyl, 4-methoyphenyl or 3,4,-dichlorophenyl) afforded 442.

Synthesis of Compound 443. The deprotection of compound 442 was performed according to the procedure described for the production of Compound 417 in Example 4. Purification by flash chromatography (silica, 95:5 hexanes/ethyl acetate when Y³=isopropy and cyclohexyl; 3:7 hexanes/ethyl acetate when Y³=phenyl, 4-fluorophenyl, 4-methoxyphenyl or 3,4,-dichlorophenyl) afforded 443.

Synthesis of Compound 444. The oxidation of compound 443 was performed according to the procedure described for the production of Compound 418 in Example 4. Purification by flash chromatography (silica, 95:5 hexanes/ethyl acetate when Y³=isopropy and cyclohexyl; 9:1 hexanes/ethyl acetate when Y³=phenyl, 4-fluorophenyl, 4-methoxyphenyl or 3,4,-dichlorophenyl) afforded 444.

### Example 10

### Syntheses of Compounds 306, 307, 308, 309, 310, 311, 314, 336, 337, 338, 339, 340, 341 and 342

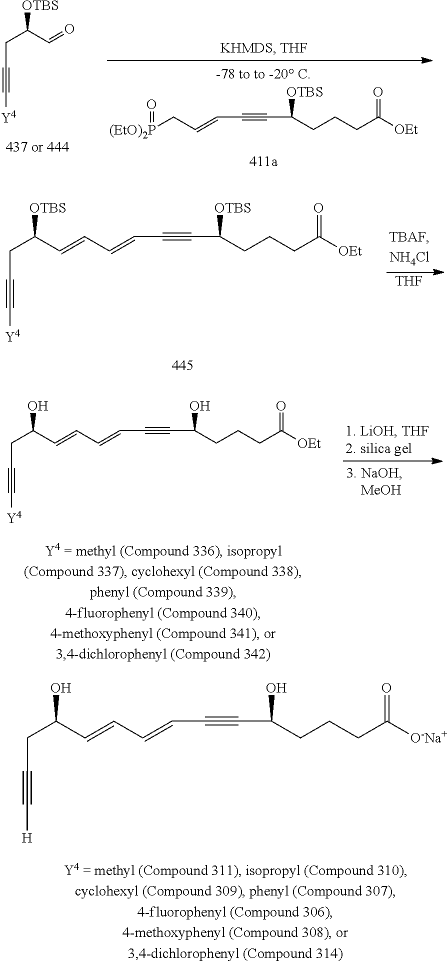

Synthesis of Compound 445. The coupling of compound 437 or 444 with compound 411a was performed according to procedure used to produce compound 433 in Example 6. Purification by flash chromatography (silica, hexanes to 95:5 hexanes/ethyl acetate) afforded 445.

Synthesis of Compounds 336, 337, 338, 339, 340, 341 and 342. The desilylation of compound 445 was performed according to the method used to produce compounds 326, 327 and 328 in Example 6. Purification by flash chromatography (silica, hexanes to 7:3 hexanes/ethyl acetate when Y⁴=isopropyl or cyclohexyl; 3:2 hexanes/ethyl acetate when Y⁴=phenyl, 4-fluorophenyl, 4-methoxyphenyl, or 3,4-dichlorophenyl) afforded the desired compound.

Compound 337: 45% yield: ¹H NMR (500 MHz, MeOD) δ 6.56 (dd, J=15.5, 10.5 Hz, 1H), 6.33 (dd, J=15.2, 10.7 Hz, 1H), 5.87 (dd, J=15.2, 6.2 Hz, 1H), 5.67 (dd, J=15.5, 1.0 Hz, 1H), 4.44 (td, J=6.2, 1.7 Hz, 1H), 4.18 (q, J=6.5 Hz, 1H), 4.12 (q, J=7.0 Hz, 2H), 2.53-2.46 (m, 1H), 2.39 (AB ddd, j=16.5, 5.5 2.5 Hz, 1H), 2.36 (t, J=7.5 Hz, 2H), 2.29 (AB ddd, J=16.3, 7.2, 2.2 Hz, 1H), 1.81-167 (m, 4H), 1.24 (t, J=7.2 Hz, 3H), 1.11 (d, J=6.5 Hz, 6H).

Compound 338: 89% yield: ¹N NMR (500 MHz, MeOD) δ 6.56 (dd, J=15.2, 10.7 Hz, 1H), 6.33 (dd, J=16.0, 11.0 Hz, 1H), 5.87 (dd, J=15.2, 6.2 Hz, 1H), 5.67 (dd, J=15.5, 1.0 Hz, 1H), 4.44 (td, J=6.2, 1.5 Hz, 1H), 4.19 (q, J=6.3 Hz, 1H), 4.12 (q, J=7.2 Hz, 2H), 2.42 (AB ddd, J=16.0, 5.5, 2.0 Hz, 1H), 2.36 (t, J=7.2 Hz, 2H), 2.35-2.28 (m, 2H), 1.79-1.66 (m, 8H), 1.49-1.28 (m, 6H), 1.24 (t, J=7.2 Hz, 3H); ESI MS m/z 395 [M+Na^{+]}⁺.

Compound 339: 71% yield: ¹H NMR (500 MHz, CDCl₃) δ 7.45-7.36 (m, 2H) 7.35-7.28 (m, 3H), 6.60 (dd, J=15.6, 10.9 Hz, 1H), 5.92 (dd, J=15.3, 5.9 Hz, 1H), 5.67 (dd, J=15.4, 1.3 Hz, 1H), 4.54 (qd, J=7.2, 1.6 Hz, 1H), 4.44 (pentet, J=5.5 Hz, 1H), 4.13 (q, J=7.2 Hz, 2H), 2.73 (dd, J=16.7, 5.5 Hz, 1H), 2.67 (dd, J=16.7, 6.5 Hz, 1H), 2.37 (t, J=6.7 Hz, 2H), 2.13 (d, J=4.9 Hz, 1H), 1.95 (d, J=5.5 Hz, 1H), 1.88-170 (m, 4H), 1.26 (t, J=7.2 Hz, 3H); ¹³C NMR (125 MHz, CDCl₃) δ 173.5, 141.2, 136.6, 131.7, 130.1, 128.3, 128.1, 123.1, 111.3, 92.6, 85.1, 84.1, 83.5, 70.3, 62.6, 60.4, 37.1, 33.8, 28.7, 20.6, 14.2.

Compound 340: 56% yield: ¹H NMR (500 MHz, CDCl₃) δ 7.37 (dd, J=8.8, 5.4 Hz, 2H), 6.99 (t, J=8.7 Hz, 2H), 6.60 (dd, J=15.5, 10.9 Hz, 1H), 6.38 (dd, J=15.2, 10.8 Hz, 1H), 5.91 (dd, J=15.3, 5.9 Hz, 1H), 5.66 (dd, J=15.5, 1.2 Hz, 1H), 4.54 (br q, J=5.5 Hz, 1H), 4.44 (pentet, J=5.3 Hz, 1H), 4.14 (q, J=7.2 Hz, 2H), 2.71 (dd, J=16.7, 5.6 Hz, 1H), 2.66 (dd, J=16.7, 6.5 Hz, 1H), 2.37 (t, J=7.0 Hz, 2H), 2.08 (d, J=4.9 Hz, 1H) 1.94 (d, J=5.4 Hz, 1H), 1.87-1.69 (m, 4H) 1.26 (t, J=7.1 Hz, 3H; ¹³C NMR (125 MHz, CDCl₃) δ 173.5, 161.4, 141.1, 136.5, 133.6, 133.5, 130.2, 119.2, 115.5, 115.4, 111.4, 92.6, 84.8, 84.0, 82.4, 70.3, 62.6, 60.4, 37.1, 33.8, 28.5, 20.6, 14.2.

Compound 341: 78% yield: ¹H NMR (500 MHz, CDCl₃) δ 7.33 (d, J=8.8 Hz, 2H) 6.82 (d, J=8.8 Hz, 2H), 6.60 (dd, J=15.5, 10.9 Hz, 1H), 6.37 (dd, J=15.2, 10.8 Hz, 1H), 5.91 (dd, J=15.3, 5.9 Hz, 1H), 5.65 (dd, J=15.5, 1.2 Hz, 1H), 4.53 (br s, 1H), 4.42 (br s, 1H), 4.14 (q, J=7.1 Hz, 2H), 3.81 (s, 3H), 2.71 (dd, J=16.6, 5.4 Hz, 1H), 2.65 (dd, J=16.6, 6.6 Hz, 1H), 2.37 (t, J=6.7 Hz, 2H), 2.13 (d, J=4.4 Hz, 1H), 1.92 (d, J=4.7 Hz, 1H), 1.87-1.71 (m, 4H), 12.6 (t, J=7.2 Hz, 3H); ¹³C NMR (125 MHz, CDCl₃) δ 173.5, 159.4, 141.2, 136.7, 133.1, 130.0, 122.4, 121.4, 120.0 119.9, 115.2, 113.9, 111.2, 92.5, 84.1, 83.4, 83.3, 70.3, 62,6, 60.4, 55.3, 37.1, 33.8, 28.7, 20.6, 14.2.

Compound 342: 89% yield: ¹H NMR (500 MHz, CDCl₃) δ 7.48 (dd, J=1.9 Hz, 1H), 7.36 (d, J=8.3 Hz, 1H), 7.21 (dd, J=8.3, 1.9 Hz, 1H), 6.59 (dd, J=15.5, 10.9 Hz, 1H), 6.37 (dd, J=15.3, 10.9 Hz, 1H), 5.89 (dd, J=15.2, 6.0 Hz, 1H), 5.67 (dd, J=15.6, 1.4 Hz, 1H), 4.58-4.49 (m, 1H) (pentet, J=5.3 Hz, 1H), 4.13 (q, J=7.1 Hz, 2H), 2.72 (dd, J=16.8, 5.6 Hz, 1H), 2.67 (dd, J=16.8, 6.4 Hz, 1H), 2.37 (t, J=6.7 Hz, 2H), 2.01 (d, J=4.8 Hz, 1H) 1.92 (d, J=5.5 Hz, 1H), 1.87-1.70 (m, 4H) 1.26 (t, J=7.3 Hz, 3H; ¹³C NMR (125 MHz, CDCl₃) δ 173.5, 141.0, 136.3, 133.4, 132.5, 130.9, 130.3, 123.2, 115.6, 92.8, 84.0, 70.2, 62.6, 60.4, 37.1, 33.8, 28.5, 20.6, 14.3.

Synthesis of Compound 336. the desilylation of compound 445 was performed according to the method used to produce compounds 326, 327 and 328 in Example 6. Purification by flash chromatography (silica, 7:33 to 3:2 hexanes/ethyl acetate) afforded an intermediate that was isomerized by dissolving in methylene chloride (50 mL), adding iodine crystals (0.50 g, 0.197 mmol) and stirring room temperature for 15 min in a dark hood. Then at 10% (wt/v) solution of aqueous sodium thiosulfate (50 mL) was added. The organic layer was separated and washed with water (2×100 mL), dried over sodium sulfate, filtered, and concentrated. Purification by flash chromatography (silica, 5:45:50 to 20:30:50 methyl tert-butyl ether/hexanes/dichloromethane) and careful peak splitting afforded Compound 336 in 38% yield: ¹H NMR (500 MHz, MeOD) δ 6.56 (dd, J=15.5, 11.0 Hz, 1H), 6.33 (dd, J=15.0, 11.0 Hz, 1H), 5.88 (dd, J=15.2, 5.8 Hz, 1H), 5.68 (d, J=15.5 Hz, 1H), 4.45-4.42 (m, 1H) 4.19 (q, J=6.0 Hz, 1H), 4.12 (q, J=7.0 Hz, 2H), 2.36 (t, J=7.0 Hz, 2H), 2.36-2.26 (m, 2H) 1.79-1.64 (m, 4H) 1.74 (t, J=2.5 Hz, 3H), 1.24 (t, J=7.2 Hz, 3H).

Synthesis of Compounds 306, 307, 308, 309, 310, and 314. The hydrolysis of the foregoing ethyl esters was performed according to the procedure for producing compounds 303, 304 and 305 in Example 6.

Compound 306: 95% yield: ¹H NMR (500 MHz, CD₃OD) δ 7.29 (dd, J=8.8, 5.4 Hz, 2H), 6.93 (t, J=8.8 Hz, 2H), 6.50 (dd, J=15.5, 10.8 Hz, 1H), 6.29 (dd, J=15.2, 10.8 Hz, 1H), 5.83 (dd, J=15.2, 6.2 Hz, 1H), 5.59 (dd, J=15.2, 6.2 Hz, 1H), 4.35 (m, 1H) 4.23 (q, J=6.1 Hz, 1H), 2.54 (dd, J=16.6, 6.1 Hz, 1H), 2.49 (dd, J=16.7, 7.66 Hz, 1H), 2.10 (br t, J=6.9, 2H) 172-1.49 (m, 4H):APCl MS m/z 355 [M−H]:HPLC (Method 1), 98.0% (AUC).

Compound 307: 86% yield: ¹H NMR (500 MHz, CD₃OD) δ 7.41-7.32 (m, 2H) 7.32-7.22 (m, 3H) 6.60 (dd, J=15.5, 10.8 Hz, 1H), 6.39 (dd, J=15.3, 10.9 Hz 1H), 5.94 (dd, J=15.2, 6.2 Hz, 1H), 5.70 (d, J=15.5 Hz, 1H), 4.78-4.40 (m, 1H) 4.33 (q, J=6.1 Hz, 1H), 2.65 (dd, J=16.6, 6.0 Hz, 1H), 2.59 (dd, J=16.6, 6.6 Hz, 1H), 2.19 (t, J=7.1 Hz, 2H), 1.81-1.63 (m, 4H) ; APCl MS m/z 337 [M−H]:HPLC 97.9% (AUC).

Compound 308: 95% yield: ¹H NMR (500 MHz, CD₃OD) δ 7.19 (d, J=8.8 Hz, 2H), 6.74 (d, J=8.8 Hz 2H), 6.50 (dd, J=15.5, 10.8 Hz, 1H), 6.29 (dd, J=15.4, 11.0 Hz, 1H), 5.84 (dd, J=15.2, 6.2 Hz, 1H), 5.59 (dd, J=15.6, 1.1 Hz, 1H), 4.34 (m, 1H) 4.22 (q, J=6.1 Hz, 1H), 3.68 (s, 3H) 2.53 (dd, J=16.6, 6.0 Hz, 1H), 2.47 (dd, J=16.6, 6.7 Hz 1H), 2.10 (t, J=6.9 Hz, 2H), 1.72-1.52 (m, 4H): APCl MS m/z 367 [−H]:HPLC (Method 1) 95.6% (AUC).

Compound 309: 97% yield: ¹H NMR (500 MHz, MeOD) δ 6.56 (dd, J=15.5, 11.0 Hz, 1H), 6.33 (dd, J=15.2, 10.7 Hz 1H), 5.87 (dd, J=15.2, 6.2 Hz, 1H), 5.67 (d, J=15.5 Hz, 1H), 4.46-4.44 (m, 1H), 4.19 (q, J=6.3 Hz, 1H), 2.42 (AB ddd, J=16.0, 5.5, 2.0 Hz, 1H), 2.37-2.28 (m, 2H) 2.19 (t, J=7.0 Hz, 2H), 1.77-1.66 (m, 8H) 1.52-2.0 (m, 6H); ESI MS m/z 343 [M−H]:HPLC (Method 1) >97.2% (AUC).

Compound 310: 53% yield: ¹H NMR (500 MHz, MeOD) δ 6.56 (dd, J=15.5, 11.0 Hz, 1H), 6.32 (dd, J=15.2, 10.7 Hz 1H), 5.86 (dd, J=15.2, 6.2 Hz, 1H), 5.67 (dd, J=14.5 Hz, 1H), 4.45-4.44 (m, 1H), 4.18 (q, J=6.4 Hz, 1H), 2.55-2.46 (m, 1H), (AB ddd, J=16.4, 6.0. 2.0 Hz, 1H), 2.30 (AB ddd, J=16.3, 7.2, 2.0 Hz, 1H) 2.20 (t, J=6.5 Hz, 2H), 1.81-1.67 (m, 4H), 1.11 (d, J=7.0 Hz, 6H); ESI MS m/z 303 [M−H]:HPLC (Method 1) 95.7% (AUC).

Compound 311: 89% yield: ¹H NMR (500 MHz, MeOD) δ 6.56 (dd, J=15.5, 10.5 Hz, 1H), 6.32 (dd, J=15.2, 11.2 Hz 1H), 5.88 (dd, J=15.5, 6.0 Hz, 1H), 5.67 (d, J=15.5 Hz, 1H), 4.45-4.44 (m, 1H), 4.18 (q, J=6.2 Hz, 1H), 2.39-2.27 (m, 2H) 2.29 (t, J=7.2 Hz, 2H), 1.77-1.171 (m, 4H), 1.74 (t, J=2.5 Hz, 3H); ESI MS m/z 299 [M+H]⁺:HPLC (Method 1) 96.6% (AUC).

Compound 314: 95% yield: ¹H NMR (500 MHz, CD₃OD) δ 7.43 (d, J=1.9 Hz, 1H), 7.35 (d, J=8.3 Hz, 1H), 7.19 (dd, J=8.3, 1.9 Hz, 1H), 6.50 (dd, J=15.5, 10.8 Hz, 1H), 6.29 (dd, J=15.3, 10.9 Hz, 1H), 5.82 (dd, J=15.2, 6.2 Hz, 1H), 5.61 (dd, J=15.6. 1.1 Hz, 1H), 4.38-4.31 (m, 1H), 4.25 (q, J=6.2 Hz, 1H), 2.56 (dd, J=16.8, 6.2 Hz, 1H), 2.52 (dd, J=16.8, 6.3 Hz, 1H) 2.10 (t, J=7.0 Hz, 2H) 1.71-1.53 (m, 4H); APCl MS m/z 405 [M−H]:HPLC (Method 1) 98.5% (AUC).

### Example 11

### Synthesis of Compounds 201 and 343

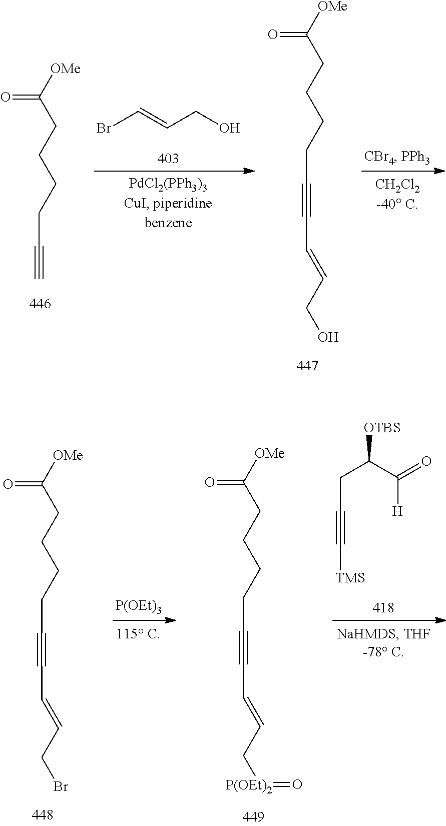

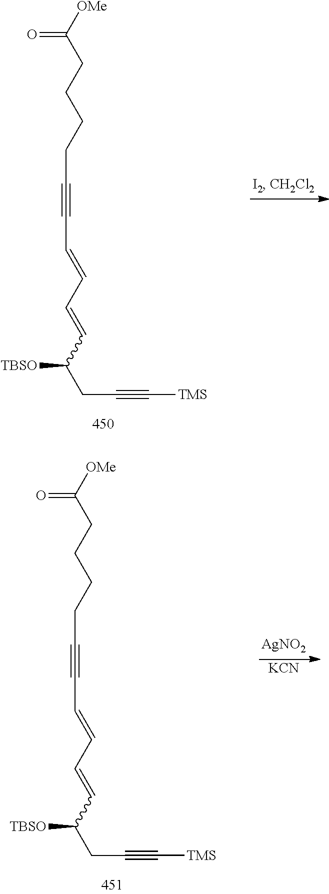 [SMILES: COC(=O)CCCCC#C/C=C/C=C[C@@H](CC#C[Si](C)(C)C)O[Si](C)(C)C(C)(C)C ; COC(=O)CCCCC#C/C=C/C=C[C@@H](CC#C[Si](C)(C)C)O[Si](C)(C)C(C)(C)C ; "AgNO2" ; "KCN"]

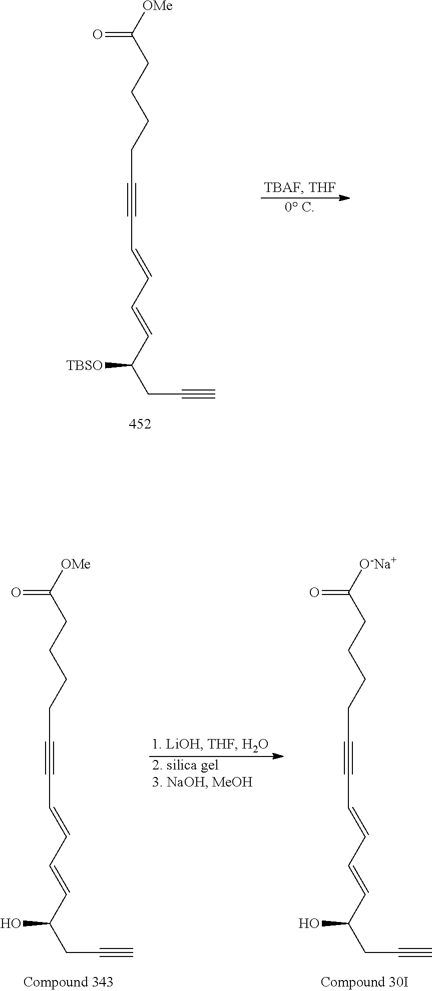

Synthesis of Compound 447. A solution of the acetylene 446 was added to a stirred mixture of the bromo allylic alcohol 403 (1.3× molar excess), bisdiphenylphosphino palladium(II) chloride (0.036 g, 0.05 mmol) and copper(I) iodide (10% molar amount) in benzene (25 mL) under an inert atmosphere of argon. Piperidine (5× molar excess) was then added and the reaction mixture stirred at room temperature and monitored by the until complete. The reaction was then diluted with diethyl ether (100 mL) and water (25 mL). The organic layer was separated and washed with brine (50 mL), dried over sodium sulfate, filtered and concentrated. Purification by flash chromatography (silica, hexanes to 3:1 hexanes/ethyl acetate) afforded 447 in 83% yield.

Synthesis of Compound 448. The bromination of compound 447 was performed according to the method used to produce compound 410 in Example 2. Purification by flash chromatography (silica, hexanes to 3:1 hexanes/ethyl acetate) afforded 448 in 89% yield.

Synthesis of Compound 449. The formation of phosphonate 449 from compound 448 as performed according to the method used to produce compound 441 in Example 2. Purification by flash chromatography (silica, hexanes to 3:7 hexanes/ethyl acetate) afforded 449 in 81% yield.

Synthesis of Compound 450. The coupling of compounds 450 and 418 was performed according to the method used to produce compound 433 in Example 6. Purification by flash chromatography (silica, hexanes to 19:1 hexanes/ethyl acetate) afforded 450 in 29% yield.

Synthesis of Compound 451. The isomerisation of compound 450 was performed according to the method used in the production of Compound 336 in Example 10 and afforded 451 in quantitative yield.

Synthesis of Compound 452. To a stirred solution of compound 451 in terahydrofuran (12.9 mL) and absolute ethanol (6.5 mL) at 0° C. under nitrogen was added dropwise a solution of silver(I) nitrate (0.689 g, 4.06 mmol) in tetrahydrofuran (6.5 mL) and water (6.5 mL) and a yellow precipitate was formed. The ice-bath was replaced with a room temperature water bath and the reaction mixture was stirred for 1.5 h. The reaction mixture was then cooled to 0° C. and a solution of potassium cyanide (0.451 g, 6.92 mmol) in water (6.5 mL) was added dropwise. The ice-bath was removed and the reaction was stirred for 15 min and then filtered through diatomaceous earth. The filter cake was washed with diethyl ether (50 mL), water (50 mL) then with ethyl acetate (50 mL) and finally with water (50 mL). the aqueous layer of the filtrate was separated and extracted with ethyl acetate (50 mL). The combined organic layers were washed with brine (50 mL), dried over magnesium sulfate, filtered and concentrated. Purification by flash chromatography (silica, hexanes to 85:15 hexanes/ethyl acetate) afforded 452 (0.272 g, 52%) as a colorless oil: ¹H NMR (500 MHz, CDCl₃) δ 6.50 (dd, J=15.5, 10.8 Hz, 1H), 6.24 (dd, J=15.1, 10.8 Hz 1H), 5.80 (dd, J=15.1, 10.8 Hz, 1H), 5.58 (d, J=15.5 Hz, 1H), 4.31 (q, J=6.3 Hz, 1H), 3.67 (s, 3H) 2.38-2.67 (m, 5H), 1.98 (t, J=2.6 Hz, 1H), 1.80-1.68 (m, 2H), 162-1.52 (m, 2H), (s, 9H), 0.08 (s, 3H), 0.05 (s, 3H): ¹³C NMR (125 MHz, CDCl₃) δ 173.9, 139.9, 136.6, 129.5, 112.0, 92.5, 81.0, 80.1, 71.5, 70.1, 51.5, 33.6, 28.5, 28.1, 25.8, 24.1, 19.3 18.2, −4.6, −4.8.

Synthesis of Compound 343. The desilylation of compound 452 was performed according to the method used to produce Compounds 326, 327 and 328 in Example 6. Purification by flash chromatography (silica, hexanes to 85:15 hexanes/ethyl acetate) afforded Compound 343 in 66% yield. ¹H NMR (500 MHz, CDCl₃) δ 6.50 (dd, J=15.5, 10.8 Hz, 1H), 6.32 (dd, J=15.3, 10.8 Hz 1H), 5.80 (dd, J=15.2, 6.1 Hz, 1H), 5.62 (d, J=15.5 Hz, 1H), 4.35 (dq, J=5.4, 5.4 Hz, 1H), 3.67 (s, 3H) 2.48 (ABdd, J_{AB}=16.6 Hz, J=5.4, 2.6 Hz, 2H) 2.40-2.29 (m, 4H) 2.07 (t, J=2.6 Hz 1H), 2.01 (d, J=4.8 Hz, 1H), 1.81-1.68 (m, 2H), 163-1.49 m, 2H): ¹³C NMR (125 MHz, CDCl₃) δ 173.9, 139.5, 134.9, 134.7, 112.9, 93.0, 80.0, 79.9, 71.1, 70.1, 51.5, 33.6, 28.1, 27.6, 24.1, 19.3.

Synthesis of Compound 301. The hydrolysis of compound 343 was performed according to the method used to produce compounds 326, 327 and 328 in Example 6. Purification by RP preparative chromatography (3:7 acetonitrile/water) afforded 35 in 17% yield: ¹H NMR (500 MHz, CDCl₃) δ 6.45 (dd, J=15.4 Hz, 10.8 Hz, 1H), 6.30 (dd, J=15.1, 11.2 Hz 1H), 5.82 (dd, J=15.1, 6.2 Hz, 1H), 5.61 (d, J=15.4 Hz, 1H), 4.22 (q, J=6.3 Hz, 1H), 2.38 (obscured ABdd, J_{AB}=16.5 Hz, J=6.0, 2.6 Hz, 2H), 2.33 (td, J=7.1, 1.8 Hz, 2H), 2.27 (td, J=2.7, 0.7 Hz, 1H) 2.17 (t, J=7.5 Hz, 2H), 1.76-1.64 (m, 2H), 1.59-1.48 (m, 2H); ESI MS m/z 245 [M−H]:HPLC (Method 1) >99% (AUC).

### Example 12

### Biological Activity as Assessed Through Animal Models of Disease

The biological activity, such as anti-inflammatory activity, of one or more of a compound of formula A, a compound of any one of Formulae 1-49 or I-III, a lipoxin compound, an oxylipin compound, or a combination of aspirin and an omega-3 fatty acid alone, or in combination with one or more of a PPAR agonist (e.g., a PPARα, PPARβ/δ, or a PPARγ agonist), an LXR agonist (e.g., an LXRα or LXRβ agonist), an RXR agonist (e.g., an RXRα, RXRβ, or an RXRγ agonist), an HNF-4 agonist, or a sirtuin-activating compound can be assessed using techniques and animal models of diseases well known in the art, such as those discussed below.

### Assay for Anti-Inflammatory Effect

Human leukocytes (e.g., monocytes, lymphocytes, and neutrophils) are subjected in vitro to one or more proinflammatory and/or proliferative stimuli and secreted mediators of inflammation, such as cytokines, chemokines, and/or components involved in intracellular kinase pathways involved in their formation, are measured. Differences in these measurements between control cells and cells preincubated with a test anti-inflammatory composition, such as a composition comprising a compound of formula A, a compound of any one of formulae 1-49 or I-III, a lipoxin compound, an oxylipin compound, or a combination of aspirin and an omega-3 fatty acid and a PPAR agonist (e.g., a PPARα, PPARβ/δ, or a PPARγ agonist), an LXR agonist (e.g., an LXRα or LXRβ agonist), an RXR agonist (e.g., an RXRα, RXRβ, or an RXRγ agonist), an HNF-4 agonist, or a sirtuin-activating compound, in inhibiting the formation of these mediators can be determined over different time courses and/or using a wide range of concentrations of the test composition.

### INCORPORATION BY REFERENCE

All publication and patents mentioned herein are hereby incorporated by reference in their entirety as if each individual publication or patent were specially and individually indicated to be incorporated by reference. In particular, compounds of formula A or formulae 1-49 disclosed in US 2003/0191184, WO 2004/014835, WO 2004/078143, U.S. Pat. No. 6,670,396, US 2003/0236423, and US 2005/0228047, lipoxin compounds disclosed in US 2002/0107289, US 2004/0019110, US 2006/0009521, US 2005/0203148, and US 2005/011443, oxylipin compounds disclosed in WO2005/055965, WO 2007/090162, and WO2008/103753, derivatives and/or analogs of eicosapentaenoic acid or docosahexaenoic acid disclosed in WO 2005/089744, US 2004/0044050, US 2004/0116408 and US 2005/026155 and aspirin-triggered lipid mediators disclosed in U.S. 7,053,230 are incorporated by reference as suitable for use in compositions and methods of the present invention. In case of conflict of structures or naming of compounds between the present application and the referenced patent publications listed above, the present application, including any definitions herein, will control.

### EQUIVALENTS

While specific embodiments of the subject invention have been discussed, the above specification is illustrative and not restrictive. Many variations of the invention will became apparent to those skilled in the art upon review of this specification and the claims below. The full scope of the invention should be determined by reference to the claims, along with their full scope of equivalents, and the specification, along with such variations.

1. A method of treating inflammatory disease in a patient comprising conjointly administering to said patient in need thereof a first agent selected from a PPAR agonist, an LXR agonist, an RXR agonist, an HNF-4 agonist, or a sirtuin-activating compound and a second agent selected from a compound of formula A, a compound of any one of formulae 1-49 or I-III, a lipoxin compound, an oxylipin compound, a prodrug of any of the foregoing, or a pharmaceutically acceptable salt of any of the foregoing.

2. The method according to claim 1, further comprising administering to the patient in need thereof an anti-inflammatory agent other than the first agent or the second agent.

3. The method according to claim 1, wherein the amount of the first agent administered to the patient is less than an amount that achieves a therapeutic effect when administered in the absence of the second agent.

4. The method according to claim 1, wherein the second agent is administered to the patient in an amount less than an amount that achieves an anti-inflammatory effect in the absence of the first agent.

5. A method of reducing the dose of a first agent selected from a PPAR, LXR, RXR, or HNF-4 agonist, or a sirtuin-activating compound required to produce an anti-inflammatory effect in a patient in need thereof, comprising conjointly administering to the patient a second agent selected from a compound of formula A, a compound of any one of formulae 1-49 or I-III, a lipoxin compound, an oxylipin compound, a prodrug of any of the foregoing, or a pharmaceutically acceptable salt of any of the foregoing with the first agent.

6-9. (canceled)

10. The method according to claim 1, wherein the second agent is selected from a compound of any one of Formulae 1 to 132.

11. A composition comprising:
a first agent selected from a PPAR agonist, a LXR agonist, an RXR agonist, a HNF-4 agonist, or a sirtuin-activating compound;
a second agent selected from a compound of formula A, a compound of any one of formulae 1-49 or I-III, a lipoxin compound, an oxylipin compound, a prodrug of any of the foregoing, or a pharmaceutically acceptable salt of any of the foregoing; and
a pharmaceutically acceptable carrier.

12. The composition according to claim 11, further comprising an anti-inflammatory agent other than the first agent or the second agent.

13. The composition according to claim 11, wherein the second agent is selected from a compound of any one of Formulae 1 to 132.

14. The method according to claim 1, wherein the PPAR agonist is selected from GW409544, LY-518674, LY-510929, TZD18, LTB4, oleylethanolamide, LY-465608, pirinixic acid, fatty acids, ragaglitazar, AD-5061, fenofibric acid, GW7647, GW9578, TAK-559, KRP-297/MK-0767, eicosatetraenoic acid, farglitazar, reglitazar, DRF 2519, pristanic acid, bezafibrate, clofibrate, 8S-hydroxyeicosatetraenoic acid, GW2331, NS-220, pterostilbene, tetradecylglycidic acid, ortylthiopropionic acid, WY14643, ciprofibrate, gemfibrozil, muraglitazar, tesaglitazar, eicosanoids, GW0742X, GW2433, GW9578, GW0742, L-783483, GW501516, retinoic acid, L-796449, L-165461, L-165041, SB-219994, LY-510929, AD-5061, L-764406, GW0072, nTzDpa, troglitazone, LY-465608, pioglitazone, SB-219993, 5-aminosalicyclic acid, GW1929, L-796449, GW7845, 2-cyano-3,12-dioxooleana-1,9-dien-28-oic acid, L-783483, L-165461, AD5075, fluorenylmethoxycarbonyl-L-leucine, CS-045, indomethacin, rosiglitazone, SB-236636, GW2331, PAT5A, MCC555, linoleic acid, bisphenol A diglycidyl ether, GW409544, GW9578, TAK-559, reglitazar, GW9578, ciglitazone, DRF2519, LG10074, ibuprofen, diclofenac, fenofibrate, naviglitazar, or pharmaceutically acceptable salts thereof.

15. The composition according to claim 11, wherein the PPAR agonist is selected from GW409544, LY-518674, LY-510929, TZD18, LTB4, oleylethanolamide, LY-465608, pirinixic acid, fatty acids, ragaglitazar, AD-5061, fenofibric acid, GW7647, GW9578, TAK-559, KRP-297/MK-0767, eicosatetraenoic acid, farglitazar, reglitazar, DRF 2519, pristanic acid, bezafibrate, clofibrate, 8S-hydroxyeicosatetraenoic acid, GW2331, NS-220, pterostilbene, tetradecylglycidic acid, ortylthiopropionic acid, WY14643, ciprofibrate, gemfibrozil, muraglitazar, tesaglitazar, eicosanoids, GW0742X, GW2433, GW9578, GW0742, L-783483, GW501516, retinoic acid, L-796449, L-165461, L-165041, SB-219994, LY-510929, AD-5061, L-764406, GW0072, nTzDpa, troglitazone, LY-465608, pioglitazone, SB-219993, 5-aminosalicyclic acid, GW1929, L-796449, GW7845, 2-cyano-3,12-dioxooleana-1,9-dien-28-oic acid, L-783483, L-165461, AD5075, fluorenylmethoxycarbonyl-L-leucine, CS-045, indomethacin, rosiglitazone, SB-236636, GW2331, PAT5A, MCC555, linoleic acid, bisphenol A diglycidyl ether, GW409544, GW9578, TAK-559, reglitazar, GW9578, ciglitazone, DRF2519, LG10074, ibuprofen, diclofenac, fenofibrate, naviglitazar, or pharmaceutically acceptable salts thereof.

16. The method according to claim 1, wherein the LXR agonist is selected from TO901317, GW3965, T1317, acetyl-podocarpic dimer (APD), or pharmaceutically acceptable salts thereof.

17. The composition according to claim 11, wherein the LXR agonist is selected from TO901317, GW3965, T1317, acetyl-podocarpic dimer (APD), or pharmaceutically acceptable salts thereof.

18. The method according to claim 1, wherein the RXR agonist is selected from LG 100268, LGD 1069, AGN 194204, 9-cis-retinoic acid, AGN 191701, bexarotene, BMS 649, or pharmaceutically acceptable salts thereof.

19. The composition according to claim 11, wherein the RXR agonist is selected from LG 100268, LGD 1069, AGN 194204, 9-cis-retinoic acid, AGN 191701, bexarotene, BMS 649, or pharmaceutically acceptable salts thereof.

20-43. (canceled)
